(19) United States
(12) Patent Application Publication
(10) Pub. No.: US 2020/0171132 A1
(43) Pub. Date: Jun. 4, 2020

(54) METHODS OF INHIBITING FUNGAL CERAMIDE SYNTHASE FOR TREATMENT OF CRYPTOCOCCUS NEOFORMANS INFECTION

(71) Applicant: The Research Foundation for the State University of New York, Albany, NY (US)

(72) Inventors: Maurizio Del Poeta, Mount Sinai, NY (US); Mansa Munshi, Boston, MA (US); Krupanandan Haranahalli, East Setauket, NY (US); Karen You, Hawthorne, NY (US); Iwao Ojima, Port Jefferson, NY (US)

(73) Assignee: The Research Foundation for the State University of New York, Albany, NY (US)

(21) Appl. No.: 16/495,605

(22) PCT Filed: Mar. 20, 2018
(86) PCT No.: PCT/US2018/023413

§ 371 Date: Sep. 19, 2019

Related U.S. Application Data
(60) Provisional application No. 62/620,080, filed on Jan. 22, 2018.
(60) Provisional application No. 62/473,742, filed on Mar. 20, 2017.

(51) Int. Cl. A61K 38/46; A61K 31/4155; A61K 31/4985; A61P 31/10; A61K 31/506; A61K 31/351; A61K 31/47; A61K 31/4196; A61K 31/395; A61K 31/55; A61K 31/5377; A61K 31/519; C12N 9/22; A61K 31/553; A61K 31/439; A61K 31/7105; C12N 15/113; C12Q 1/48; A61K 31/4045

(52) U.S. Cl. CPC A61K 38/465; C12N 2800/80; A61K 31/4985; A61P 31/10; A61K 31/506; A61K 31/351; A61K 31/47; A61K 31/4196; A61K 31/5545; A61K 31/55; A61K 31/5377; A61K 31/519; C12N 9/22; A61K 31/553; A61K 31/439; A61K 31/7105; C12N 15/1137; C12Q 1/48; A61K 31/4045; C12N 2310/20; A61K 31/4155

(57) ABSTRACT

The present invention provides a method of inhibiting the growth of a fungus comprising contacting the fungus with an effective amount of an inhibitor so as to thereby inhibit the growth of the fungus, wherein the inhibitor inhibits ceramide synthase 1 (Cer1) in the fungal cells of the fungus. The present invention also provides method of treating a subject afflicted with a fungal infection comprising administering to the subject an effective amount of an inhibitor so as to treat the subject afflicted with the fungal infection, wherein the inhibitor inhibits ceramide synthase 1 (Cer1) in the fungal cells of the fungus.

This application claims priority of U.S. Provisional Application Nos. 62/620,080, filed Jan. 22, 2018 and 62/473,742, filed Mar. 20, 2017, the contents of each of which are hereby incorporated by reference.

### GOVERNMENT SUPPORT

This invention was made with government support under AI056168 awarded by the National Institutes of Health. The government has certain rights in the invention.

Throughout this application, certain publications are referenced in parentheses. Full citations for these publications may be found immediately preceding the claims. The disclosures of these publications in their entireties are hereby incorporated by reference into this application in order to describe more fully the state of the art to which this invention relates.

### REFERENCE TO SEQUENCE LISTING

This application incorporates-by-reference nucleotide sequences which are present in the file named “180320_90367-A-PCT_Sequence_LPT” which is 44 kilobytes in size, and which was created Mar. 19, 2018 in the IBM-PC machine format, having an operating system compatibility with MS-Windows, which is contained in the text file filed Mar. 19, 2018.

### BACKGROUND OF THE INVENTION

Cryptococcus neoformans (Cn) is a pathogenic fungus that presents a leading cause of fungal meningoencephalitis worldwide. Recent reports reveal an annual 278,000 cases of cryptococcal antigenaemia, with cryptococcal meningitis being the cause of 15% AIDS related deaths (Rajasingham et al., 2017). Naturally occurring cases of cryptococcosis begin by inhalation of fungal spores. Once in the lung, the outcome depends largely on the immune system of the individual. In a situation of suppressed immunity, infection may lead to pneumonia and cryptococcal meningitis. In cases of immunocompetence, these cells are either cleared or may establish a latent infection that will later disseminate upon future immunosuppression. Once Cn enters the lung, the cells are typically engulfed by an alveolar macrophage where they can survive and replicate. Similarly, Cn can survive and replicate well in extracellular spaces, such as alveoli, blood, and other tissues. Once engulfed, Cn can move between the phagolysosome and extracellular space without causing harm to the macrophage (Alvarez and Casadevall, 2006, Feldmesser et al., 2000).

The intracellular and extracellular environment in the host is distinguished by a prominent difference in pH. Within the phagolysosome, the environment pH is highly acidic, while the extracellular environment is typically neutral or slightly alkaline. Adaptation to these starkly contrasting environments is critical for Cn pathogenicity. There is little information regarding how Cn regulates its survival in these two host environments. Previous studies have shown sugar complexed sphingolipids to be essential for the survival of Cn when grown in media mimicking host acidic or alkaline conditions. Specifically, inositol or mannose containing sphingolipids are noted as important for the survival and replication of Cn in conditions similar to the phagolysosome (Luberto et al., 2001). Conversely, glucose containing sphingolipids have been indicated to be important for survival in conditions mimicking the extracellular environment (Rittershaus et al., 2006). Among sphingolipids, ceramides constitute the simplest class and the basic backbone that precedes other more complex sphingolipids (Aguilera-Romero et al., 2014). Acyl-CoA dependent ceramide synthases catalyze the formation of ceramide from a fatty acyl CoA and sphingoid base. Cryptococcal sphingolipids regulate signaling events that lead to the production of virulence factors (Singh and Del Poeta, 2011). Studies in C. albicans (Cheon et al., 2012), S. cerevisiae (Sc) (Kageyama-Yahara and Riezman, 2006), A. nidulans (Li et al., 2006) and P. pastoris (Ternes et al., 2011) show the presence of two distinct ceramide synthase enzymes. While a handful of studies reveal different characteristic functions of ceramide synthases in each eukaryotic species, there is still a lack of concrete evidence for the specific roles of ceramide synthases in the context of sphingolipid biosynthesis of fungi.

Currently, three classes of antifungal drugs (polyenes, azoles and echinocandins) are employed to treat cryptococcosis, aspergillosis or candidiasis. It is widely recognized that the introduction of antifungal (or generally, anti-infective) agents acting by a different but complimentary mode of action to existing therapeutics can provide a tremendous advantage over available treatment regimes, alone or in combination. Unfortunately, unlike cancer chemotherapy, there exist only a few treatment regimes that productively combine different antifungals to achieve better therapeutic outcomes and address drug resistance without the added burden of drug toxicity (Lewis).

### SUMMARY OF THE INVENTION

The present invention provides a method of inhibiting the growth of a fungus comprising contacting the fungus with an effective amount of an inhibitor so as to thereby inhibit the growth of the fungus,
wherein the inhibitor inhibits ceramide synthase 1 (Cer1) in the fungal cells of the fungus.

The present invention also provides a method of treating a subject afflicted with a fungal infection comprising administering to the subject an effective amount of an inhibitor so as to treat the subject afflicted with the fungal infection,
wherein the inhibitor inhibits ceramide synthase 1 (Cer1) in the fungal cells of the fungus.

### BRIEF DESCRIPTION OF THE FIGURES

FIG. 1A: Phylogenetic analysis of eukaryotic ceramide synthases. Cryptococcus neoformans has three ceramide synthases. The entire phylogenetic tree can be roughly divided into 3 major groups. Namely, human cerS genes, the second containing orthologs of CnCer1, and the third group consisting of Sc Lad and Lag1, AnlagA, CaLac1 and CnCer2 and CnCer3. ScLip1 is distinct from all of these genes.

FIG. 1B: Biochemical analysis of Cn ceramide synthases. Thin layer chromatography showing substrate specificity studies of Cn ceramide synthases. Ceramide synthase assays using NBD-sphingosine as a substrate. Lane 1 (left) is positive control using mammalian Cer1 microsomes. Right panel is negative control of each strain grown in 2% glucose.

FIG. 1C: Ceramide synthase assays using NBD-phytosphingosine as a substrate. Lane 1 (left) is positive control using mammalian Cer1 microsomes. Right panel is negative control of each strain grown in 2% glucose. Each horizontal panel represents a specific strain.

FIG. 2A: Survival studies of CBA/JCrHsd mice infected intranasally with WT, Δcer1, Δcer1+CER1, Δcer2 and Δcer3. n=10 mice per group. Data represented as Mean±SEM.

FIG. 2B: Histology of lung tissue for WT (at time of death) and for Δcer1 (day 60). Lung sections were stained with haematoxylin and eosin. (a and b) Lung of mice infected with WT (c and d) Lung of mice infected with Δcer1. Bar (a and c)=1000 μm (b and d)=20 μm.

FIG. 2C: Lung tissue burden analysis of WT, Δcer1 and Δcer1+CER1. Data represented as Mean±SEM.

FIG. 2D: Brain fungal burden analysis of WT, Δcer1 and Δcer1+CER1. n=3 mice at each time point. Data represented as Mean±SEM.

FIG. 3A: Sphingolipid biosynthetic pathway and ceramide species abundance of Cn ceramide synthase mutants and WT in distinct host conditions in vitro using MS analysis. Changes in specific lipid classes at pH 4.0. Data represented as mean±SEM.

FIG. 3B: Sphingolipid biosynthetic pathway and ceramide species abundance of Cn ceramide synthase mutants and WT in distinct host conditions in vitro using MS analysis. Changes in specific lipid classes at pH 7.4. Data represented as mean±SEM.

FIG. 3C: Abundance of complex sphingolipid species in WT and ceramide synthase mutants at pH 4.0. Data represented as mean±SEM.

FIG. 3D: Abundance of complex sphingolipid species in WT and ceramide synthase mutants at pH 7.4. Data represented as mean±SEM.

FIG. 4A: In vitro growth of WT, Δcer1 and Δcer1+CER1 at 37° C., 5% CO₂, pH 4.0 (intracellular). Data represented as mean±SEM.

FIG. 4B: In vitro growth of WT, Δcer1 and Δcer1+CER1 at 37° C., 5% CO₂, pH 7.4 (extracellular). Data represented as mean±SEM.

FIG. 4C: In vitro growth of WT and Δcer1 in YPD, 30° C., 0.04% CO₂. Data represented as mean±SEM.

FIG. 4D: Serial dilutions of WT, Δcer1, Δcer1+CER1, Δcer2, Δcer3 on solid YPD media supplemented with SDS or H₂O₂.

FIG. 4E: Transmission electron microscopy images of WT, Δcer1, and Δcer1 supplemented with ceramide mixture (Matreya LLC). Bar=500 nm.

FIG. 4F: Pma1 proton pump activity of WT, Δcer1, Δcer1+CER1, and Δcer1+C18 ceramide (Avanti Polar lipids, Alabaster, Ala.) measured by glucose dependent medium acidification. Data represented as mean±SEM.

FIG. 5A: Pma1 proton pump activity of WT, Δcer1, Δgcs1, Δgcs1+AbA, Δgcs1+AbA+C6 phytoceramide, and Δgcs1+AbA+C18 ceramide measured by glucose dependent medium acidification. Data represented as mean±SEM.

FIG. 5B: In vitro growth of WT, GAL7::IPC1, GAL7::IPC1+C6 phytoceramide, GAL7::IPC1+C18 ceramide, and Δcer1 at 37° C., 5% CO₂, pH 4.0. Data represented as mean±SEM.

FIG. 6A: General strategy for the deletion of ceramide synthases in C. neoformans wild-type (WT) and creation of the mutant strain Δcer.

FIG. 6B: Strategy for the generation of the complemented strain Δcer+CER.

FIG. 6C: Southern blot analysis for confirmation of transformants of Δcer1, Δcer2 and Δcer3. Lanes: 1-4 5′UTR probe for Δcer1. Lane 1—1 kb marker, lane 2—WT Cn, lane 3— Δcer1+CER1, lane 4—Δcer1. Lanes (6-8) gene probe for Δcer1 selection. 6—WT, 7—Δcer1, 8—Δcer1+CER1. Lane (9-16) 5′UTR and gene probes for Δcer2. 9—1 kb marker, 10—WT band, 11, 13—Δcer2 transformants, 12—WT band. Lanes (17-23) 5′UTR and gene probes for Δcer3. 17—1 kb marker, 18—WT band, 19—1cer3, 20—WT, 21—Δcer3, 22, 23—negative transformants. 5′ UTR, 5′ untranslated region; 3′ UTR, 3′ untranslated region; NAT1, nourseothricin 1; Cer1, ceramide synthase1 1; HYG. Hygromycin B.

FIG. 6D: Alignment of amino acid sequences of fungal and human ceramide synthases using Clustal Omega algorithm ‘*’ indicated conserved residues. Grey boxes are conserved residues that have been reported to be important for ceramide synthase activity. Sc, S. cereviaise; Ca, C. albicans; An, A. nidulans; Hu, Homo sapiens; Cn, C. neoformans.

FIG. 7A: Histopathology of Brain sections of mice infected intranasally with WT (at death) or Δcer1 (day 60). Sections stained with haematoxylin & Eosin. Bar=1 mm (left), 100 μm (right).

FIG. 7B: Histopathology of lungs obtained from CBA/J mice infected intranasally with wildtype (WT) or Δcer1 at days 1, 3 and 5 post infection. Infection with WT Cn shows a progression of inflammation along with replication of cells from day 1-5. Infection with Δcer1 shows a reduction in the number of cells from day 1-5 and cells start showing an elongated phenotype within 24-48 hours in the lung. Inflammation is observed during this time. Sections stained with Periodic acid Schiff's stain/Alcian Blue and Haematoxylin. Bar=20 μm. Black arrows show Cn cells.

FIG. 8A: Western blot using microsomal protein from Cer1 expressed in 2% glucose or 2% galactose. using anti-6×His antibody. 150 μg microsomal protein was used in each lane.

FIG. 8B: Ceramide synthase assay confirming activity of Cer1 in Sc. 150 μg microsomal protein was used in each lane.

FIG. 8C: Cer1 activity is temperature dependent. Ceramide synthase assay using 150 μg microsomal protein of Cer1 at temperature 28° C., 30° C., 35° C., and 37° C. Formation of NBD-ceramide was detected by thin layer chromatography. Mammalian Cer1 microsomal protein was used as a positive control.

FIG. 9A: Heat map of the sphingolipid profile for ceramide synthase deletion strains. The amount of lipid species are represented as relative abundance to corresponding WT lipid values. Blue bars represent lipid amount higher than WT. Green bars represent amount of lipid lower than WT. White bars are equal to WT values. Lipid profile at pH 4.0/intracellular conditions.

FIG. 9B: Lipid profile at pH 7.4/extracellular conditions. The scale is log₂.

FIG. 9C: Heat map of the sphingolipid profile for Δcer1, and Δcer1+CER1 strains. Lipid profile at pH 4.0/intracellular conditions.

FIG. 9D: Lipid profile at pH 7.4/extracellular conditions. The scale is log 2.

FIG. 10A: Replicative lifespan studies for WT, Δcer1, and Δcer1+CER1 shows that deletion of Cer1 leads to a drastic reduction of lifespan to an average 6.5 generations. Conversely, WT and Δcer1+CER1 has a lifespan of average 27 generations.

FIG. 10B: Light microscopy reveals cell wall defects in Δcer1.

FIG. 11: In vitro ceramide synthase assay. Thin Layer Chromatography of lipids after an in vitro enzymatic assay consisting of fluorescent sphingosine (NBD-sphingosine), palminate-CoA and microsomal preparation of either human ceramide synthase 1 (Hu Cer1) expressed in mammalian cells or Cryptococcus neoformans Cer1 (Cn Cer1) expressed under a galatose-inducible promoter in the model organism Saccharomyces cerevisiaie. Results show the production of NBD-ceramide only when Hu Cer1 or Cn Cer 1 expressed in galactose are used. Proper negative controls are included.

FIG. 12: Z′ Score calculation. Z score was determined by analyzing NBD ceramide formation in absence, negative control (NC) and in the presence of the enzyme Cn Cer1, positive control (PC).

FIG. 13: Transmission electron microscopy of ceramide synthase deletion mutants

FIG. 14: Phenotypic analysis of ceramide synthase deletion mutants.

FIG. 15: Brain enlargement of Intravenous Δcer1S1 infected mice and India ink staining of brain homogenate.

FIG. 16: Histological differences in lung tissue of CBA/J mice infected with WT (A, B, E, and F) and Δ67 (C, D, G, and H) C. neoformans. Sections were stained with mucicarmine (A, B, C, and D) and Haematoxylin and Eosin (E, F, G, and H). With mucicarmine staining no cryptococcal cells are found in Δ67 infected lung at day 60 post infection while WT cells are abundantly observed. Infection with Δ67 causes no inflammation and open alveolar spaces while infection with WT shows strong inflammation and damage to lung tissue. WT tissue samples collected at day 15 post infection.

FIG. 17: Intravenous infection with CnCerS1. First row: histology of brain at day 12, stained with mucicarmine; Middle row: histology of brain at day 12, stained with Haematoxylin and Eosin; third row: histology of lung at day 12, stained with mucicarmine.

FIG. 18: Survival and immunization studies of ceramide synthase deletion mutants in murine animal model, showing pre-treatment, WT challenge, and days post challenge.

FIG. 19: Survival and immunization studies of ceramide synthase deletion mutants in murine animal model, highlighting the days post-infection.

FIG. 20: pH as a function of FA CoA Chain Length.

FIG. 21: Overexpression and characterization of cryptococcal ceramide synthase.

FIG. 22: Assay showing pH dependence of ceramide synthase enzyme activity.

FIG. 23: Relative activity of ceramide synthase enzyme at different pH values.

FIG. 24: Phylogenetic Tree.

FIG. 25: Alignments of fungal and mammalian ceramide synthases.

### DETAILED DESCRIPTION OF THE INVENTION

The present invention provides a method of inhibiting the growth of a fungus comprising contacting the fungus with an effective amount of an inhibitor so as to thereby inhibit the growth of the fungus,
wherein the inhibitor inhibits ceramide synthase 1 (Cer1) in the fungal cells of the fungus.

The present invention provides a method of treating a subject afflicted with a fungal infection comprising administering to the subject an effective amount of an inhibitor so as to treat the subject afflicted with the fungal infection,
wherein the inhibitor inhibits ceramide synthase 1 (Cer1) in the fungal cells of the fungus.

In some embodiments, the method wherein the inhibitor inhibits Cer1 activity or inhibits Cer1 expression.

In some embodiments, the method wherein the inhibitor inhibits Cer1 without substantially inhibiting a human ceramide synthase.

In some embodiments, a method of inhibiting fungal ceramide synthase 1 (Cer1) activity comprising contacting the Cer1 with an effective amount of an inhibitor.

In some embodiments, the method wherein the Cer1 is in a fungal cell.

In some embodiments, the method wherein the inhibitor inhibits fungal synthesis of ceramides and/or glucosylceramides.

In some embodiments, the method wherein the fungus is Cryptococcus neoformans, Blastomyces dermatitidis, Cryptococcus gattii, Candida albicans, Candida auris, Candida krusei, Candida glabrata, Candida parapsilosis, Candida guilliermondii, Coccidioides immitis, Aspergillus fumigatus, Pichia kudriavzevii, Rhizopus oryzae, Rhizopus spp., Histoplasma capsulatum, Coccidioides spp., Paecilomyces variotii, Pneumocystis murina, Pneumocystis jiroveci, Scedosporium spp., Sporotrix spp. Aspergillus spp., a dimorphic fungi or a mucorales fungi.

In some embodiment, the subject is infected with a fungal infection of Cryptococcus neoformans, Blastomyces dermatitidis, Cryptococcus gattii, Candida albicans, Candida auris, Candida krusei, Candida glabrata, Candida parapsilosis, Candida guilliermondii, Coccidioides immitis, Aspergillus fumigatus, Pichia kudriavzevii, Rhizopus oryzae, Rhizopus spp., Histoplasma capsulatum, Coccidioides spp., Paecilomyces variotii, Pneumocystis murina, Pneumocystis jiroveci, Scedosporium spp., Sporotrix spp. Aspergillus spp., a dimorphic fungi or a mucorales fungi.

In some embodiments, the method wherein the inhibitor is a small molecule, a synthetic small molecule, a peptide, a protein, an anti-sense oligonucleotide or an RNA molecule.

In some embodiments, the method wherein wherein the inhibitor comprises a CRISPR nuclease.

In some embodiments, the method wherein the inhibitor comprises a CRISPR nuclease; and a gRNA or sgRNA.

In some embodiments, the method wherein the inhibitor comprises a CRISPR nuclease; an RNA guide molecule; and a tracrRNA.

In some embodiments, a method for inhibiting expression of a fungal ceramide synthase 1 (Cer1) in a fungal cell, the method comprising delivering to the fungal cell an RNA molecule, thereby inhibiting expression of the Cer1.

In some embodiments, the method wherein the RNA molecule is siRNA, shRNA, dsRNA, gRNA or sgRNA molecule.

In some embodiments, the method wherein the RNA molecule comprises a sequence that is complementary to a sequence in the target fungal Cer1 gene.

In some embodiments, the method wherein the inhibitor is a small molecule.

In some embodiments, the method wherein the inhibitor is a synthetic small molecule.

In some embodiments, the method further comprising contacting the fungus with an anti-fungal agent.

In some embodiments, the method further comprising administering an anti-fungal agent to the subject.

In some embodiments, a method for inhibiting expression of a fungal ceramide synthase 1 (Cer1) in a fungal cell, the method comprising delivering to the fungal cell:
a CRISPR nuclease;
an RNA guide molecule;
and a tracrRNA,
wherein RNA molecule comprises a sequence that is complementary to a sequence in the target fungal Cer1 gene.

In some embodiments, a method for inhibiting expression of a fungal ceramide synthase 1 (Cer1) in a fungal cell, the method comprising delivering to the fungal cell a CRISR nuclease that targets a sequence of the Cer1 gene, thereby inhibiting expression of the fungal ceramide synthase 1 (Cer1).

In some embodiments, a method of identifying an agent that inhibits the growth of a fungus comprising:
(i) determining whether the agent inhibits fungal ceramide synthase 1 (Cer1),
wherein the presence of fungal ceramide synthase 1 (Cer1) inhibitory activity identifies the agent which inhibits the growth of the fungus.

In some embodiments, the method further comprising:
(i) determining whether the agent inhibits a human ceramide synthase,
wherein the presence of fungal ceramide synthase 1 (Cer1) inhibitory activity and the absence of substantial human ceramide synthase inhibitory activity identifies the agent which inhibits the growth of the fungus in the human subject.

In some embodiments, a method of identifying an antagonist of fungal ceramide synthase 1 (Cer1) comprising:
(i) contacting a fungal cell which expresses the Cer1 with an agent, and
(ii) determining whether said agent inhibits the Cer1,
wherein an agent that inhibits the Cer1 is an antagonist of the Cer1.

The present invention also provides an inhibitor of fungal ceramide synthase 1 (Cer1) activity.

In some embodiments, wherein the inhibitor is a small molecule or a synthetic small molecule.

In some embodiments, wherein the inhibitor is a peptide or protein.

In some embodiments, wherein the inhibitor acts directly on fungal ceramide synthase 1.

In some embodiments, wherein the inhibitor acts downstream of fungal ceramide synthase 1.

In some embodiments, wherein the inhibitor acts upstream of fungal ceramide synthase 1.

In some embodiments, wherein the inhibitor targets a polypeptide or protein comprising or consisting of SEQ ID NO: 9.

In some embodiments, wherein the inhibitor is an anti-sense oligonucleotide.

In some embodiments, wherein the inhibitor is an RNA molecule.

In some embodiments, wherein the inhibitor is an siRNA, shRNA, dsRNA, gRNA or sgRNA molecule

In some embodiments, wherein the inhibitor comprises a CRISPR nuclease.

In some embodiments, wherein the inhibitor comprises a CRISPR nuclease and a gRNA or sgRNA.

In some embodiments, wherein the inhibitor comprises a CRISPR nuclease; an RNA guide molecule; and a tracrRNA.

In some embodiments, the inhibitor further comprising a gene knockout cassette.

In some embodiments, the inhibitor wherein the nucleotide sequence of the RNA, siRNA, shRNA, dsRNA, gRNA, or sgRNA molecule comprises or consists of a nucleotide sequence as set forth in SEQ ID NO: 1, a nucleotide sequence complementary to the nucleotide sequence as set forth in SEQ ID NO: 1, or a nucleotide sequence lacking one or more nucleotides from the 5′ end of SEQ ID NO: 1.

In some embodiments, the inhibitor wherein the inhibitor inhibits Cer1 activity or Cer1 expression.

In some embodiments, a method of identifying an agent that inhibits the activity of fungal ceramide synthase 1 (Cer1) comprising:
(i) contacting the Cer1 with the agent and separately with the compound of claim 39 or salt thereof; and
(ii) comparing the Cer1 inhibitory activity of the agent with the Cer1 inhibitory activity of the compound to identify the agent with Cer1 inhibitory activity that is greater than that of the compound.

In any one of the embodiments of the above methods or inhibitors, the small molecule has the structure:

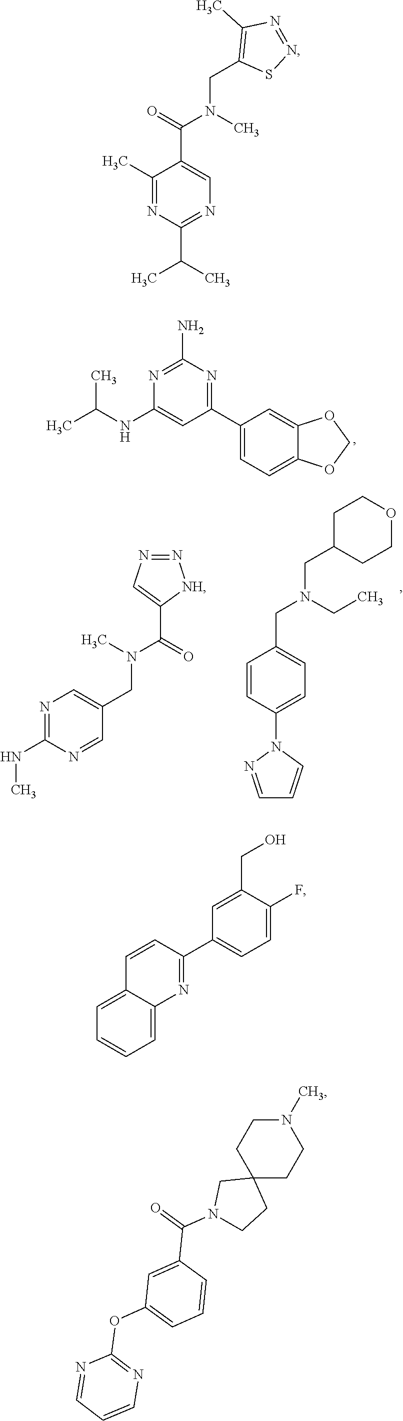

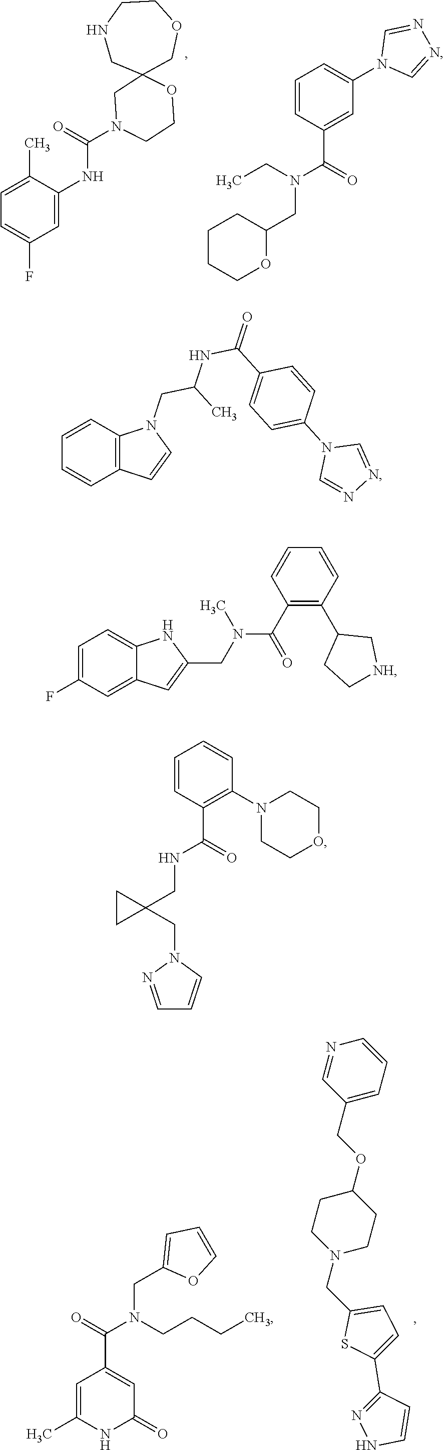

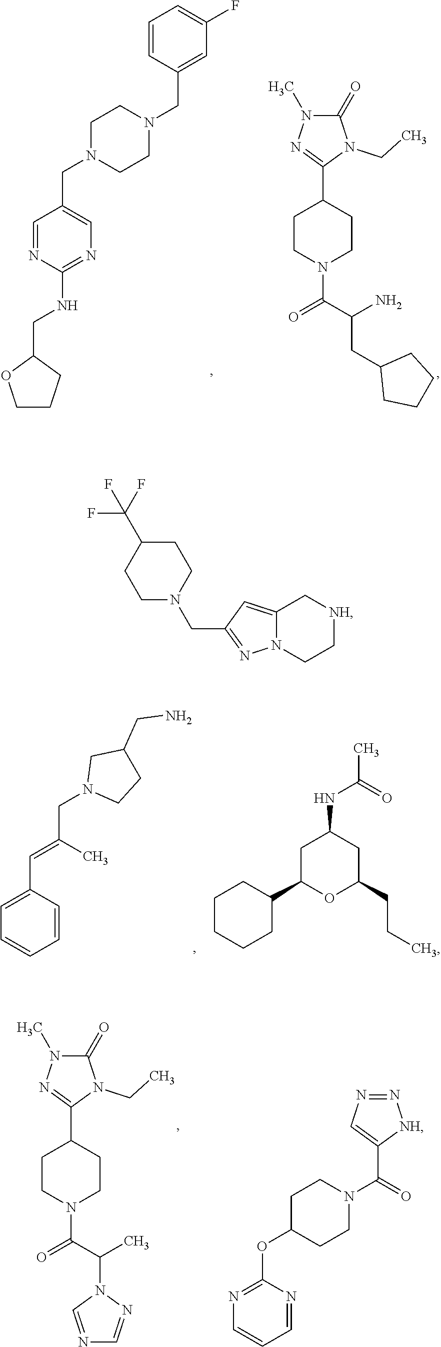

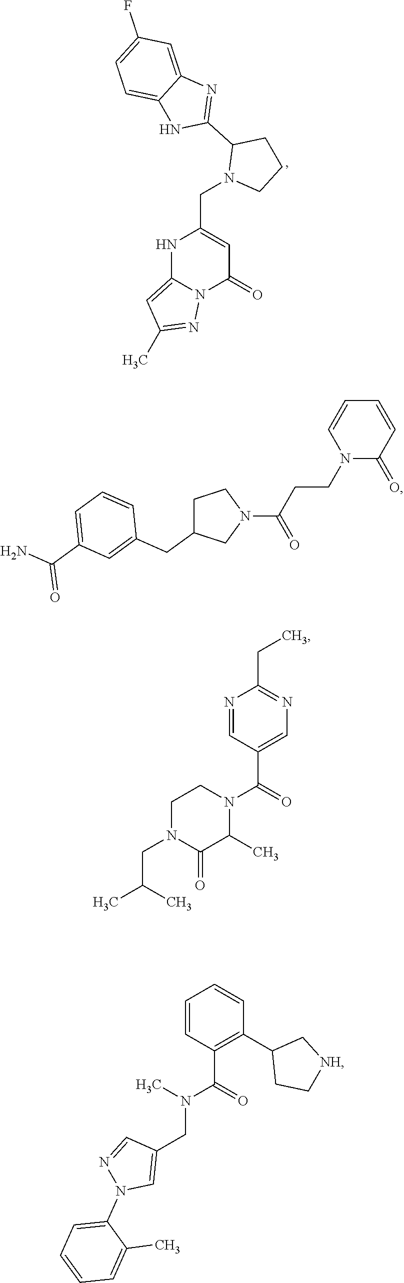 [SMILES: Cc1cc2[nH]c(CN3CCCC3c3nc4cc(F)ccc4[nH]3)cc(=O)n2n1 ; NC(=O)c1cccc(CC2CCN(C(=O)CCn3ccccc3=O)C2)c1 ; CCc1ncc(C(=O)N2CCN(CC(C)C)C(=O)C2C)cn1 ; Cc1ccccc1-n1cc(CN(C)C(=O)c2ccccc2C2CCNC2)cn1]

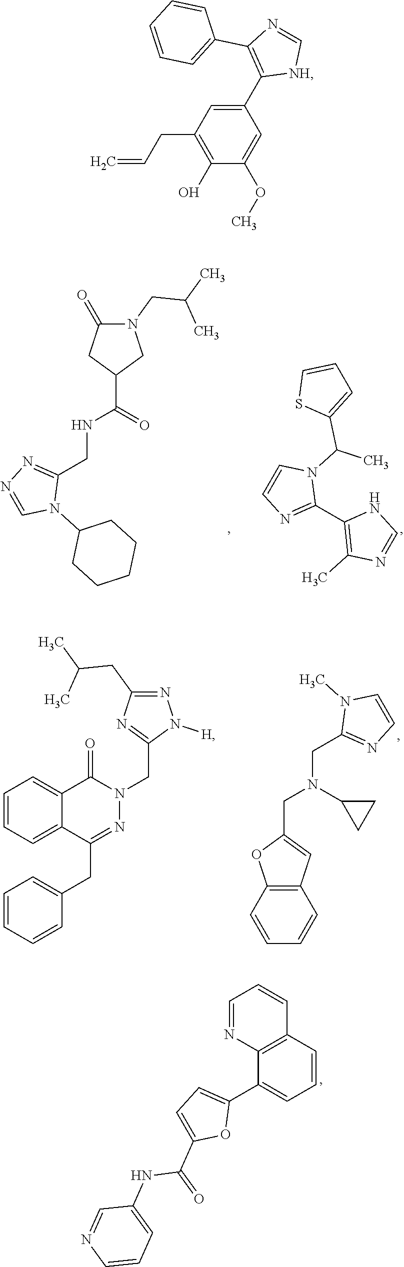

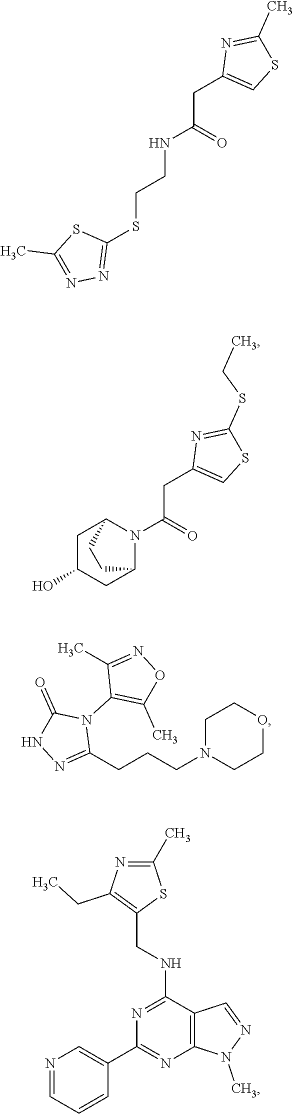 [SMILES: Cc1nc(CC(=O)NCCSc2nnc(C)s2)cs1 ; CCSc1nc(CC(=O)N2[C@@H]3CC[C@H]2C[C@@H](O)C3)cs1 ; Cc1noc(C)c1-n1c(CCCN2CCOCC2)n[nH]c1=O ; CCc1nc(C)sc1CNc1nc(-c2cccnc2)nc2c1cnn2C]

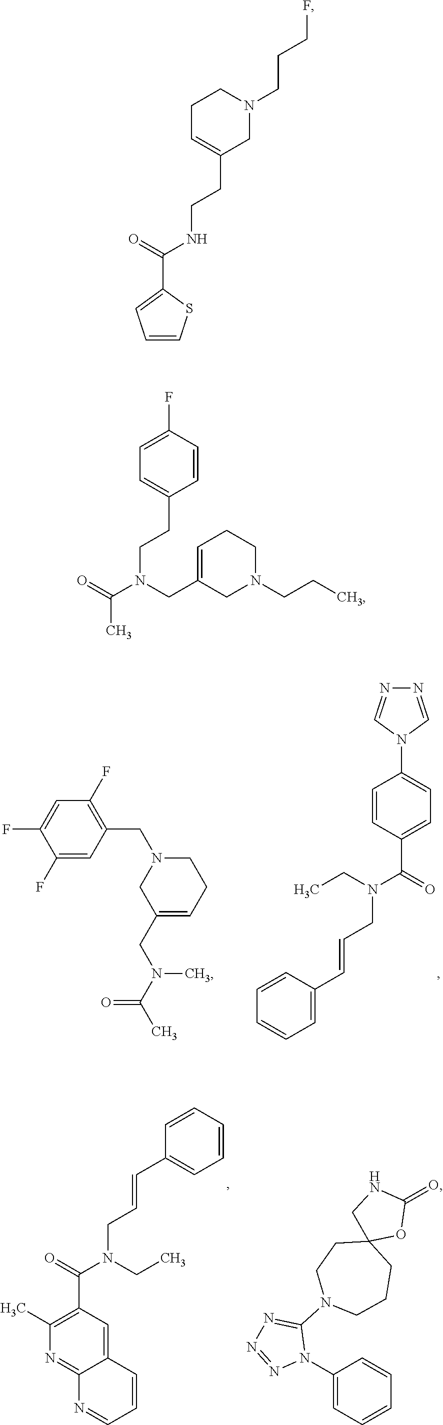 [SMILES: O=C(NCCC1=CCCN(CCCF)C1)c1cccs1 ; CCCN1CCC=C(CN(CCc2ccc(F)cc2)C(C)=O)C1 ; CC(=O)N(C)CC1=CCCN(Cc2cc(F)c(F)cc2F)C1 ; CCN(C/C=C/c1ccccc1)C(=O)c1ccc(-n2cnnc2)cc1 ; CCN(C/C=C/c1ccccc1)C(=O)c1cc2cccnc2nc1C ; O=C1NCC2(CCCN(c3nnnn3-c3ccccc3)CC2)O1]

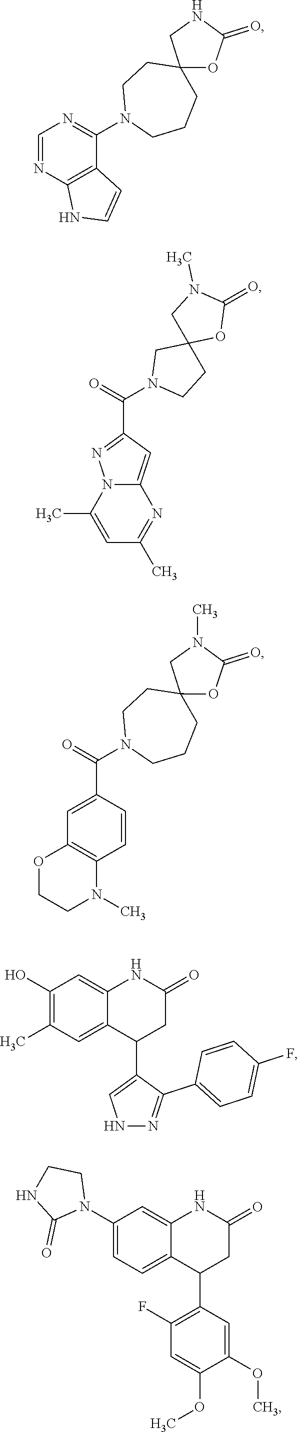 [SMILES: O=C1NCC2(CCCN(c3ncnc4[nH]ccc34)CC2)O1 ; Cc1cc(C)n2nc(C(=O)N3CCC4(CN(C)C(=O)O4)C3)cc2n1 ; CN1CC2(CCCN(C(=O)c3ccc4c(c3)OCCN4C)CC2)OC1=O ; Cc1cc2c(cc1O)NC(=O)CC2c1c[nH]nc1-c1ccc(F)cc1 ; COc1cc(F)c(C2CC(=O)Nc3cc(N4CCNC4=O)ccc32)cc1OC]

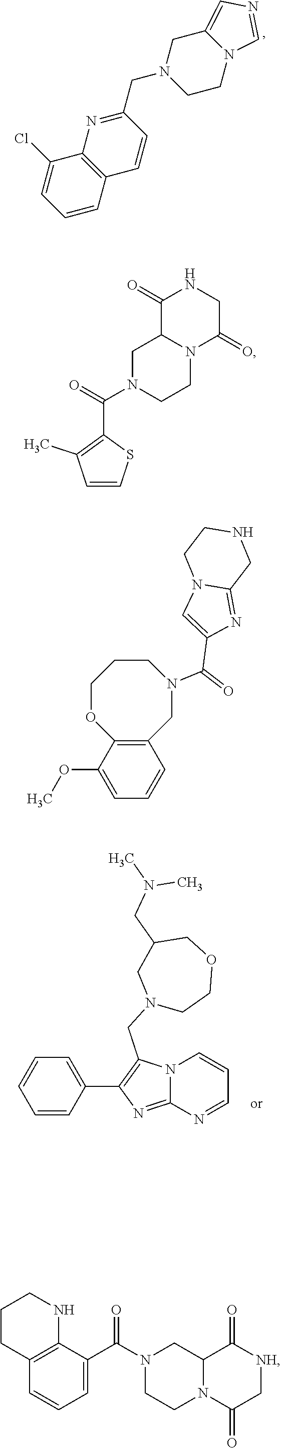 [SMILES: Clc1cccc2ccc(CN3CCn4cncc4C3)nc12 ; Cc1ccsc1C(=O)N1CCN2C(=O)CNC(=O)C2C1 ; COc1cccc2c1OCCCN(C(=O)c1cn3c(n1)CNCC3)C2 ; CN(C)CC1COCCN(Cc2c(-c3ccccc3)nc3ncccn23)C1 ; O=C1NCC(=O)N2CCN(C(=O)c3cccc4c3NCCC4)CC12]

or a salt thereof.

In any one of the embodiments of the above methods or inhibitors, the small molecule has the structure of any of the following compounds:

In some embodiments, the compound having the structure:

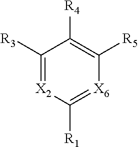

X2 is CR2 or N,
wherein R2 is H, halogen, OH, NH₂, CF₃, OCF₃, OCHF₂, CN, NO₂, alkyl, haloalkyl, alkenyl, alkynyl, alkoxy, haloalkoxy, aryloxy, heteroaryloxy, alkylamino, R₇O-alkyl, R₇S-alkyl, R₈R₉N-alkyl, CO₂R₈, C(O)NHR₈, C(O)NR₈R₉, NR₈R₉, C(O)CH₂OR₈, aryl, substituted aryl, heteroaryl or substituted heteroaryl;

X6 is CR6 or N,
wherein R6 is H, halogen, OH, NH₂, CF₃, OCF₃, OCHF₂, CN, NO₂, alkyl, haloalkyl, alkenyl, alkynyl, alkoxy, haloalkoxy, aryloxy, heteroaryloxy, alkylamino, R₇O-alkyl, R₇S-alkyl, R₈R₉N-alkyl, CO₂R₈, C(O)NHR₈, C(O)NR₈R₉, NR₈R₉, C(O)CH₂OR₈, aryl, substituted aryl, heteroaryl or substituted heteroaryl;

Each of R1, R3 and R5 is, independently, H, halogen, OH, NH₂, CF₃, OCF₃, OCHF₂, CN, NO₂, alkyl, haloalkyl, alkenyl, alkynyl, alkoxy, haloalkoxy, aryloxy, heteroaryloxy, alkylamino, R₇O-alkyl, R₇S-alkyl, R₈R₉N-alkyl, CO₂R₈, C(O)NHR₈, C(O)NR₈R₉, NR₈R₉, C(O)CH₂OR₈, aryl, substituted aryl, heteroaryl or substituted heteroaryl; and

R4 is H, halogen, OH, NH₂, CF₃, OCF₃, OCHF₂, CN, NO₂, alkyl, haloalkyl, alkenyl, alkynyl, alkoxy, haloalkoxy, aryloxy, heteroaryloxy, alkylamino, R₇O-alkyl, R₇S-alkyl, R₈R₉N-alkyl, R₉C(O) NR₈-alkyl, CO₂R₇, C(O) NHR₈, C(O)NR₈R₉, NR₈R₉, NHC(O)NR₈R₉, C(O)CH₂OR₇, aryl, substituted aryl, heteroaryl or substituted heteroaryl,
wherein each R7 is independently H, alkyl, alkenyl, alkynyl, arylalkyl, aryl or heteroaryl;
wherein each R8 and R9 is, independently, H, alkyl, alkenyl, alkynyl, arylalkyl, heteroarylalkyl, heterocycloalkylalkyl, aryl or heteroaryl, or R8 and R9 combine to form a cycloalkyl or heterocycloalkyl,

or a pharmaceutically acceptable salt thereof.

In some embodiments, the compound wherein X2 and X6 are both N.

In some embodiments, the compound wherein X2 is CR2 and X6 is CR6.

In some embodiments, the compound wherein

X2 is CR2 or N,
wherein R2 is H, halogen, OH, NH₂, CF₃, OCF₃, OCHF₂, CN, NO₂, alkyl, haloalkyl, alkenyl, alkynyl, alkoxy, haloalkoxy, aryloxy, heteroaryloxy, alkylamino or R₇O-alkyl,

X6 is CR6 or N,
wherein R6 is H, halogen, OH, NH₂, CF₃, OCF₃, OCHF₂, CN, NO₂, alkyl, haloalkyl, alkenyl, alkynyl, alkoxy, haloalkoxy, aryloxy, heteroaryloxy, alkylamino or R₇O-alkyl,

Each of R1, R3 and R5 is, independently, H, halogen, OH, NH₂, CF₃, OCF₃, OCHF₂, CN, NO₂, alkyl, haloalkyl, alkenyl, alkynyl, alkoxy, haloalkoxy, aryloxy, heteroaryloxy, alkylamino, aryl, or heteroaryl; and

R4 is H, NR₈R₉N-alkyl, R₉C(O) NR₈-alkyl, CO₂R₇, C(O) NHR₈, C(O) NR₈R₉, NR₈R₉, NHC(O)NR₈R₉, aryl, substituted aryl, heteroaryl or substituted heteroaryl,
wherein each R7 is independently H, alkyl, alkenyl, alkynyl, arylalkyl, aryl or heteroaryl;
wherein each R8 and R9 is, independently, H, alkyl, alkenyl, alkynyl arylalkyl, heteroarylalkyl, heterocycloalkylalkyl, aryl or heteroaryl, or R8 and R9 when attached to the same N combine to form a cycloalkyl or heterocycloalkyl,

or a pharmaceutically acceptable salt thereof.

In some embodiments, the compound wherein

X2 is CR2 or N,
wherein R2 is H, halogen, heteroaryloxy or R₇O-alkyl,

X6 is CR6 or N,
wherein R6 is H, halogen, heteroaryloxy or R₇O-alkyl,

Each of R1, R3 and R5 is, independently, H, halogen, NH₂, alkyl, alkylamino, aryl, or heteroaryl; and

R4 is H, R₈R₉N-alkyl, R₉C(O)NR₈-alkyl, CO₂R₇, C(O)NHR₈, C(O)NR₈R₉, NR₈R₉, NHC(O)NR₈R₉, aryl or heteroaryl;
wherein each R7 is independently H, alkyl, alkenyl, alkynyl, alkylaryl, aryl or heteroaryl;
wherein each R8 and R9 is, independently, H, alkyl, alkenyl, alkynyl, arylalkyl, heteroarylalkyl, heterocycloalkylalkyl, aryl or heteroaryl, or R8 and R9 when attached to the same N combine to form a cycloalkyl or heterocycloalkyl,

or a pharmaceutically acceptable salt thereof.

In some embodiments, the compound wherein

X2 is CR2 or N,
wherein R2 is H, halogen, heteroaryloxy or R₇O-alkyl,

X6 is CR6 or N,
wherein R6 is H, halogen, heteroaryloxy or R₇O-alkyl,

Each of R1, R3 and R5 is, independently, H, halogen, NH₂, alkyl, alkylamino, aryl, or heteroaryl; and

R4 is H, R₈R₉N-alkyl, R₉C(O) NR₈-alkyl, CO₂R₇, C(O) NHR₈, C(O) NR₈R₉, NR₈R₉, NHC(O)NR₈R₉, aryl or heteroaryl;
wherein each R7 is independently H, alkyl, alkenyl, alkynyl, alkylaryl, aryl or heteroaryl;
wherein each R8 and R9 is, independently, H, alkyl, alkenyl, alkynyl, arylalkyl, heteroarylalkyl, heterocycloalkylalkyl, aryl or heteroaryl, or R8 and R9 when attached to the same N combine to form a cycloalkyl or heterocycloalkyl,

or a pharmaceutically acceptable salt thereof.

In some embodiments, the compound having the structure:

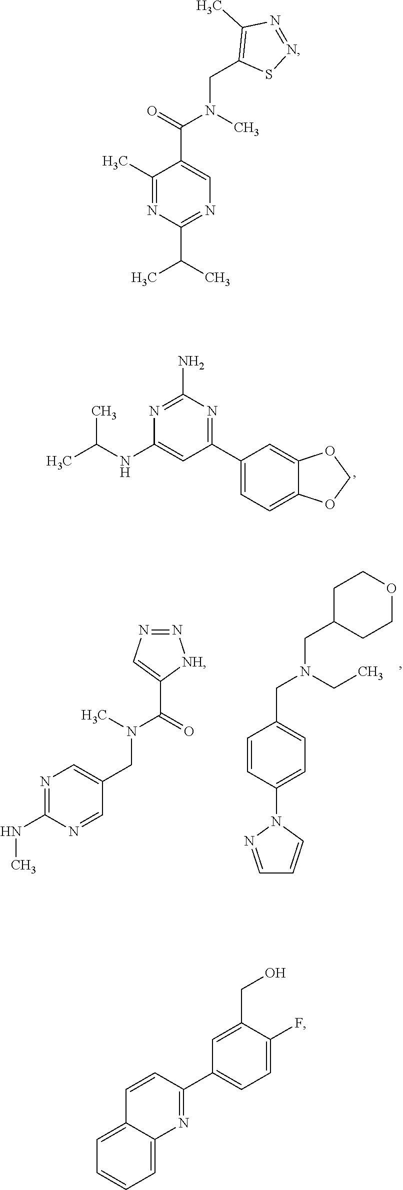 [SMILES: Cc1nc(C(C)C)ncc1C(=O)N(C)Cc1snnc1C ; CC(C)Nc1cc(-c2ccc3c(c2)OCO3)nc(N)n1 ; CNc1ncc(CN(C)C(=O)c2cnn[nH]2)cn1 ; CCN(Cc1ccc(-n2cccn2)cc1)CC1CCOCC1 ; OCc1cc(-c2ccc3ccccc3n2)ccc1F]

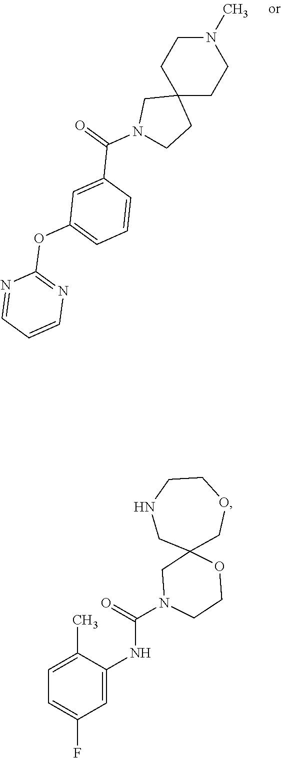 [SMILES: CN1CCC2(CC1)CCN(C(=O)c1cccc(Oc3ncccn3)c1)C2 ; Cc1ccc(F)cc1NC(=O)N1CCOC2(CNCCOC2)C1]

or a pharmaceutically acceptable salt thereof.

In some embodiments, the compound having the structure:

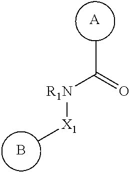

A is a substituted or unsubstituted aryl, heteroaryl or lactam;

B is a substituted or unsubstituted aryl, heteroaryl or heterocycloalkyl;

R1 is H, alkyl, haloalkyl, alkenyl or alkynyl; and

X1 is present or absent, and when present is an alkyl, cycloalkyl or alkenyl linker;

or a pharmaceutically acceptable salt thereof.

In some embodiments, the compound wherein X1 is present and has the structure:

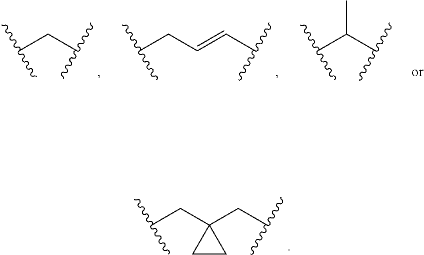

In some embodiments, the compound wherein A is a substituted or unsubstituted lactam, phenyl, pyridine, pyrimidine, pyrazine, indole, isoindole, benzofuran, benzothiophene, indazole, benzimidazole, benzthiazole, quinoline, naphthyridine or isoquinoline.

In some embodiments, the compound wherein A has structure:

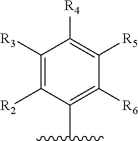

wherein

each of R2, R3, R4, R5 and R6 is, independently, H, halogen, OH, CF₃, OCF₃, OCHF₂, CN, NO₂, alkyl, haloalkyl, alkenyl, alkynyl, alkoxy, haloalkoxy, alkylamino, aryl, substituted aryl, heteroaryl or substituted heteroaryl.

In some embodiments, the compound wherein A has structure:

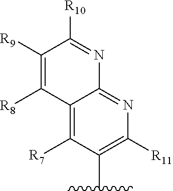

wherein

each of R7, R8, R9, R10 and R11 is, independently, H, halogen, OH, CF₃, OCF₃, OCHF₂, CN, NO₂, alkyl, haloalkyl, alkenyl, alkynyl, alkoxy, haloalkoxy, alkylamino, aryl, substituted aryl, heteroaryl or substituted heteroaryl.

In some embodiments, the compound wherein A has structure:

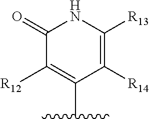

wherein

each of R12, R13 and R14 is, independently, H, halogen, OH, CF₃, OCF₃, OCHF₂, CN, NO₂, alkyl, haloalkyl, alkenyl, alkynyl, alkoxy, haloalkoxy, alkylamino, aryl, substituted aryl, heteroaryl or substituted heteroaryl.

In some embodiments, the compound wherein B is a substituted or unsubstituted pyrazole, furan, tetrahydropyran, phenyl, pyridine, pyrimidine, pyrazine, indole, isoindole, benzofuran, benzothiophene, indazole, benzimidazole, benzthiazole, quinoline, naphthyridine or isoquinoline.

In some embodiments, the compound wherein B has structure:

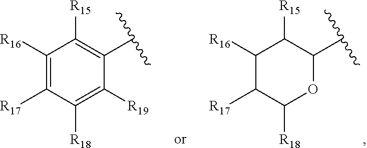

wherein

each of R15, R16, R17, R18 and R19 is, independently, H, halogen, OH, CF₃, OCF₃, OCHF₂, CN, NO₂, alkyl, haloalkyl, alkenyl, alkynyl, alkoxy, haloalkoxy, alkylamino, aryl, substituted aryl, heteroaryl or substituted heteroaryl.

In some embodiments, the compound wherein B has structure:

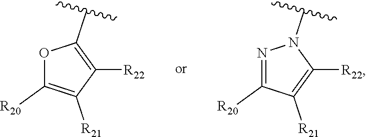

wherein

each of R20, R22 and R22 is, independently, H, halogen, OH, CF₃, OCF₃, OCHF₂, CN, NO₂, alkyl, haloalkyl, alkenyl, alkynyl, alkoxy, haloalkoxy, alkylamino, aryl, substituted aryl, heteroaryl or substituted heteroaryl.

In some embodiments, the compound wherein B has structure:

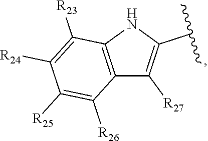

wherein

each of R23, R24, R25, R26 and R27 is, independently, H, halogen, OH, CF₂, OCF₃, OCHF₂, CN, NO₂, alkyl, haloalkyl, alkenyl, alkynyl, alkoxy, haloalkoxy, alkylamino, aryl, substituted aryl, heteroaryl or substituted heteroaryl.

In some embodiments, the compound wherein wherein R1 is methyl or ethyl.

In some embodiments, the compound wherein having the structure:

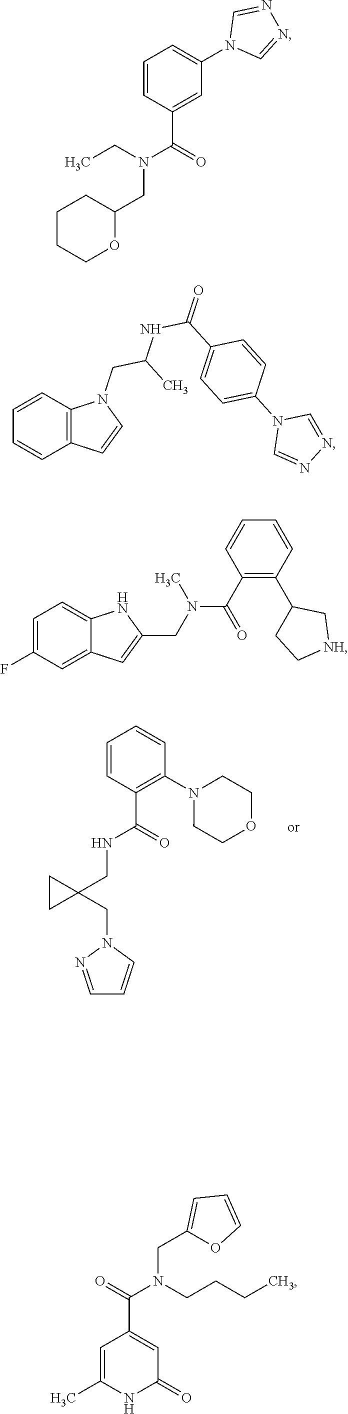 [SMILES: CCN(CC1CCCCO1)C(=O)c1cccc(-n2cnnc2)c1 ; CC(Cn1ccc2ccccc21)NC(=O)c1ccc(-n2cnnc2)cc1 ; CN(Cc1cc2cc(F)ccc2[nH]1)C(=O)c1ccccc1C1CCNC1 ; O=C(NCC1(Cn2cccn2)CC1)c1ccccc1N1CCOCC1 ; CCCCN(Cc1ccco1)C(=O)c1cc(C)[nH]c(=O)c1]

or a pharmaceutically acceptable salt thereof.

In some embodiments, the compound having the structure:

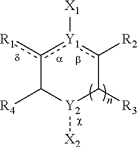

wherein

n=0 or 1;

α, β and χ are each a bond that is present or absent,
wherein both α and β are absent, or α is present and β is absent, or α is absent and β is present, and
wherein when Y2 is O, then bond χ is absent, and when Y2 is N, bond χ is present;

δ is a bond that is present or absent,
wherein when R1 is O, then bond δ is present, and when R1 is other than O, then bond δ is absent,

Y1 is C or N;

Y2 is O or N,
wherein when Y2 is O, then bond χ is absent, and when Y2 is N, bond χ is present;

X1 is H, halogen, OH, CF₃, OCF₃, OCHF₂, CN, NO₂, alkyl, hydroxyalkyl, aminoalkyl, NH(CO)-alkyl, haloalkyl, alkenyl, alkynyl, aryl, alkylaryl, heteroaryl, heteroarylalkyl, aryloxy, arylalkoxy, heteroaryloxy, heteroarylalkoxy, aryl-C(O)NH-alkyl, or heteroaryl-C(O)NH-alkyl,

X2 is alkyl, hydroxyalkyl, aminoalkyl, haloalkyl, alkenyl, alkynyl, aryl, arylalkyl, arylalkenyl, heteroaryl, heteroarylalkyl, aryloxy, arylalkoxy, heteroaryloxy, heteroarylalkoxy, biaryl, biheteroaryl, biarylalkyl, biheteroarylalkyl, alkyl-CO, aryl-C(O), heteroaryl-C(O), alkyl-NHC(O), cycloalkyl-NHC(O), lactam-alkyl-C(O), arylalkyl-C(O) or heteroarylalkyl-C(O),

R1 is O, or is H, halogen, OH, CF₃, OCF₃, OCHF₂, CN, NO₂, alkyl, hydroxyalkyl, aminoalkyl, NH(CO)-alkyl, haloalkyl, alkenyl, alkynyl, aryl, alkylaryl, heteroaryl, heteroarylalkyl, aryloxy, arylalkoxy, heteroaryloxy, heteroarylalkoxy, aryl-C(O)NH-alkyl, or heteroaryl-C(O)NH— alkyl; and

each of R2, R3 and R4 is, independently, H, halogen, OH, CF₃, OCF₃, OCHF₂, CN, NO₂, alkyl, hydroxyalkyl, aminoalkyl, NH(CO)-alkyl, haloalkyl, alkenyl, alkynyl, aryl, alkylaryl, heteroaryl, heteroarylalkyl, aryloxy, arylalkoxy, heteroaryloxy, heteroarylalkoxy, aryl-C(O)NH-alkyl, or heteroaryl-C(O)NH-alkyl,

or a pharmaceutically acceptable salt thereof.

In some embodiments, the compound having the structure:

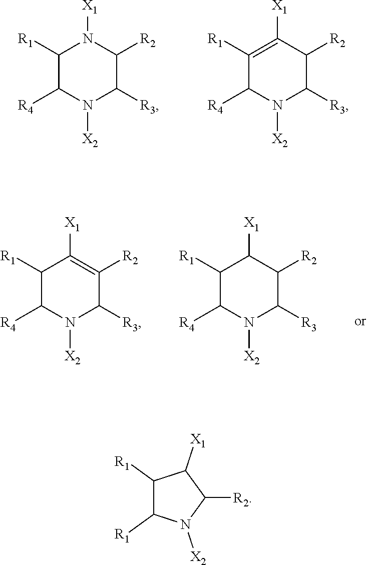

In some embodiments, the compound having the structure:

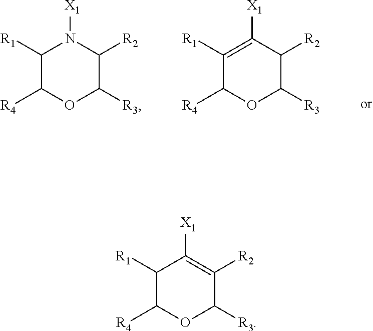

In some embodiments, the compound having the structure:

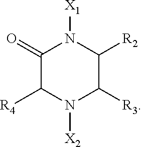

In some embodiments, the compound wherein

X1 is H, CF₃, alkyl, aryl, alkylaryl, heteroaryl, heteroarylalkyl, aryloxy, arylalkoxy, heteroaryloxy, heteroarylalkoxy, aryl-C(O)NH-alkyl, or heteroaryl-C(O)NH-alkyl,

X2 is alkyl, hydroxyalkyl, aminoalkyl, haloalkyl, aryl, arylalkyl, arylalkenyl, heteroaryl, heteroarylalkyl, aryloxy, arylalkoxy, heteroaryloxy, heteroarylalkoxy, biaryl, biheteroaryl, biarylalkyl, biheteroarylalkyl, alkyl-CO, aryl-C(O), heteroaryl-C(O), alkyl-NHC(O), cycloalkyl-NHC(O), lactam-alkyl-C(O), arylalkyl-C(O) or heteroarylalkyl-C(O),

each of R1, R2, R3 and R4 is, independently, H, CF₃, alkyl, H, CF₃, OCF₃, hydroxyalkyl, aminoalkyl, NH(CO)-alkyl, haloalkyl, alkenyl, alkynyl, aryl, alkylaryl, heteroaryl, heteroarylalkyl, aryloxy, arylalkoxy, heteroaryloxy, heteroarylalkoxy, aryl-C(O)NH-alkyl, or heteroaryl-C(O)NH— alkyl; and

or a pharmaceutically acceptable salt thereof.

In some embodiments, the compound wherein both X1 and X2 are other than H.

In some embodiments, the compound wherein X1 is H.

In some embodiments, the compound wherein one of R1-R4 is other than H.

In some embodiments, the compound having the structure:

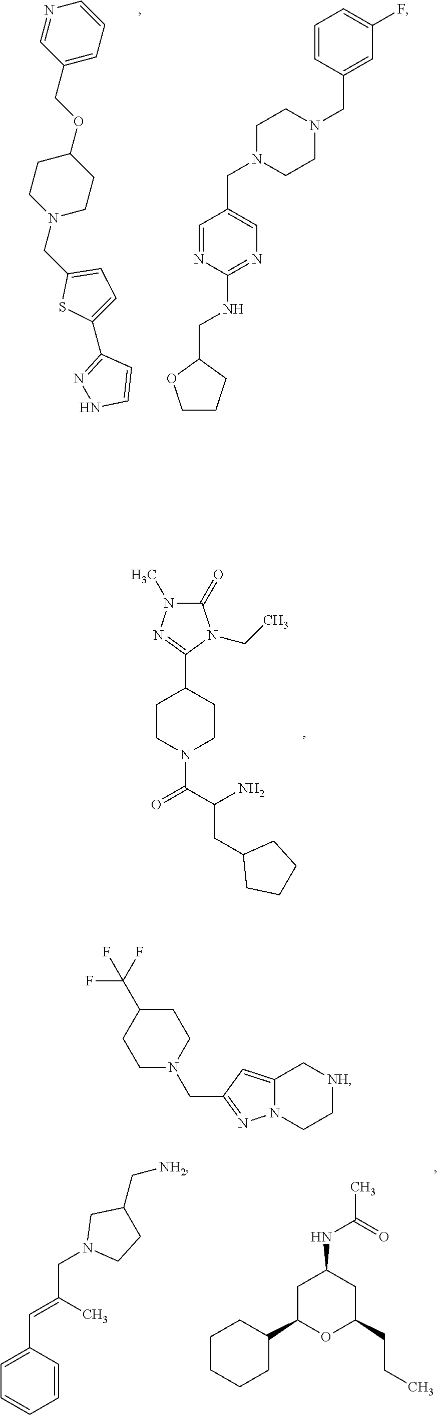

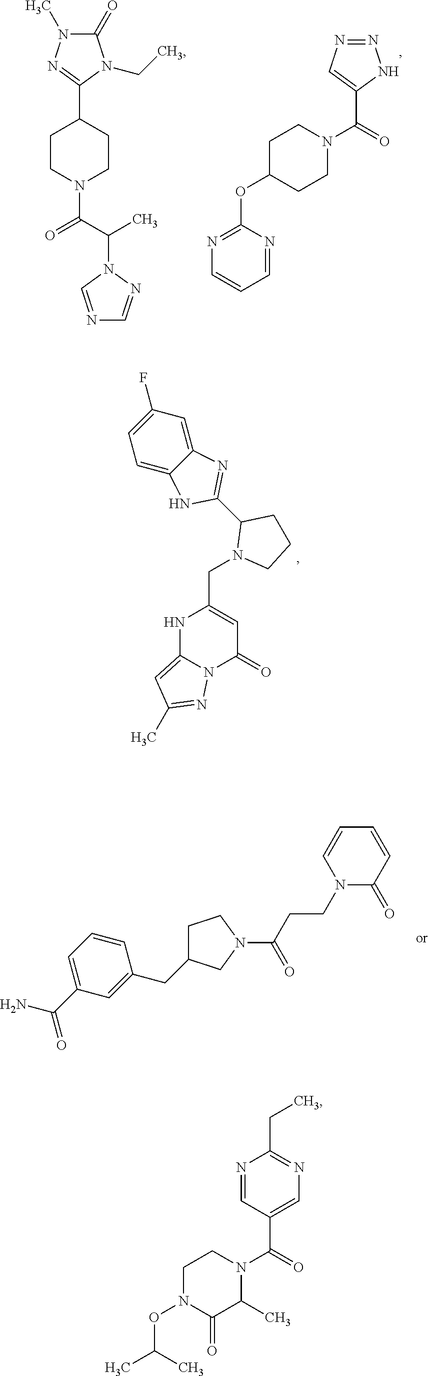 [SMILES: CCn1c(C2CCN(C(=O)C(C)n3cncn3)CC2)nn(C)c1=O ; O=C(c1cnn[nH]1)N1CCC(Oc2ncccn2)CC1 ; Cc1cc2[nH]c(CN3CCCC3c3nc4cc(F)ccc4[nH]3)cc(=O)n2n1 ; NC(=O)c1cccc(CC2CCN(C(=O)CCn3ccccc3=O)C2)c1 ; CCc1ncc(C(=O)N2CCN(OC(C)C)C(=O)C2C)cn1]

or a pharmaceutically acceptable salt thereof.

In some embodiments, the compound having the structure:

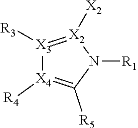

wherein

R1 is H, alkyl, alkenyl, alkynyl, cycloalkyl, alkylaryl, aryl, substituted aryl, heteroaryl or substituted heteroaryl;

Each of R2, R3, R4 and R5 is present or absent, and when present is H, halogen, alkyl, alkenyl, alkynyl, cycloalkyl, cycloheteroalkyl, substituted cycloheteroalkyl, aryl, substituted aryl, heteroaryl, substituted heteroaryl, alkyl-N(alkyl)(CO)-lactam or alkyl-N(alkyl)(CO)-(aryl),

X2 is C or N,
wherein when X2 is N, R2 is absent and when X2 is C, R2 is present;

X3 is C or N, and when X3 is N, R3 is absent; and
wherein when X3 is N, R3 is absent and when X3 is C, R3 is present;

X4 is C or N, and when X4 is N, R4 is absent;
wherein when X4 is N, R4 is absent and when X4 is C, R4 is present,
wherein
X2 is N and X3 and X4 are each C, or
X3 is N and X2 and X4 are each C, or
X3 and X4 are each N and X2 is C, or
X4 is N and X2 and X3 are each C, or
X2 is N, X3 is C and X4 are each N,

or a pharmaceutically acceptable salt thereof.

In some embodiments, the compound having the structure:

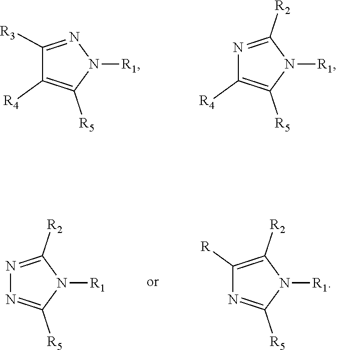

wherein

R1 is H, alkyl, alkenyl, alkynyl, cycloalkyl, alkylaryl, aryl, substituted aryl, heteroaryl or substituted heteroaryl; and

Each of R2, R3, R4 and R5 is present or absent, and when present is H, halogen, alkyl, alkenyl, alkynyl, cycloalkyl, cycloheteroalkyl, substituted cycloheteroalkyl, aryl, substituted aryl, heteroaryl, substituted heteroaryl, alkyl-N(alkyl)(CO)-lactam or alkyl-N(alkyl)(CO)-(aryl),

or a pharmaceutically acceptable salt thereof.

In some embodiments, the compound wherein one of R2, R3, R4 and R5 is other than H.

In some embodiments, the compound wherein two of R2, R3, R4 and R5 is other than H.

In some embodiments, the compound wherein R1 is H.

In some embodiments, the compound wherein R1 is other H.

In some embodiments, the compound having the structure:

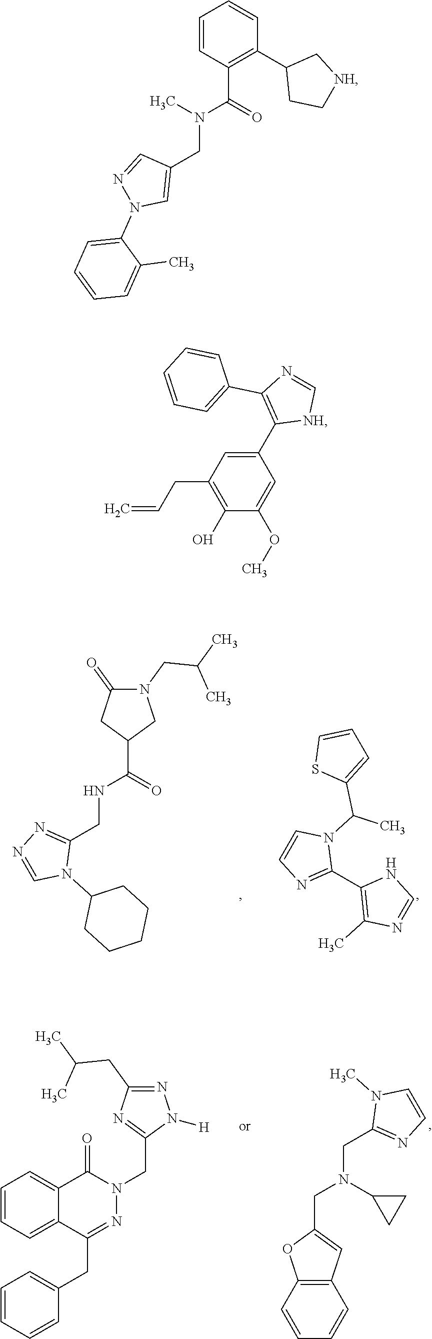

or a pharmaceutically acceptable salt thereof.

In some embodiments, the compound having the structure:

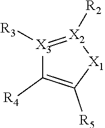

wherein

each of R2, R3, R4 and R5 is present or absent, and when present is H, halogen, alkyl, alkenyl, alkynyl, cycloalkyl, cycloheteroalkyl, substituted cycloheteroalkyl, aryl, substituted aryl, heteroaryl, substituted heteroaryl, R₆—N(R₇) (CO)-lactam, R₆—N(R₇)(CO)—(R₈), (CO)NH—R₇, R₆—NH-aryl, R₆—NH-heteroaryl, (CO)NH-(heteroaryl), R₆—N(R₇) (CO)-lactam, R₆(CO)-heterocycloalkyl, R₆(CO)NH—R₈, R₆(CO)NH-alkyl-R₈ or R₆(CO)NH-heteroalkyl-R₈;

X1 is O or S;

X2 is C or N,
wherein when X2 is N, R2 is absent and when X2 is C, R2 is present; and

X3 is C or N, and when X3 is N, R3 is absent;
wherein when X3 is N, R3 is absent and when X3 is C, R3 is present,
wherein
X1 is O and X2 and X3 are each C, or
X1 is S, X2 is C, and X3 are each N, or
X1 is O, X2 is N and X3 is C,
wherein each R6 is independently H, alkyl, alkenyl, alkynyl, alkylaryl, aryl or heteroaryl;
wherein each R7 and R8 is, independently, H, alkyl, alkenyl, alkynyl, arylalkyl, heteroarylalkyl, heterocycloalkylalkyl, aryl or heteroaryl, or R7 and R8 combine to form a cycloalkyl or heterocycloalkyl,

or a pharmaceutically acceptable salt thereof.

In some embodiments, the compound having the structure:

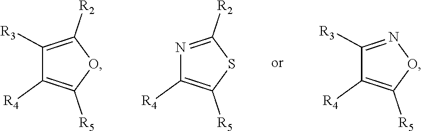

wherein

each of R2, R3, R4 and R5 is present or absent, and when present is H, halogen, alkyl, alkenyl, alkynyl, cycloalkyl, cycloheteroalkyl, substituted cycloheteroalkyl, aryl, substituted aryl, heteroaryl, substituted heteroaryl, R6-N(R7)(CO)-lactam, R6-N(R7)(CO)—(R8), (CO)NH—R₇, R₆—NH-aryl, R₆—NH-heteroaryl, (CO)NH-(heteroaryl), R₆—N(R₇)(CO)-lactam, R₆(CO)-heterocycloalkyl, R₆(CO)NH—R₈, R₆(CO)NH-alkyl-R₈ or R₆(CO)NH-heteroalkyl-R₈;

or a pharmaceutically acceptable salt thereof.

In some embodiments, the compound wherein one of R2, R3, R4 and R5 is other than H.

In some embodiments, the compound wherein two of R2, R3, R4 and R5 is other than H.

In some embodiments, the compound having of claim 1 having the structure:

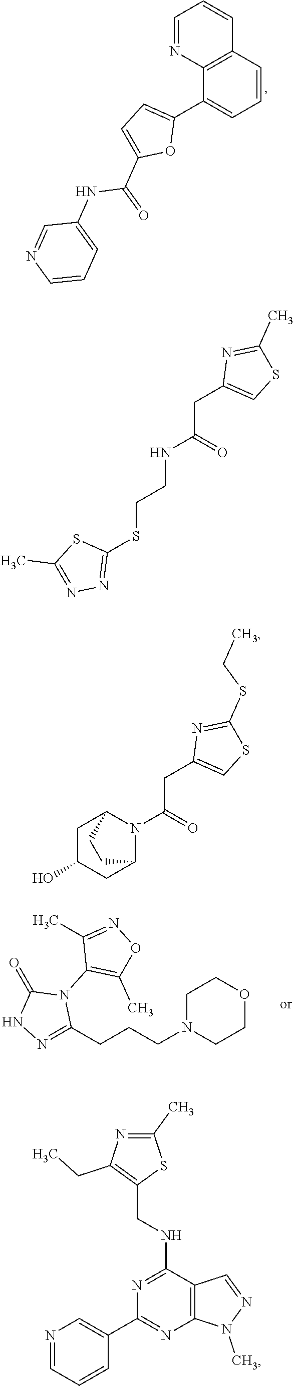

or a pharmaceutically acceptable salt thereof.

In some embodiments, the compound having the structure:

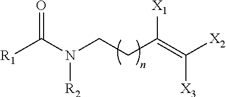

wherein

n is 0 or 1;

R1 is alkyl, alkenyl, alkynyl, cycloalkyl, cycloheteroalkyl, substituted cycloheteroalkyl, aryl, substituted aryl, heteroaryl, substituted heteroaryl, substituted heteroaryl, biaryl or substituted biaryl;

R2 is H, alkyl, alkyl, alkoxy, alkylamino or alkylaryl;

X1 is H, cycloalkyl, cycloheteroalkyl, substituted cycloheteroalkyl, aryl, substituted aryl, heteroaryl, substituted heteroaryl, substituted heteroaryl, biaryl or substituted biaryl;

X2 is H, cycloalkyl, cycloheteroalkyl, substituted cycloheteroalkyl, aryl, substituted aryl, heteroaryl, substituted heteroaryl, substituted heteroaryl, biaryl or substituted biaryl; and

X3 is H, cycloalkyl, cycloheteroalkyl, substituted cycloheteroalkyl, aryl, substituted aryl, heteroaryl, substituted heteroaryl, substituted heteroaryl, biaryl or substituted biaryl, or

X1 and X2 form a cycloalkenyl or cycloheteroalkenyl,

or a pharmaceutically acceptable salt thereof.

In some embodiments, the compound wherein

R1 is alkyl, aryl, substituted aryl, heteroaryl, substituted heteroaryl, biaryl or substituted biaryl.

In some embodiments, the compound wherein

R1 is an unsubstituted or substituted phenyl, pyridine, pyrimidine, pyrazine, indole, isoindole, furan, benzofuran, thiophene, benzothiophene, indazole, imidazole, benzimidazole, benzthiazole, quinoline, naphthyridine, isoquinoline or 4-phenyl-4H-triazole.

In some embodiments, the compound wherein

R1 is

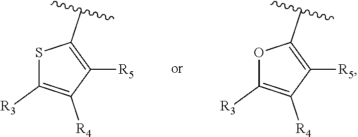

wherein

each of R3, R4 and R5 is, independently, H, halogen, OH, CF₃, OCF₃, OCHF₂, CN, NO₂, alkyl, haloalkyl, alkenyl, alkynyl, alkoxy, haloalkoxy, alkylamino, aryl, substituted aryl, heteroaryl or substituted heteroaryl.

In some embodiments, the compound wherein

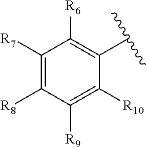

wherein

each of R6, R7, R8, R9 and R10 is, independently, H, halogen, OH, CF₃, OCF₃, OCHF₂, CN, NO₂, alkyl, haloalkyl, alkenyl, alkynyl, alkoxy, haloalkoxy, alkylamino, aryl, substituted aryl, heteroaryl or substituted heteroaryl.

In some embodiments, the compound wherein A has structure:

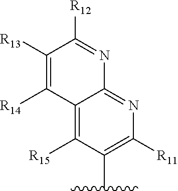

wherein

each of R11, R12, R13, R14 and R15 is, independently, H, halogen, OH, CF₃, OCF₃, OCHF₂, CN, NO₂, alkyl, haloalkyl, alkenyl, alkynyl, alkoxy, haloalkoxy, alkylamino, aryl, substituted aryl, heteroaryl or substituted heteroaryl.

In some embodiments, the compound wherein R1 is methyl or ethyl.

In some embodiments, the compound wherein R2 is H, methyl or ethyl.

In some embodiments, the compound wherein R2 is alkylaryl.

In some embodiments, the compound wherein

R2

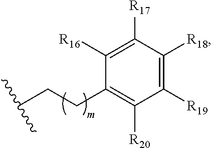

wherein

m is 0-5; and

each of R16, R17, R18, R19 and R20 is, independently, H, halogen, OH, CF₃, OCF₃, OCHF₂, CN, NO₂, alkyl, haloalkyl, alkenyl, alkynyl, alkoxy, haloalkoxy, alkylamino, aryl, substituted aryl, heteroaryl or substituted heteroaryl.

In some embodiments, the compound wherein

X1 is H; and

X2 is H and X3 is aryl, substituted aryl, heteroaryl or substituted heteroaryl, or X3 is H and X2 is aryl, substituted aryl, heteroaryl or substituted heteroaryl.

In some embodiments, the compound wherein

X1 and X2 form a cycloalkenyl or cycloheteroalkenyl; and

X3 is H.

In some embodiments, the compound having the structure:

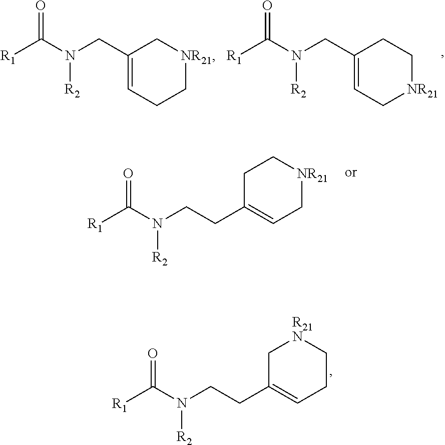

wherein R21 is H, alkyl, alkyl, haloalkyl, alkoxy, haloalkoxy or alkylamino.

In some embodiments, the compound wherein R21 is H, alkyl, alkyl, haloalkyl, alkyl-OH, alkyl-NH₂, alkyl-CF₂, or alkyl-aryl.

In some embodiments, the compound wherein R21 is alkyl-F, alkyl-Cl, alkyl-Br or alkyl-CF₃.

In some embodiments, the compound wherein R21

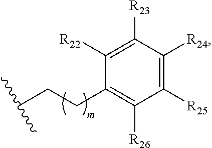

wherein

m is 0-5; and

each of R16, R17, R18, R19 and R20 is, independently, H, halogen, OH, CF₃, OCF₃, OCHF₂, CN, NO₂, alkyl, haloalkyl, alkenyl, alkynyl, alkoxy, haloalkoxy, alkylamino, aryl, substituted aryl, heteroaryl or substituted heteroaryl.

In some embodiments, the compound wherein R21

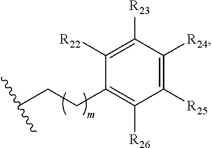

wherein

m is 1; and

each of R16, R17, R18, R19 and R20 is H or halogen.

In some embodiments, the compound having the structure:

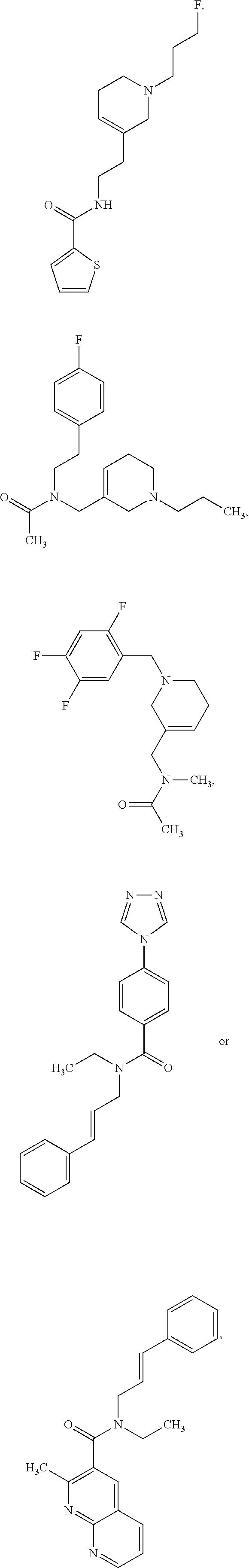 [SMILES: O=C(NCCC1=CCCN(CCCF)C1)c1cccs1 ; CCCN1CCC=C(CN(CCc2ccc(F)cc2)C(C)=O)C1 ; CC(=O)N(C)CC1=CCCN(Cc2cc(F)c(F)cc2F)C1 ; CCN(C/C=C/c1ccccc1)C(=O)c1ccc(-n2cnnc2)cc1 ; CCN(C/C=C/c1ccccc1)C(=O)c1cc2cccnc2nc1C]

or a pharmaceutically acceptable salt thereof.

In some embodiments, the compound having the structure:

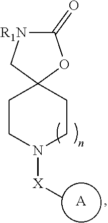

wherein

n is 0, 1 or 2;

R1 is H, alkyl, alkenyl, alkynyl, substituted cycloheteroalkyl, aryl, substituted aryl, heteroaryl or substituted heteroaryl;

X is present or absent and when present is a —(C=O)— or —(C═O)NH— linker; and

A is an aryl, substituted aryl, heteroaryl or substituted heteroaryl,

or a pharmaceutically acceptable salt thereof.

In some embodiments, the compound having the structure:

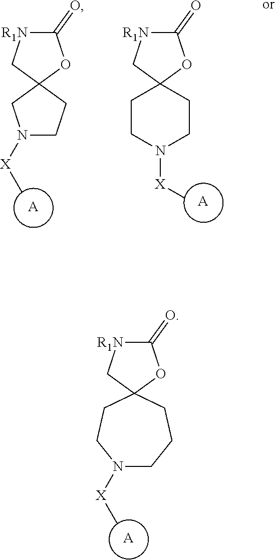

In some embodiments, the compound wherein R1 is H, methyl or ethyl.

In some embodiments, the compound wherein A is a substituted or unsubstituted lactam, phenyl, pyridine, pyrimidine, pyrazine, indole, isoindole, azaindole, benzofuran, benzothiophene, indazole, benzimidazole, benzthiazole, quinoline, naphthyridine, isoquinoline, dihydrobenzooxazine, tetrazole or pyrazolopyrimidine.

In some embodiments, the compound wherein A has the structure:

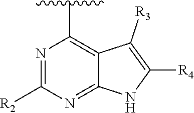

wherein

each of R2, R3 and R4 is, independently, H, halogen, OH, CF₃, OCF₃, OCHF₂, CN, NO₂, alkyl, haloalkyl, alkenyl, alkynyl, alkoxy, haloalkoxy, alkylamino, aryl, substituted aryl, heteroaryl or substituted heteroaryl.

In some embodiments, the compound wherein A has the structure:

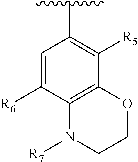

wherein

each of R5, R6 and R7 is, independently, H, halogen, OH, CF₃, OCF₃, OCHF₂, CN, NO₂, alkyl, haloalkyl, alkenyl, alkynyl, alkoxy, haloalkoxy, alkylamino, aryl, substituted aryl, heteroaryl or substituted heteroaryl.

In some embodiments, the compound wherein A has the structure:

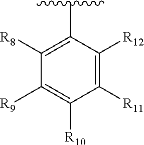

wherein

each of R8, R9, R10, R11 and R12 is, independently, H, halogen, OH, CF₃, OCF₃, OCHF₂, CN, NO₂, alkyl, haloalkyl, alkenyl, alkynyl, alkoxy, haloalkoxy, alkylamino, aryl, substituted aryl, heteroaryl or substituted heteroaryl.

In some embodiments, the compound wherein A has the structure:

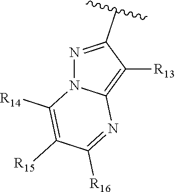

wherein

each of R8, R9, R10, R11 and R12 is, independently, H, halogen, OH, CF₃, OCF₃, OCHF₂, CN, NO₂, alkyl, haloalkyl, alkenyl, alkynyl, alkoxy, haloalkoxy, alkylamino, aryl, substituted aryl, heteroaryl or substituted heteroaryl.

In some embodiments, the compound wherein A has the structure:

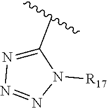

wherein

R17 is H, alkyl, haloalkyl, alkenyl, alkynyl, aryl, substituted aryl, heteroaryl or substituted heteroaryl.

In some embodiments, the compound wherein R1 is H, methyl or ethyl.

In some embodiments, the compound having the structure:

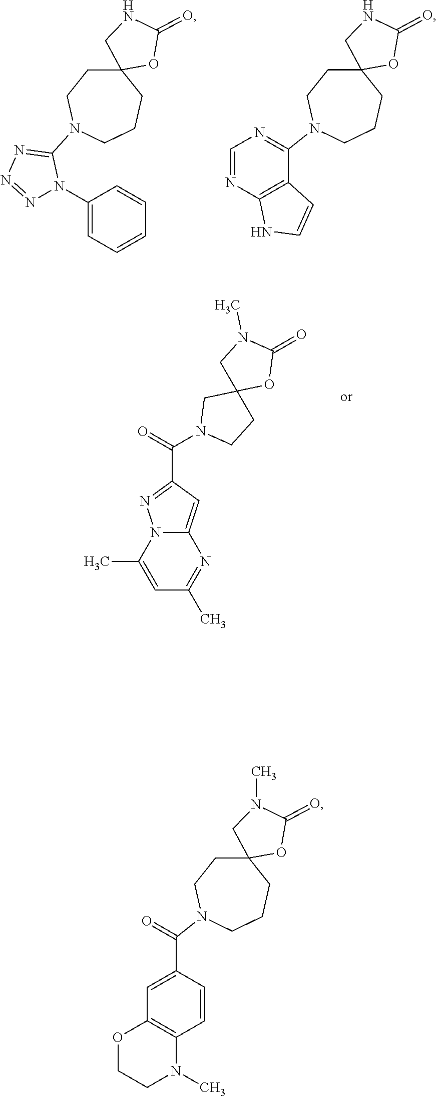 [SMILES: O=C1NCC2(CCCN(c3nnnn3-c3ccccc3)CC2)O1 ; O=C1NCC2(CCCN(c3ncnc4[nH]ccc34)CC2)O1 ; Cc1cc(C)n2nc(C(=O)N3CCC4(CN(C)C(=O)O4)C3)cc2n1 ; CN1CC2(CCCN(C(=O)c3ccc4c(c3)OCCN4C)CC2)OC1=O]

or a pharmaceutically acceptable salt thereof.

In some embodiments, the compound having the structure:

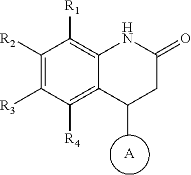

wherein

each of R1, R2, R3 and R4 is, independently, H, halogen, OH, CF₃, OCF₃, OCHF₂, CN, NO₂, alkyl, haloalkyl, alkenyl, alkynyl, alkoxy, haloalkoxy, alkylamino, aryl, substituted aryl, heteroaryl or substituted heteroaryl; and

A is an aryl, substituted aryl, biaryl, substituted biaryl, heteroaryl or substituted heteroaryl,

or a pharmaceutically acceptable salt thereof.

In some embodiments, the compound wherein A is a substituted or unsubstituted lactam, phenyl, pyridine, pyrimidine, pyrazine, thiophene, pyrazole, indole, isoindole, azaindole, benzofuran, benzothiophene, indazole, benzimidazole, benzthiazole, quinoline, naphthyridine, isoquinoline, dihydrobenzooxazine, tetrazole or pyrazolopyrimidine.

In some embodiments, the compound wherein A has the structure:

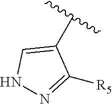

wherein

R5 is H, alkyl, haloalkyl, alkenyl, alkynyl, aryl, substituted aryl, heteroaryl or substituted heteroaryl.

In some embodiments, the compound wherein A has the structure:

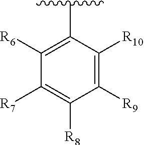

wherein

each of R6, R7, R8, R9 and R10 is, independently, H, halogen, OH, CF₃, OCF₃, OCHF₂, CN, NO₂, alkyl, haloalkyl, alkenyl, alkynyl alkoxy, haloalkoxy, alkylamino, aryl, substituted aryl, heteroaryl or substituted heteroaryl.

In some embodiments, the compound wherein A has the structure:

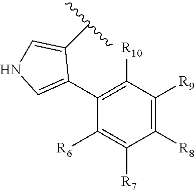

wherein

each of R6, R7, R8, R9 and R10 is, independently, H, halogen, OH, CF₃, OCF₃, OCHF₂, CN, NO₂, alkyl, haloalkyl, alkenyl, alkynyl alkoxy, haloalkoxy, alkylamino, aryl, substituted aryl, heteroaryl or substituted heteroaryl.

In some embodiments, the compound having the structure:

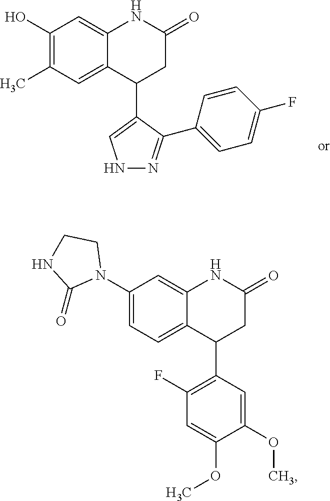 [SMILES: Cc1cc2c(cc1O)NC(=O)CC2c1c[nH]nc1-c1ccc(F)cc1 ; COc1cc(F)c(C2CC(=O)Nc3cc(N4CCNC4=O)ccc32)cc1OC]

or a pharmaceutically acceptable salt thereof.

In some embodiments, the compound having the structure:

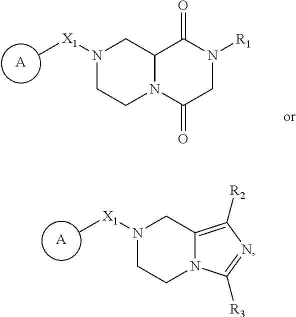

wherein

R1 is H, alkyl, haloalkyl, alkenyl, alkynyl, aryl, substituted aryl, heteroaryl or substituted heteroaryl;

each of R2 and R3 is, independently, H, halogen, OH, CF₃, OCF₃, OCHF₂, CN, NO₂, alkyl, haloalkyl, alkenyl, alkynyl, alkoxy, haloalkoxy, alkylamino, aryl, substituted aryl, heteroaryl or substituted heteroaryl; A is an aryl, substituted aryl, biaryl, substituted biaryl, heteroaryl or substituted heteroaryl; and

X1 is an alkyl, alkenyl, —(CO)— or —NH(CO)—,

or a pharmaceutically acceptable salt thereof.

In some embodiments, the compound wherein A is a substituted or unsubstituted lactam, phenyl, pyridine, pyrimidine, pyrazine, thiophene, pyrazole, indole, isoindole, azaindole, benzofuran, benzothiophene, indazole, benzimidazole, benzthiazole, quinoline, naphthyridine, isoquinoline, dihydrobenzooxazine, tetrazole or pyrazolopyrimidine.

In some embodiments, the compound wherein A has the structure:

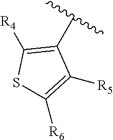

wherein

each of R4, R5 and R6 is, independently, H, halogen, OH, CF₃, OCF₃, OCHF₂, CN, NO₂, alkyl, haloalkyl, alkenyl, alkynyl, alkoxy, haloalkoxy, alkylamino, aryl, substituted aryl, heteroaryl or substituted heteroaryl.

In some embodiments, the compound wherein A has the structure:

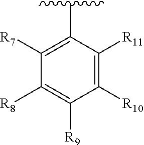

wherein

each of R7, R8, R9, R10 and R11 is, independently, H, halogen, OH, CF₃, OCF₃, OCHF₂, ON, NO₂, alkyl, haloalkyl, alkenyl, alkynyl, alkoxy, haloalkoxy, alkylamino, aryl, substituted aryl, heteroaryl or substituted heteroaryl.

In some embodiments, the compound wherein A has the structure:

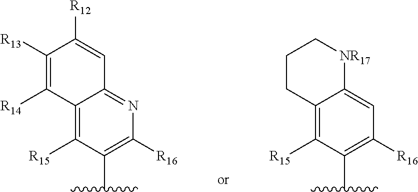

wherein

each of R12, R13, R14, R15 and R16 is, independently, H, halogen, OH, CF₃, OCF₃, OCHF₂, CN, NO₂, alkyl, haloalkyl, alkenyl, alkynyl, alkoxy, haloalkoxy, alkylamino, aryl, substituted aryl, heteroaryl or substituted heteroaryl; and

R17 is H, alkyl, haloalkyl, alkenyl, alkynyl, aryl, substituted aryl, heteroaryl or substituted heteroaryl.

In some embodiments, the compound wherein X1 is —(CH₂)—.

In some embodiments, the compound wherein X1 is —(CO)—.

In some embodiments, the compound having the structure:

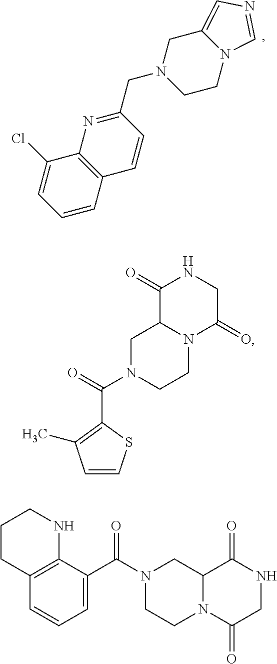 [SMILES: Clc1cccc2ccc(CN3CCn4cncc4C3)nc12 ; Cc1ccsc1C(=O)N1CCN2C(=O)CNC(=O)C2C1 ; O=C1NCC(=O)N2CCN(C(=O)c3cccc4c3NCCC4)CC12]

or a pharmaceutically acceptable salt thereof.

In some embodiments, the compound having the structure:

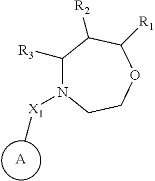

wherein

X1 is an alkyl, alkenyl, —(CO)— or —NH(CO)—;

R1 and R2 are each, independently, H, alkyl, haloalkyl, aminoalkyl, hydroxyalkyl, alkenyl, alkynyl, alkoxy, haloalkoxy, alkylamino, aryl, substituted aryl, biaryl, substituted biaryl, heteroaryl or substituted heteroaryl, or R1 and R2 combine to form a substituted or unsubstituted cycloalkyl, heterocycloalkyl, aryl or heteroaryl;

R3 is H, alkyl, haloalkyl, aminoalkyl, hydroxyalkyl, alkenyl, alkynyl, O-alkyl, O-haloalkyl, NH-alkyl, aryl, substituted aryl, biaryl, substituted biaryl, heteroaryl or substituted heteroaryl; and

A is an aryl, substituted aryl, biaryl, substituted biaryl, heteroaryl or substituted heteroaryl;

or a pharmaceutically acceptable salt thereof.

In some embodiments, the compound wherein A is a substituted or unsubstituted lactam, phenyl, pyridine, pyrimidine, pyrazine, thiophene, pyrazole, indole, isoindole, azaindole, benzofuran, benzothiophene, indazole, benzimidazole, benzthiazole, quinoline, naphthyridine, isoquinoline, dihydrobenzooxazine, tetrazole, pyrazolopyrimidine, imidazopyrimidine or tetrahydroimidazopyrazine.

In some embodiments, the compound wherein A is a substituted or unsubstituted imidazopyrimidine or tetrahydroimidazopyrazine.

In some embodiments, the wherein the compound has the structure:

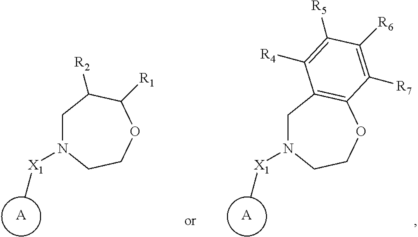

each of R4, R5, R6 and R7 is, independently, H, halogen, OH, CF₃, OCF₃, OCHF₂, CN, NO₂, alkyl, haloalkyl, alkenyl, alkynyl, alkoxy, haloalkoxy, alkylamino, aryl, substituted aryl, heteroaryl or substituted heteroaryl.

In some embodiments, the compound wherein A has the structure:

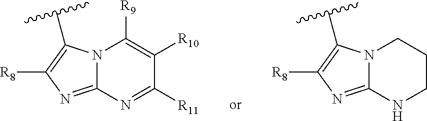

wherein

each of R8, R9, R10 and R11 is, independently, H, halogen, OH, CF₃, OCF₃, OCHF₂, CN, NO₂, alkyl, haloalkyl, alkenyl, alkynyl, alkoxy, haloalkoxy, alkylamino, aryl, substituted aryl, heteroaryl or substituted heteroaryl.

In some embodiments, the compound wherein A has the structure:

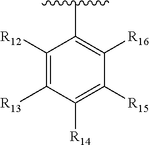

wherein

each of R12, R13, R14, R15 and R16 is, independently, H, halogen, OH, CF₃, OCF₃, OCHF₂, CN, NO₂, alkyl, haloalkyl, alkenyl, alkynyl, alkoxy, haloalkoxy, alkylamino, aryl, substituted aryl, heteroaryl or substituted heteroaryl.

In some embodiments, the compound wherein X1 is —(CH₂)—.

In some embodiments, the compound wherein X1 is —(CO)—.

In some embodiments, the compound having the structure:

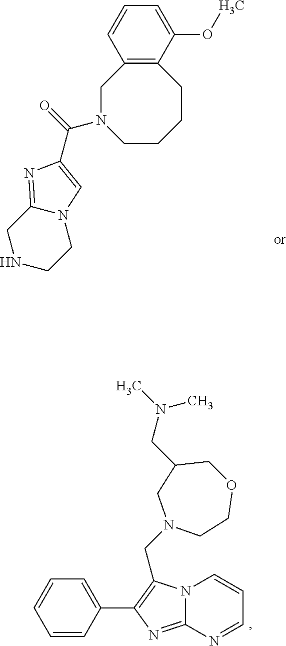 [SMILES: COc1cccc2c1CCCCN(C(=O)c1cn3c(n1)CNCC3)C2 ; CN(C)CC1COCCN(Cc2c(-c3ccccc3)nc3ncccn23)C1]

or a pharmaceutically acceptable salt thereof.

In some embodiments, the compound contains or is substituted with a fused bicyclic ring (i.e. indole, naphthyridine, quinolone, 3,4-dihydrobenzooxazine, 3,4-dihydroquinolinone or phthalazinone), which binds in a hydrophobic binding pocket of Cer1.

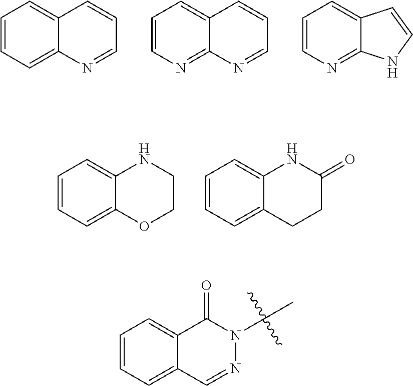

In any one of the embodiments of the above methods, inhibitors or compounds, the small molecule, inhibitor or compound has a structure other than any of the one or more structures recited in Table 4.

In some embodiments, the fungal ceramide synthase 1 (Cer1) is fungal ceramide synthase 1 (Cer1) 6717.

In some embodiments, the nucleotide sequence of the RNA, siRNA, shRNA, dsRNA, gRNA, or sgRNA molecule comprises or consists of a nucleotide sequence as set forth in any one of SEQ ID NOS: 1-8, or a nucleotide sequence complementary to the nucleotide sequence as set forth in any one of SEQ ID NOS: 1-8, or a nucleotide sequence lacking one or more nucleotides from the 5′ end of SEQ ID NOS: 1-8.

In some embodiments, the inhibitor of present invention targets a polypeptide or protein comprising or consisting of any one of SEQ ID NOS: 9-16.

SEQ ID NO. 1—Nucleotide sequence for Cer1

SEQ ID NO. 2—Nucleotide sequence for LAC1

SEQ ID NO. 3—Nucleotide sequence for “LAC1”, derived from BLAST, unable to find “LAC1” gene for Candida auris

SEQ ID NO. 4—Nucleotide sequence for LAC1, derived from BLAST

SEQ ID NO. 5—Nucleotide sequence for LAG1, derived from BLAST

SEQ ID NO. 6—Nucleotide sequence for LAC1

SEQ ID NO. 7—Nucleotide sequence for LAG1

SEQ ID NO. 8—Nucleotide sequence for LAC1

SEQ ID NO. 9—acyl-CoA-dependent ceramide synthase (Cer1) protein

SEQ ID NO. 10—longevity-assurance protein (LAC1)

SEQ ID NO. 11—“longevity-assurance protein (LAC1)” according to sequence listings attached in SUNY Mar. 18, 2018 email

SEQ ID NO. 12—longevity-assurance protein (LAC1)

SEQ ID NO. 13—sphingosine N-acyltransferase (lag1)

SEQ ID NO. 14—longevity-assurance protein (LAC1)

SEQ ID NO. 15—sphingosine N-acyltransferase (lag1)

SEQ ID NO. 16—longevity-assurance protein (LAC1)

The compounds of the present invention include all hydrates, solvates, and complexes of the compounds used by this invention. If a chiral center or another form of an isomeric center is present in a compound of the present invention, all forms of such isomer or isomers, including enantiomers and diastereomers, are intended to be covered herein. Compounds containing a chiral center may be used as a racemic mixture, an enantiomerically enriched mixture, or the racemic mixture may be separated using well-known techniques and an individual enantiomer may be used alone. The compounds described in the present invention are in racemic form or as individual enantiomers. The enantiomers can be separated using known techniques, such as those described in Pure and Applied Chemistry 69, 1469-1474, (1997) IUPAC. In cases in which compounds have unsaturated carbon-carbon double bonds, both the cis (Z) and trans (E) isomers are within the scope of this invention.

The compounds of the subject invention may have spontaneous tautomeric forms. In cases wherein compounds may exist in tautomeric forms, such as keto-enol tautomers, each tautomeric form is contemplated as being included within this invention whether existing in equilibrium or predominantly in one form.

In the compound structures depicted herein, hydrogen atoms are not shown for carbon atoms having less than four bonds to non-hydrogen atoms. However, it is understood that enough hydrogen atoms exist on said carbon atoms to satisfy the octet rule.

This invention also provides isotopic variants of the compounds disclosed herein, including wherein the isotopic atom is ²H and/or wherein the isotopic atom ¹³C. Accordingly, in the compounds provided herein hydrogen can be enriched in the deuterium isotope. It is to be understood that the invention encompasses all such isotopic forms.

It is understood that the structures described in the embodiments of the methods hereinabove can be the same as the structures of the compounds described hereinabove.

It is understood that where a numerical range is recited herein, the present invention contemplates each integer between, and including, the upper and lower limits, unless otherwise stated.

Except where otherwise specified, if the structure of a compound of this invention includes an asymmetric carbon atom, it is understood that the compound occurs as a racemate, racemic mixture, and isolated single enantiomer. All such isomeric forms of these compounds are expressly included in this invention. Except where otherwise specified, each stereogenic carbon may be of the R or S configuration. It is to be understood accordingly that the isomers arising from such asymmetry (e.g., all enantiomers and diastereomers) are included within the scope of this invention, unless indicated otherwise. Such isomers can be obtained in substantially pure form by classical separation techniques and by stereochemically controlled synthesis, such as those described in “Enantiomers, Racemates and Resolutions” by J. Jacques, A. Collet and S. Wilen, Pub. John Wiley & Sons, N Y, 1981. For example, the resolution may be carried out by preparative chromatography on a chiral column.

The subject invention is also intended to include all isotopes of atoms occurring on the compounds disclosed herein. Isotopes include those atoms having the same atomic number but different mass numbers. By way of general example and without limitation, isotopes of hydrogen include tritium and deuterium. Isotopes of carbon include C-13 and C-14.

It will be noted that any notation of a carbon in structures throughout this application, when used without further notation, are intended to represent all isotopes of carbon, such as ¹²C, ¹³C, or ¹⁴C. Furthermore, any compounds containing ¹³C or ¹⁴C may specifically have the structure of any of the compounds disclosed herein.

It will also be noted that any notation of a hydrogen in structures throughout this application, when used without further notation, are intended to represent all isotopes of hydrogen, such as ¹H, ²H, or ³H. Furthermore, any compounds containing ²H or ³H may specifically have the structure of any of the compounds disclosed herein.

Isotopically-labeled compounds can generally be prepared by conventional techniques known to those skilled in the art using appropriate isotopically-labeled reagents in place of the non-labeled reagents employed.

In the compounds used in the method of the present invention, the substituents may be substituted or unsubstituted, unless specifically defined otherwise.

In the compounds used in the method of the present invention, alkyl, heteroalkyl, monocycle, bicycle, aryl, heteroaryl and heterocycle groups can be further substituted by replacing one or more hydrogen atoms with alternative non-hydrogen groups. These include, but are not limited to, halo, hydroxy, mercapto, amino, carboxy, cyano, carbamoyl and aminocarbonyl and aminothiocarbonyl.

It is understood that substituents and substitution patterns on the compounds used in the method of the present invention can be selected by one of ordinary skill in the art to provide compounds that are chemically stable and that can be readily synthesized by techniques known in the art from readily available starting materials. If a substituent is itself substituted with more than one group, it is understood that these multiple groups may be on the same carbon or on different carbons, so long as a stable structure results.

In choosing the compounds used in the method of the present invention, one of ordinary skill in the art will recognize that the various substituents, i.e. R₁, R₂, etc. are to be chosen in conformity with well-known principles of chemical structure connectivity.

As used herein, “alkyl” is intended to include both branched and straight-chain saturated aliphatic hydrocarbon groups having the specified number of carbon atoms. Thus, C₁-Cₙ as in “C₁-Cₙalkyl” is defined to include groups having 1, 2, . . . , n−1 or n carbons in a linear or branched arrangement, and specifically includes methyl, ethyl, propyl, butyl, pentyl, hexyl, heptyl, isopropyl, isobutyl, sec-butyl and so on. An embodiment can be C₁-C₁₂ alkyl, C₂-C₁₂ alkyl, C₃-C₁₂ alkyl, C₄-C₁₂ alkyl and so on. “Alkoxy” represents an alkyl group as described above attached through an oxygen bridge.

The term “alkenyl” refers to a non-aromatic hydrocarbon radical, straight or branched, containing at least 1 carbon to carbon double bond, and up to the maximum possible number of non-aromatic carbon-carbon double bonds may be present. Thus, C₂-Cₙ alkenyl is defined to include groups having 1, 2 . . . , n−1 or n carbons. For example, “C₂-C₆ alkenyl” means an alkenyl radical having 2, 3, 4, 5, or 6 carbon atoms, and at least 1 carbon-carbon double bond, and up to, for example, 3 carbon-carbon double bonds in the case of a C₆ alkenyl, respectively. Alkenyl groups include ethenyl, propenyl, butenyl and cyclohexenyl. As described above with respect to alkyl, the straight, branched or cyclic portion of the alkenyl group may contain double bonds and may be substituted if a substituted alkenyl group is indicated. An embodiment can be C₂-C₁₂ alkenyl, C₃-C₁₂ alkenyl, C₄-C₁₂ alkenyl and so on.

The term “alkynyl” refers to a hydrocarbon radical straight or branched, containing at least 1 carbon to carbon triple bond, and up to the maximum possible number of non-aromatic carbon-carbon triple bonds may be present. Thus, C₂-Cₙ alkynyl is defined to include groups having 1, 2 . . . , n−1 or n carbons. For example, “C₂-C₆ alkynyl” means an alkynyl radical having 2 or 3 carbon atoms, and 1 carbon-carbon triple bond, or having 4 or 5 carbon atoms, and up to 2 carbon-carbon triple bonds, or having 6 carbon atoms, and up to 3 carbon-carbon triple bonds. Alkynyl groups include ethynyl, propynyl and butynyl. As described above with respect to alkyl, the straight or branched portion of the alkynyl group may contain triple bonds and may be substituted if a substituted alkynyl group is indicated. An embodiment can be a C₂-Cₙalkynyl. An embodiment can be C₂-C₁₂ alkynyl, C₃-C₁₂ alkynyl, C₄-C₁₂ alkynyl and so on

“Alkylene”, “alkenylene” and “alkynylene” shall mean, respectively, a divalent alkane, alkene and alkyne radical, respectively. It is understood that an alkylene, alkenylene, and alkynylene may be straight or branched. An alkylene, alkenylene, and alkynylene may be unsubstituted or substituted.

As used herein, “heteroalkyl” includes both branched and straight-chain saturated aliphatic hydrocarbon groups having the specified number of carbon atoms and at least 1 heteroatom within the chain or branch.

As used herein, “heterocycle” or “heterocyclyl” as used herein is intended to mean a 5- to 10-membered nonaromatic ring containing from 1 to 4 heteroatoms selected from the group consisting of O, N and S, and includes bicyclic groups. “Heterocyclyl” therefore includes, but is not limited to the following: imidazolyl, piperazinyl, piperidinyl, pyrrolidinyl, morpholinyl, thiomorpholinyl, tetrahydropyranyl, dihydropiperidinyl, tetrahydrothiophenyl and the like. If the heterocycle contains a nitrogen, it is understood that the corresponding N-oxides thereof are also encompassed by this definition.

As herein, “cycloalkyl” shall mean cyclic rings of alkanes of three to eight total carbon atoms, or any number within this range (i.e., cyclopropyl, cyclobutyl, cyclopentyl, cyclohexyl, cycloheptyl or cyclooctyl).

As used herein, “monocycle” includes any stable polyatomic carbon ring of up to 10 atoms and may be unsubstituted or substituted. Examples of such non-aromatic monocycle elements include but are not limited to: cyclobutyl, cyclopentyl, cyclohexyl, and cycloheptyl. Examples of such aromatic monocycle elements include but are not limited to: phenyl.

As used herein, “bicycle” includes any stable polyatomic carbon ring of up to 10 atoms that is fused to a polyatomic carbon ring of up to 10 atoms with each ring being independently unsubstituted or substituted. Examples of such non-aromatic bicycle elements include but are not limited to: decahydronaphthalene. Examples of such aromatic bicycle elements include but are not limited to: naphthalene.

As used herein, “aryl” is intended to mean any stable monocyclic, bicyclic or polycyclic carbon ring of up to 10 atoms in each ring, wherein at least one ring is aromatic, and may be unsubstituted or substituted. Examples of such aryl elements include phenyl, p-toluenyl (4-methylphenyl), naphthyl, tetrahydro-naphthyl, indanyl, biphenyl, phenanthryl, anthryl or acenaphthyl. In cases where the aryl substituent is bicyclic and one ring is non-aromatic, it is understood that attachment is via the aromatic ring.

As used herein, the term “polycyclic” refers to unsaturated or partially unsaturated multiple fused ring structures, which may be unsubstituted or substituted.

The term “arylalkyl” refers to alkyl groups as described above wherein one or more bonds to hydrogen contained therein are replaced by a bond to an aryl group as described above. It is understood that an “arylalkyl” group is connected to a core molecule through a bond from the alkyl group and that the aryl group acts as a substituent on the alkyl group. Examples of arylalkyl moieties include, but are not limited to, benzyl (phenylmethyl), p-trifluoromethylbenzyl (4-trifluoromethylphenylmethyl), 1-phenylethyl, 2-phenylethyl, 3-phenylpropyl, 2-phenylpropyl and the like.

The term “heteroaryl”, as used herein, represents a stable monocyclic, bicyclic or polycyclic ring of up to 10 atoms in each ring, wherein at least one ring is aromatic and contains from 1 to 4 heteroatoms selected from the group consisting of O, N and S. Bicyclic aromatic heteroaryl groups include phenyl, pyridine, pyrimidine or pyridizine rings that are (a) fused to a 6-membered aromatic (unsaturated) heterocyclic ring having one nitrogen atom; (b) fused to a 5- or 6-membered aromatic (unsaturated) heterocyclic ring having two nitrogen atoms; (c) fused to a 5-membered aromatic (unsaturated) heterocyclic ring having one nitrogen atom together with either one oxygen or one sulfur atom; or (d) fused to a 5-membered aromatic (unsaturated) heterocyclic ring having one heteroatom selected from O, N or S. Heteroaryl groups within the scope of this definition include but are not limited to: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrazolyl, tetrazolopyridyl, thiadiazolyl, thiazolyl, thienyl, triazolyl, azetidinyl, aziridinyl, 1,4-dioxanyl, hexahydroazepinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, tetrahydrothienyl, acridinyl, carbazolyl, cinnolinyl, quinoxalinyl, pyrrazolyl, indolyl, benzotriazolyl, benzothiazolyl, benzoxazolyl, isoxazolyl, isothiazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetra-hydroquinoline. In cases where the heteroaryl substituent is bicyclic and one ring is non-aromatic or contains no heteroatoms, it is understood that attachment is via the aromatic ring or via the heteroatom containing ring, respectively. If the heteroaryl contains nitrogen atoms, it is understood that the corresponding N-oxides thereof are also encompassed by this definition.

The term “heteroarylalkyl” refers to alkyl groups as described above wherein one or more bonds to hydrogen contained therein are replaced by a bond to an heteroaryl group as described above. It is understood that an “heteroarylalkyl” group is connected to a core molecule through a bond from the alkyl group and that the heteroaryl group acts as a substituent on the alkyl group. Examples of heteroarylalkyl moieties include, but are not limited to, —CH₂—(C₅H₄N), —CH₂—CH₂—(C₅H₄N) and the like.

The term “heterocycle” or “heterocyclyl” refers to a mono- or poly-cyclic ring system which can be saturated or contains one or more degrees of unsaturation and contains one or more heteroatoms. Preferred heteroatoms include N, O, and/or S, including N-oxides, sulfur oxides, and dioxides. Preferably the ring is three to ten-membered and is either saturated or has one or more degrees of unsaturation. The heterocycle may be unsubstituted or substituted, with multiple degrees of substitution being allowed. Such rings may be optionally fused to one or more of another “heterocyclic” ring(s), heteroaryl ring(s), aryl ring(s), or cycloalkyl ring(s). Examples of heterocycles include, but are not limited to, tetrahydrofuran, pyran, 1,4-dioxane, 1,3-dioxane, piperidine, piperazine, pyrrolidine, morpholine, thiomorpholine, tetrahydrothiopyran, tetrahydrothiophene, 1,3-oxathiolane, and the like.

The alkyl, alkenyl, alkynyl, aryl, heteroaryl and heterocyclyl substituents may be substituted or unsubstituted, unless specifically defined otherwise. In the compounds of the present invention, alkyl, alkenyl, alkynyl, aryl, heterocyclyl and heteroaryl groups can be further substituted by replacing one or more hydrogen atoms with alternative non-hydrogen groups. These include, but are not limited to, halo, hydroxy, mercapto, amino, carboxy, cyano and carbamoyl.

As used herein, the term “halogen” refers to F, Cl, Br, and I.

The terms “substitution”, “substituted” and “substituent” refer to a functional group as described above in which one or more bonds to a hydrogen atom contained therein are replaced by a bond to non-hydrogen or non-carbon atoms, provided that normal valencies are maintained and that the substitution results in a stable compound. Substituted groups also include groups in which one or more bonds to a carbon(s) or hydrogen(s) atom are replaced by one or more bonds, including double or triple bonds, to a heteroatom. Examples of substituent groups include the functional groups described above, and halogens (i.e., F, Cl, Br, and I); alkyl groups, such as methyl, ethyl, n-propyl, isopropryl, n-butyl, tert-butyl, and trifluoromethyl; hydroxyl; alkoxy groups, such as methoxy, ethoxy, n-propoxy, and isopropoxy; aryloxy groups, such as phenoxy; arylalkyloxy, such as benzyloxy (phenylmethoxy) and p-trifluoromethylbenzyloxy (4-trifluoromethylphenylmethoxy); heteroaryloxy groups; sulfonyl groups, such as trifluoromethanesulfonyl, methanesulfonyl, and p-toluenesulfonyl; nitro, nitrosyl; mercapto; sulfanyl groups, such as methylsulfanyl, ethylsulfanyl and propylsulfanyl; cyano; amino groups, such as amino, methylamino, dimethylamino, ethylamino, and diethylamino; and carboxyl. Where multiple substituent moieties are disclosed or claimed, the substituted compound can be independently substituted by one or more of the disclosed or claimed substituent moieties, singly or pluraly. By independently substituted, it is meant that the (two or more) substituents can be the same or different. Further examples of substituent groups include halogen, alkly, alkoxy, triazole, pyrrolidino, morpholino, triazolone, lactam or imidazolidinone.

Alkoxy, in a non-limiting example, may be O-alkyl. Haloalkyl, in a non-limiting example, may be alkyl-halogen. Aryloxy, in a non-limiting example, may be O-aryl. Alkylamino, in a non-limiting example, may be alkly-NH₂.

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

In choosing the compounds of the present invention, one of ordinary skill in the art will recognize that the various substituents, i.e. R₁, R₂, etc. are to be chosen in conformity with well-known principles of chemical structure connectivity.

The various R groups attached to the aromatic rings of the compounds disclosed herein may be added to the rings by standard procedures, for example those set forth in Advanced Organic Chemistry: Part B: Reaction and Synthesis, Francis Carey and Richard Sundberg, (Springer) 5th ed. Edition. (2007), the content of which is hereby incorporated by reference.

The compounds used in the method of the present invention may be prepared by techniques well known in organic synthesis and familiar to a practitioner ordinarily skilled in the art. However, these may not be the only means by which to synthesize or obtain the desired compounds.

The compounds used in the method of the present invention may be prepared by techniques described in Vogel's Textbook of Practical Organic Chemistry, A. I. Vogel, A. R. Tatchell, B. S. Furnis, A. J. Hannaford, P. W. G. Smith, (Prentice Hall) 5th Edition (1996), March's Advanced Organic Chemistry: Reactions, Mechanisms, and Structure, Michael B. Smith, Jerry March, (Wiley-Interscience) 5^{th} Edition (2007), and references therein, which are incorporated by reference herein. However, these may not be the only means by which to synthesize or obtain the desired compounds. Another aspect of the invention comprises a compound used in the method of the present invention as a pharmaceutical composition.

In some embodiments, a pharmaceutical composition comprises the compound of the present invention and a pharmaceutically acceptable carrier.

As used herein, the term “pharmaceutically active agent” means any substance or compound suitable for administration to a subject and furnishes biological activity or other direct effect in the treatment, cure, mitigation, diagnosis, or prevention of disease, or affects the structure or any function of the subject. Pharmaceutically active agents include, but are not limited to, substances and compounds described in the Physicians' Desk Reference (PDR Network, LLC; 64th edition; Nov. 15, 2009) and “Approved Drug Products with Therapeutic Equivalence Evaluations” (U.S. Department Of Health And Human Services, 30th edition, 2010), which are hereby incorporated by reference. Pharmaceutically active agents which have pendant carboxylic acid groups may be modified in accordance with the present invention using standard esterification reactions and methods readily available and known to those having ordinary skill in the art of chemical synthesis. Where a pharmaceutically active agent does not possess a carboxylic acid group, the ordinarily skilled artisan will be able to design and incorporate a carboxylic acid group into the pharmaceutically active agent where esterification may subsequently be carried out so long as the modification does not interfere with the pharmaceutically active agent's biological activity or effect.

The compounds used in the method of the present invention may be in a salt form. As used herein, a “salt” is a salt of the instant compounds which has been modified by making acid or base salts of the compounds. In the case of compounds used to treat an infection or disease caused by a pathogen, the salt is pharmaceutically acceptable. Examples of pharmaceutically acceptable salts include, but are not limited to, mineral or organic acid salts of basic residues such as amines; alkali or organic salts of acidic residues such as phenols. The salts can be made using an organic or inorganic acid. Such acid salts are chlorides, bromides, sulfates, nitrates, phosphates, sulfonates, formates, tartrates, maleates, malates, citrates, benzoates, salicylates, ascorbates, and the like. Phenolate salts are the alkaline earth metal salts, sodium, potassium or lithium. The term “pharmaceutically acceptable salt” in this respect, refers to the relatively non-toxic, inorganic and organic acid or base addition salts of compounds of the present invention. These salts can be prepared in situ during the final isolation and purification of the compounds of the invention, or by separately reacting a purified compound of the invention in its free base or free acid form with a suitable organic or inorganic acid or base, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci. 66:1-19).

The compounds of the present invention may also form salts with basic amino acids such a lysine, arginine, etc. and with basic sugars such as N-methylglucamine, 2-amino-2-deoxyglucose, etc. and any other physiologically non-toxic basic substance.

As used herein, “administering” an agent may be performed using any of the various methods or delivery systems well known to those skilled in the art. The administering can be performed, for example, orally, parenterally, intraperitoneally, intravenously, intraarterially, transdermally, sublingually, intramuscularly, rectally, transbuccally, intranasally, liposomally, via inhalation, vaginally, intraoccularly, via local delivery, subcutaneously, intraadiposally, intraarticularly, intrathecally, into a cerebral ventricle, intraventicularly, intratumorally, into cerebral parenchyma or intraparenchchymally.

The compounds used in the method of the present invention may be administered in various forms, including those detailed herein. The treatment with the compound may be a component of a combination therapy or an adjunct therapy, i.e. the subject or patient in need of the drug is treated or given another drug for the disease in conjunction with one or more of the instant compounds. This combination therapy can be sequential therapy where the patient is treated first with one drug and then the other or the two drugs are given simultaneously. These can be administered independently by the same route or by two or more different routes of administration depending on the dosage forms employed.

As used herein, a “pharmaceutically acceptable carrier” is a pharmaceutically acceptable solvent, suspending agent or vehicle, for delivering the instant compounds to the animal or human. The carrier may be liquid or solid and is selected with the planned manner of administration in mind. Liposomes are also a pharmaceutically acceptable carrier as are slow-release vehicles.

The dosage of the compounds administered in treatment will vary depending upon factors such as the pharmacodynamic characteristics of a specific chemotherapeutic agent and its mode and route of administration; the age, sex, metabolic rate, absorptive efficiency, health and weight of the recipient; the nature and extent of the symptoms; the kind of concurrent treatment being administered; the frequency of treatment with; and the desired therapeutic effect.

A dosage unit of the compounds used in the method of the present invention may comprise a single compound or mixtures thereof with additional antitumor agents. The compounds can be administered in oral dosage forms as tablets, capsules, pills, powders, granules, elixirs, tinctures, suspensions, syrups, and emulsions. The compounds may also be administered in intravenous (bolus or infusion), intraperitoneal, subcutaneous, or intramuscular form, or introduced directly, e.g. by injection, topical application, or other methods, into or topically onto a site of disease or lesion, all using dosage forms well known to those of ordinary skill in the pharmaceutical arts.

The compounds used in the method of the present invention can be administered in admixture with suitable pharmaceutical diluents, extenders, excipients, or in carriers such as the novel programmable sustained-release multi-compartmental nanospheres (collectively referred to herein as a pharmaceutically acceptable carrier) suitably selected with respect to the intended form of administration and as consistent with conventional pharmaceutical practices. The unit will be in a form suitable for oral, nasal, rectal, topical, intravenous or direct injection or parenteral administration. The compounds can be administered alone or mixed with a pharmaceutically acceptable carrier. This carrier can be a solid or liquid, and the type of carrier is generally chosen based on the type of administration being used. The active agent can be co-administered in the form of a tablet or capsule, liposome, as an agglomerated powder or in a liquid form. Examples of suitable solid carriers include lactose, sucrose, gelatin and agar. Capsule or tablets can be easily formulated and can be made easy to swallow or chew; other solid forms include granules, and bulk powders. Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms may also include minerals and other materials to make them compatible with the type of injection or delivery system chosen.

Techniques and compositions for making dosage forms useful in the present Invention are described in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol. 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modem Pharmaceutics Drugs and the Pharmaceutical Sciences, Vol 40 (Gilbert S. Banker, Christopher T. Rhodes, Eds.). All of the aforementioned publications are incorporated by reference herein.

Tablets may contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol and the like. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

The compounds used in the method of the present invention may also be administered in the form of liposome delivery systems, such as small unilamellar vesicles, large unilamellar vesicles, and multilamellar vesicles. Liposomes can be formed from a variety of phospholipids such as lecithin, sphingomyelin, proteolipids, protein-encapsulated vesicles or from cholesterol, stearylamine, or phosphatidylcholines. The compounds may be administered as components of tissue-targeted emulsions.

The compounds used in the method of the present invention may also be coupled to soluble polymers as targetable drug carriers or as a prodrug. Such polymers include polyvinylpyrrolidone, pyran copolymer, polyhydroxylpropylmethacrylamide-phenol, polyhydroxyethylasparta-midephenol, or polyethyleneoxide-polylysine substituted with palmitoyl residues. Furthermore, the compounds may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Gelatin capsules may contain the active ingredient compounds and powdered carriers, such as lactose, starch, cellulose derivatives, magnesium stearate, stearic acid, and the like. Similar diluents can be used to make compressed tablets. Both tablets and capsules can be manufactured as immediate release products or as sustained release products to provide for continuous release of medication over a period of hours. Compressed tablets can be sugar-coated or film-coated to mask any unpleasant taste and protect the tablet from the atmosphere, or enteric coated for selective disintegration in the gastrointestinal tract.

For oral administration in liquid dosage form, the oral drug components are combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents.

Liquid dosage forms for oral administration can contain coloring and flavoring to increase patient acceptance. In general, water, asuitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. Suitable pharmaceutical carriers are described in Remington's Pharmaceutical Sciences, Mack Publishing Company, a standard reference text in this field.

The compounds used in the method of the present invention may also be administered in intranasal form via use of suitable intranasal vehicles, or via transdermal routes, using those forms of transdermal skin patches well known to those of ordinary skill in that art. To be administered in the form of a transdermal delivery system, the dosage administration will generally be continuous rather than intermittent throughout the dosage regimen.

Parenteral and intravenous forms may also include minerals and other materials such as solutol and/or ethanol to make them compatible with the type of injection or delivery system chosen.

The compounds and compositions of the present invention can be administered in oral dosage forms as tablets, capsules, pills, powders, granules, elixirs, tinctures, suspensions, syrups, and emulsions. The compounds may also be administered in intravenous (bolus or infusion), intraperitoneal, subcutaneous, or intramuscular form, or introduced directly, e.g. by topical administration, injection or other methods, to the afflicted area, such as a wound, including ulcers of the skin, all using dosage forms well known to those of ordinary skill in the pharmaceutical arts.

Specific examples of pharmaceutically acceptable carriers and excipients that may be used to formulate oral dosage forms of the present invention are described in U.S. Pat. No. 3,903,297 to Robert, issued Sep. 2, 1975. Techniques and compositions for making dosage forms useful in the present invention are described-in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modem Pharmaceutics Drugs and the Pharmaceutical Sciences, Vol 40 (Gilbert S. Banker, Christopher T. Rhodes, Eds.). All of the aforementioned publications are incorporated by reference herein.

The active ingredient can be administered orally in solid dosage forms, such as capsules, tablets, powders, and chewing gum; or in liquid dosage forms, such as elixirs, syrups, and suspensions, including, but not limited to, mouthwash and toothpaste. It can also be administered parentally, in sterile liquid dosage forms.

Solid dosage forms, such as capsules and tablets, may be enteric-coated to prevent release of the active ingredient compounds before they reach the small intestine. Materials that may be used as enteric coatings include, but are not limited to, sugars, fatty acids, proteinaceous substances such as gelatin, waxes, shellac, cellulose acetate phthalate (CAP), methyl acrylate-methacrylic acid copolymers, cellulose acetate succinate, hydroxy propyl methyl cellulose phthalate, hydroxy propyl methyl cellulose acetate succinate (hypromellose acetate succinate), polyvinyl acetate phthalate (PVAP), and methyl methacrylate-methacrylic acid copolymers.

The compounds and compositions of the invention can be coated onto stents for temporary or permanent implantation into the cardiovascular system of a subject.

Variations on those general synthetic methods will be readily apparent to those of ordinary skill in the art and are deemed to be within the scope of the present invention.

Each embodiment disclosed herein is contemplated as being applicable to each of the other disclosed embodiments. Thus, all combinations of the various elements described herein are within the scope of the invention.

This invention will be better understood by reference to the Experimental Details which follow, but those skilled in the art will readily appreciate that the specific experiments detailed are only illustrative of the invention as described more fully in the claims which follow thereafter.

Methods of Gene Expression Editing

This invention also provides methods for editing the gene expression for fungal ceramide synthase. There is provided, in accordance with an embodiment, a method for knocking out a gene. In some embodiments, a gene is deactivated by delivering to a cell a guide RNA which targets a SNP in the promoter region, the start codon, or the untranslated region (UTR) of the gene.

“Nucleic acid molecule” refers to a polynucleotide such as, for example, DNA, RNA or oligonucleotides. It may be DNA or RNA of genomic or synthetic origin, double-stranded or single-stranded.

A “gene,” for the purposes of the present disclosure, includes a DNA region encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

For each gene, according to SNP/insertion/deletion/indel identified based on a selection criteria, any one of the following strategies may be used to deactivate the gene: (1) Knockout strategy using one guide RNA—one guide RNA is utilized to direct a CRISPR nuclease to a gene and create a double-strand break (DSB) leading to formation of a frameshift mutation in an exon or in a splice site region of the gene; (2) Knockout strategy using two guide RNAs—two guide RNAs are utilized. A first guide RNA targets a region in the promoter or an upstream region of a gene and a second guide RNA targets downstream of the first guide RNA in a promoter, exon, or intron of the gene; (3) Exon(s) skipping strategy—one guide RNA may be used to target a CRISPR nuclease to a splice site region, either at the 5′end of an intron (donor sequence) or the 3′ end of an intron (acceptor sequence), in order to destroy the splice site. Alternatively, two guide RNAs may be utilized such that a first guide RNA targets an upstream region of an exon and a second guide RNA targets a region downstream of the first guide RNA, thereby excising the exon(s). Based on the locations of identified SNPs/insertions/deletions/indels for each mutant allele, any one of, or a combination of, the above-mentioned methods to deactivate the mutant allele may be utilized.

The term guide RNA (gRNA) refers to an RNA molecule capable of targeting a CRISPR nuclease to a specific DNA sequence e.g., a genomic DNA sequence having a nucleotide sequence which is complementary to said gRNA. A guide RNA can be custom designed to target any desired sequence.

The term “single guide RNA” (sgRNA), is an RNA molecule that can form a complex with a CRISPR nuclease e.g., Cas9 and serve as the DNA targeting module. sgRNA is designed as a synthetic fusion of the CRISPR RNA (crRNA, or guide RNA) and the trans-activating crRNA (tracrRNA). A sgRNA is not strictly required, as the use of separate guide RNA and tracrRNA molecules which connect to each other via basepairing is also considered.

The term “antisense polynucleotide” refers to a DNA or RNA, or combination thereof, molecule that is complementary to at least a portion of a specific mRNA molecule capable of interfering with a post-transcriptional event such as mRNA translation. Antisense molecules may include sequences that correspond to the structural genes or for sequences that effect control over the gene expression or splicing event.

The term small interfering RNA (siRNA), short hairping RNA (shRNA), and double stranded RNA (dsRNA) as used herein refer to RNA molecules utilized in RNA interference (RNAi). Methods of RNAi in fungi are known, see e.g. Dang et al. 2011.

dsRNA contains a sequence that is essentially identical to the mRNA of the gene of interest or part thereof. The presence of the double stranded molecule results in the destruction both the double stranded RNA and also the homologous RNA transcript from the fungus gene, efficiently reducing or eliminating the activity of the target gene. siRNA molecules comprise a double stranded nucleotide sequence that is identical to about 19-21 contiguous nucleotides of the target mRNA.

shRNA is a sequence of RNA that makes a tight hairpin turn that can be used to silence target gene expression. shRNA are incorporated into the RNA-interfering silencing complex (RISC) for activity. The incorporated RISC complex is then directed to mRNA that has a complementary sequence to the shRNA. In the case of perfect complementarily, RISC cleaves the mRNA. In the case of imperfect complementarily, RISC represses translation of the mRNA.

A gene knockout cassette as used herein comprises: a promoter sequence, an open reading frame, and a 3′ untranslated region, and can comprise any of the nucleotide sequences of the present invention.

The CRISPR systems described herein may utilize a mature tracrRNA complex that directs Cas9 to the target DNA via Watson-Crick base-pairing between the spacer on the RNA and the protospacer on the target DNA next to the protospacer adjacent motif (PAM), an additional requirement for target recognition. Cas9 then mediates cleavage of target DNA to create a double-stranded break within the protospacer. A skilled artisan will appreciate that each of the engineered guide RNA (gRNA) of the present invention is further designed such as to associate with a target genomic DNA sequence of interest next to a protospacer adjacent motif (PAM), e.g., a PAM matching the sequence relevant for the type of Cas9 utilized. In some embodiments, a RNA-guided DNA nuclease e.g., a CRISPR nuclease, may be used to cause a DNA break at a desired location in the genome of a cell.

CRISPR systems that may be used in the practice of the invention vary greatly. CRISPR systems can be a type I, a type II, or a type III system. Non-limiting examples of suitable CRISPR proteins include Cas3, Cas4, Cas5, Cas5e (or CasD), Cas6, Cas6e, Cas6f, Cas7, Cas8a1, Cas8a2, Cas8b, Cas8c, Cas9, Cas10, Casl Od, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cul966.

In certain embodiments, the CRIPSR nuclease may be a “functional derivative” of a naturally occurring Cas protein. A “functional derivative” of a native sequence polypeptide is a compound having a qualitative biological property in common with a native sequence polypeptide. “Functional derivatives” include, but are not limited to, fragments of a native sequence and derivatives of a native sequence polypeptide and its fragments, provided that they have a biological activity in common with a corresponding native sequence polypeptide. A biological activity contemplated herein is the ability of the functional derivative to hydrolyze a DNA substrate into fragments. The term “derivative” encompasses both amino acid sequence variants of polypeptide, covalent modifications, and fusions thereof.

Cleaved genes of the instant invention may be further subjected to insertion or deletion (indel) by an error prone non-homologous end joining (NHEJ) mechanism, generating a frameshift in the gene's sequence. In some embodiments, the generated frameshift results in inactivation or knockout of the gene. In some embodiments, the generated frameshift creates an early stop codon in the gene and results in generation of a truncated protein. In such embodiments, the method results in the generation of a truncated protein encoded by the gene and a functional protein encoded by the functional allele. In some embodiments, a frameshift generated in a gene using the methods of the invention results in nonsense-mediated mRNA decay of the transcript of the mutant allele.

RNA molecules of the instant invention may comprise one or more chemical modifications which imparts a new or improved property (e.g., improved stability from degradation, improved hybridization energetics, or improved binding properties with an RNA guided DNA nuclease). Suitable chemical modifications include, but are not limited to: modified bases, modified sugar moieties, or modified inter-nucleoside linkages. Non-limiting examples of suitable chemical modifications include: 4-acetylcytidine, 5-(carboxyhydroxymethyl)uridine, 2′-O-methylcytidine, 5-carboxymethylaminomethyl-2-thiouridine, 5-carboxymethylaminomethyluridine, dihydrouridine, 2′-O-methylpseudouridine, “beta, D-galactosylqueuosine”, 2′-O-methylguanosine, inosine, N6-isopentenyladenosine, 1-methyladenosine, 1-methylpseudouridine, 1-methylguanosine, 1-methylinosine, “2,2-dimethylguanosine”, 2-methyladenosine, 2-methylguanosine, 3-methylcytidine, 5-methylcytidine, N6-methyladenosine, 7-methylguanosine, 5-methylaminomethyluridine, 5-methoxyaminomethyl-2-thiouridine, “beta, D-mannosylqueuosine”, 5-methoxycarbonylmethyl-2-thiouridine, 5-methoxycarbonylmethyluridine, 5-methoxyuridine, 2-methylthio-N6-isopentenyladenosine, N-((9-beta-D-ribofuranosyl-2-methylthiopurine-6-yl)carbamoyl)threonine, N-((9-beta-D-ribofuranosylpurine-6-yl)N-methylcarbamoyl) threonine, uridine-5-oxyacetic acid-methylester, uridine-5-oxyacetic acid, wybutoxosine, queuosine, 2-thiocytidine, 5-methyl-2-thiouridine, 2-thiouridine, 4-thiouridine, 5-methyluridine, N-((9-beta-D-ribofuranosylpurine-6-yl)-carbamoyl)threonine, 2′-O-methyl-5-methyluridine, 2′-O-methyluridine, wybutosine, “3-(3-amino-3-carboxy-propyl)uridine, (acp3)u”, 2′-O-methyl (M), 3′-phosphorothioate (MS), 3′-thioPACE (MSP), pseudouridine, or 1-methyl pseudo-uridine. Each possibility represents a separate embodiment of the present invention. RNA compositions described herein may be delivered to a target cell by any suitable means.

Any suitable viral vector system may be used to deliver nucleic acid compositions e.g., the RNA compositions of the subject invention.

Conventional viral and non-viral based gene transfer methods can be used to introduce nucleic acids and target tissues. In certain embodiments, nucleic acids are administered for in vivo or ex vivo gene therapy uses. Non-viral vector delivery systems include naked nucleic acid, and nucleic acid complexed with a delivery vehicle such as a liposome or poloxamer.

Methods of non-viral delivery of nucleic acids and/or proteins include electroporation, lipofection, microinjection, biolistics, particle gun acceleration, virosomes, liposomes, immunoliposomes, lipid nanoparticles (LNPs), polycation or lipid:nucleic acid conjugates, artificial virions, and agent-enhanced uptake of nucleic acids or can be delivered to fungi cells by bacteria or viruses.

The use of RNA or DNA viral based systems for viral mediated delivery of nucleic acids take advantage of highly evolved processes for targeting a virus to specific cells in the fungus and trafficking the viral payload to the nucleus. Conventional viral based systems for the delivery of nucleic acids include, but are not limited to, retroviral, lentivirus, adenoviral, adeno-associated, vaccinia and herpes simplex virus vectors for gene transfer.

Experimental Details

Materials and Methods

Strains, Plasmids, and Media

The strains used in this study are Cryptococcus neoformans var. grubii strain H99 as wildtype (WT) and S. cerevisiae BY4741. Bacterial strain used was Escherichia coli DH5-α™ Max Efficiency® (Invitrogen, Carlsbad, Calif.) as competent cells. Plasmid pCR topo 2.1 was used for cloning and biolistic transformation. Cloning was carried out using TOPO® TA Cloning® Kit, with pCR™2.1-TOPO® (Invitrogen, Carlsbad, Calif.). The three putative ceramide synthase genes in this study have the following identifiers: CNAG_06717 (Genbank accession number XM_012192296), CNAG_02086 (Genbank accession number XM_012194542), CNAG_02087 (Genbank accession number XM_012194543). For overexpression studies, pYES2/CT was used for expression of CNAG_06717 in S. cerevisiae BY4741 while pRS425 was used to express CNAG_02086 and CNAG_02087. Cn strains were routinely grown in YPD broth at 30° C. and 0.04% CO₂ for 20-22 hours with shaking at 225 rpm. Dulbecco's modified eagle medium (DMEM) buffered with 25 mM HEPES (pH 4.0 or pH 7.4) was used to grow Cn at 37° C. in the presence of 5% CO₂ (physiologically relevant conditions). S. cerevisiae transformed with pYES2/CT was grown in YNB without amino acids, 1 g/L amino acid mixture lacking uracil (ura−), 5 g Ammonium sulfate, 0.4 g NaPO4 dibasic, and 2% Glucose or 1% Galactose+1% Raffinose (to induce expression). Similarly, S. cerevisiae transformed with pRS425 was grown in synthetic leucine (leu-) dropout media. Strains containing both vectors were grown in synthetic leu-ura− dropout media. Bacterial strains were grown at 37° C. in Luria-Bertani media containing 75 mg/L of ampicillin (Sigma). All primers are specified in Table 1.

TABLE 1
Primers used in this study.

| Number | Name | Sequence 5′-3′ |
|---|---|---|
| 1 | CNAG_067175UTRF | CTGGATCCGCGTCAAGTGGGTATTTCGT |
| 2 | CNAG_067175UTRR | CTACTAGTAGCTCGTGGGTGTTTGGTTA |
| 3 | CNAG_067173UTRF | CTGATATCTCTTGGATAGCCTGCGACTT |
| 4 | CNAG_067173UTRR | CTGGGCCCGACGTCAGGAAGCCTTTGTC |
| 5 | CNAG_0202865UTRF | CTGGATCCGGCCGTGAAGAGGAATAACA |
| 6 | CNAG_020865UTRR | CTACTAGTGTTGTCGAGATGTGGCTGAA |
| 7 | CNAG_020863UTRF | CTCTCGAGGGTGATCGTGGCTTGCTT |
| 8 | CNAG_020863UTRR | CTGGGCCCTAGCTGTTCTACGTCAAGTGGTC |
| 9 | CNAG_020875UTRF | CTGGATCCGGTGATCGTGGCTTGCTT |
| 10 | CNAG_020875UTRR | CTACTAGTTAGCTGTTCTACGTCAAGTGGTC |
| 11 | CNAG_020873UTRF | CTCTCGAGCAGGTGATGCACCGTGAGA |
| 12 | CNAG_020873UTRR | CTGGGCCCGAAGGACCTTTCCCAACTCC |
| 13 | pRS425_Ahomo_gal1p | ccctcgaggtcgacggtatcgataagcttgatatcgaattcctg |
| | | cagccc |
| | | GATCCACTAGTACGGATTAGAAGCC |
| 14 | pYES/ct_86homo | GACCTTCGCCGATGGCTGGGCTTATTTGGTGTCACTGCTGGAC |
| | | CGGGCAT GTTTTTTCTCCTTGACGTTAAAGTATAGAG |
| 15 | pYES/ct_87homo | GTTACACTGGACGCTCGCCTTCGGTGAAGATGTTTATGCCTTT |
| | | GGGACAT GTTTTTTCTCCTTGACGTTAAAGTATAGAG |
| 16 | 86_ATG_fwd | ATGCCCGGTCCAGCAGTG |
| 17 | 87_ATG_fwd | ATGTCCCAAAGGCATAAACATCTTC |
| 18 | 86_TAA_pRS425Homo | agctggagctccaccgcggtggcggccgctctagaactagtgga |
| | | tccccc |
| | | TTACTCAGCCTTCACCTTCACTTC |
| 19 | 87_TGA_pRS425Homo | agctggagctccaccgcggtggcggccgctctagaactagtgga |
| | | tccccc |
| | | TCATTCTTCCTTCGCTTCATCC |
| 20 | IDTMMREC67F | ACATCACACTGCGGCCTCATCGTGCCTCTCCTTTTC |
| 21 | IDTMMREC67R | GTCAAGCTAAGCGGCCCAAGTCGCAGGCTATCCAA |
| 22 | MMREC86F | ACATCACACTGCGGCCGCGGCCGTGAAGAGGAATAACA |
| 23 | MMREC86R | GTCAAGCTAAGCGGCCGCCGGAAACATCACTCAAGCAA |
| 24 | MMREC87F | ACATCACACTGCGGCCGCGTGATCGTGGCTTGCTTGAG |
| 25 | MMREC87R | GTCAAGCTAAGCGGCCGCCTATCCGTCTACTGAACGATTA |

Isolation and Cloning of C. neoformans Ceramide Synthase Genes

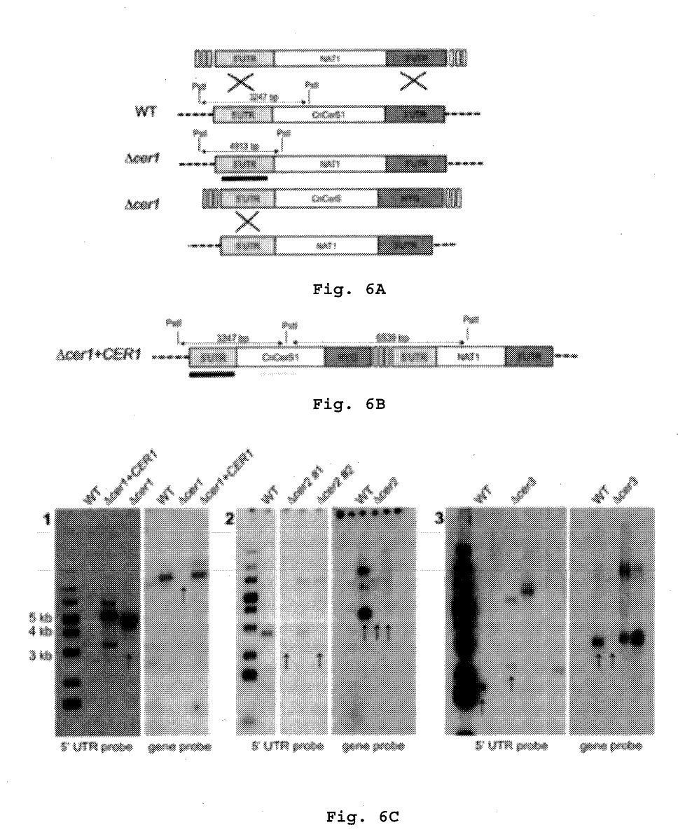

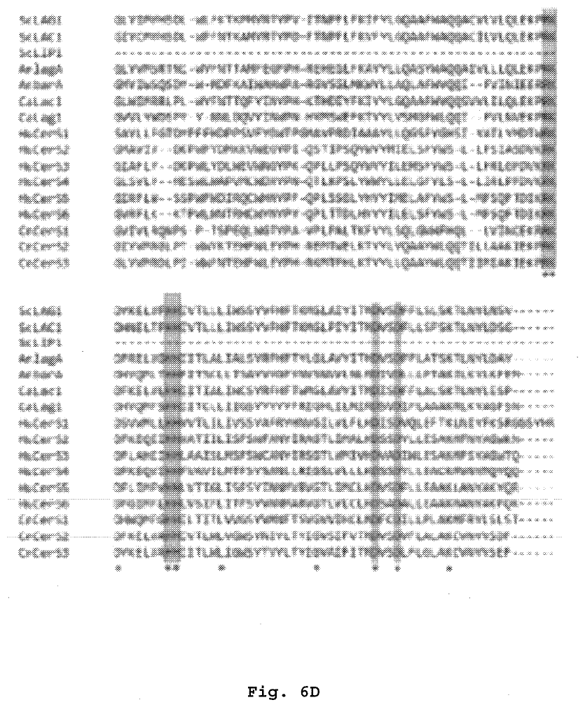

To independently delete each of the three ceramide synthase genes from the genome of C. neoformans, plasmids using nourseothricin acetyltransferase (NAT1) (Werner BioAgents, Germany) selectable marker deletion strategy were constructed. Each NAT1 deletion plasmid contained 1.5 kilobases (Kb) of 5′ untranslated region (UTR) upstream of the ORF as well as 1.5 kilobases of the 3′UTR downstream of the ORF. Generally, the 5′UTR and 3′UTR of the gene of interest was constructed flanking NAT1 gene, whose expression is under the control of actin promoter. The 5′UTR and 3′UTR were generated by PCR using specific primers containing restriction sites on genomic C. neoformans H99 DNA. These fragments were then cloned into pCR2.1 TOPO vector generating plasmids pCR-5UTR and pCR-3UTR and sequenced for each of the three genes of interest (Cer1-5′UTR, Cer1-3′UTR, Cer2-5′UTR, Cer2-3′UTR. Cer3-5′UTR, Cer3-3′UTR). The 3′UTR was then sub-cloned into plasmid pCR-NAT1 vector, generating plasmid pCR-3UTR:NAT1. The 5′UTR was subcloned into pCR-3′UTR::NAT1 generating pCR 5′UTR::NAT1::3′UTR for each ceramide synthase. These constructs were named pΔcer1, pΔcer2, and pΔcer3. C. neoformans wildtype strain H99 was independently transformed with each of the three constructs pΔcer1, pΔcer2, and pΔcer3, by biolistic transformation according to (Singh, Qureshi et al. 2011). Transformants were grown on Yeast peptone dextrose (YPD) plates containing 100 μg/ml of nourseothricin. Resistant colonies were chosen randomly and purified through serial passage on selective media. Correct integration of DNA cassettes was examined by southern blot analysis and performed according to (Singh, Qureshi et al. 2011). Transformants for each ceramide synthase gene, showing deletion of the gene and insertion of the plasmid cassette were obtained and were chosen and designated Δcer1 strain, Δcer2 strain, and Δcer3 strain. To reintroduce the genes back in their respective knockout mutants, reconstituted constructs, pCR-Cer1-ACT-HYG, pCR-Cer2-ACT-HYG, pCR-Cer3-ACT-HYG plasmid constructs were generated as follows: A fragment (4.5 kb) containing the entire ORF of the gene and 1.5 kb of the upstream (5′UTR) was generated by PCR using wildtype H99 genomic DNA as a template and was cloned into the pCR2.1-TOPO vector generating plasmid containing 5′UTR-GENE. This construct was then sub cloned into pSK-ACTIN-HYG plasmid containing Hygromycin resistant marker forming pSK-Cer1-ACT-HYG, pSK-Cer2-ACT-HYG, pSK-Cer3-ACT-HYG. The Δcer1, Δcer2, and Δcer3 mutant strains were each transformed with pSK-Cer1-ACT-HYG, pSK-Cer2-ACT-HYG, and pSK-Cer3-ACT-HYG plasmid respectively using biolistic delivery of DNA using the scheme as shown in FIGS. 6A-6D. Transformants were grown on YPD plates containing 100 μg/ml of hygromycin B. Stable transformants were selected, grown on YPD, followed by extraction of DNA and confirmation with southern blot using gene sequence probes. These reconstituted strains were named Δcer1+CER1, Δcer2+CER2, and Δcer3+CER3.

Phylogenetic Analysis of Putative Ceramide Synthase Genes

Representative fungal ceramide synthase genes and the three computationally annotated ceramide synthase genes were aligned with ClustalOmega using default parameters. Exact maximum likelihood phlyogentic tree construction was performed using TreePuzzle (Schmidt, Strimmer et al. 2002, Schmidt and von Haeseler 2007) with 1000 quartet puzzling steps. Human ceramide synthase 1 was selected as the outgroup, with the exact neighbor-joining tree method used to find the parameter estimates. The Meuller-Vingron model of substitution was used to calculate mismatch penalties. Dendroscope (Huson, Richter et al. 2007, Huson and Scornavacca 2012) was used to visualize the resulting phylogenetic tree.

Biochemical Characterization of Cn Ceramide Synthases

Cn ceramide synthase enzymes were further biochemically characterized using a fluorescent assay using different combinations of substrates and buffer pH. Specifically, NBD-sphingosine, NBD-phytosphingosine were used to check for formation of ceramides and phytoceramides. Fatty Acyl CoA chain lengths C18, C24 and C26 were used tested for chain length specificity. pH dependence of ceramide synthase activity was assessed by using a range of buffers from 3.0-10.0. For all further assays, three buffers: Sodium acetate trihydrate, CH₃COONa.3H₂O, Acetic acid NaOAc buffer (pH 4.0), Na₂HPO₄—NaH₂PO₄ (pH 7.0) and Sodium Carbonate Na₂CO₃. 10H₂O—Sodium Bicarbonate NaHCO₃ (pH 10.0) were used.

Generation of S. cerevisiae Strains Expressing Cn Ceramide Synthases

3 plasmids were constructed respectively for the genes CNAG_06717, CNAG_02086 and CNAG_02087. Gene fragment for CNAG_06717 containing V5 and 6× histidine tags and overlapping ends with pYES2/CT vector was generated using IDT gblocks gene fragments. The construct was inserted into vector pYES2/CT by plasmid gap repair using the gene fragment with flanking homology to the linearized plasmid vector. Positive colonies were purified through serial passage on ura− media. The resulting colonies were sequenced to confirm correct integration. Similarly, plasmid pRS425 was used to insert genes CNAG_02086, and CNAG_02087. These plasmids do not contain a Gal1 promoter. Therefore, the Gal1p was amplified from plasmid pYES2/CT. Primers were designed to amplify Gal1p with overlap of part of plasmid pRS425, gene CNAG-02086 ATG forward primer, CNAG_02086 with TAA and overlap of pRS425, as well as CNAG-02087 ATG forward primer, CNAG 02087 with TAA and overlap of pRS425. These fragments were then co-transformed along with the linearized vector into Sc BY4741. Transformants were selected on synthetic leu− dropout media. Two additional strains were constructed by cotransforming pYES2/CT+Cer1 along with pRS425+Cer2 or pRS425+Cer3. These transformants were passaged on synthetic leu-ura− media to obtain pure isolates.

Protein Microsomal Preparation

Microsomal isolation method was adapted from (Ternes, Wobbe et al. 2011) with modifications. Briefly, cells of S. cerevisiae strain BY4741 expressing gene of interest (Cer1, Cer2, Cer3, Cer1+Cer2, or Cer1+Cer3) were grown in 10 ml YNB (containing the appropriate amino acid dropout mix)+2% glucose, overnight at 30° C. The next day, these cells were washed twice with PBS and transferred to 300 ml YNB+1% galactose+1% raffinose media and allowed to grow overnight. These cells were then centrifuged and the pellet was resuspended in 1-2 ml lysis buffer (20 mM HEPES/KOH pH 7.4, 25 mM KCl, 2 mM MgCl₂, 250 mM sorbitol) and 50 μl protease inhibitor cocktail (Thermo scientific, Waltham, Mass.). ˜1 ml volume of glass beads was added to 1 ml lysate in a tube and this slurry was vortexed vigorously for ˜2 hours. Cell debris were removed by centrifugation at 1000×g, 4° C., for 3 mins. Supernatant was loaded on to a 60% sucrose cushion (w/w), and spun in an ultracentrifuge at 4° C., 24,000 RPM, for 1 hour. The microsomes were isolated from the interphase with a Pasteur pipette and stored at −80° C.

Fluorescent Cryptococcal Ceramide Synthase Assay

An assay for ceramide synthase activity was adapted from (Kim, Qiao et al. 2012, Tidhar, Sims et al. 2015) with minor changes. Microsomes of overexpressed ceramide synthase were used as enzymes for these reactions. Briefly, NBD-Sphingosine or NBD-Phytosphingosine (Avanti Polar lipids, Alabaster, Ala.) was combined with Fatty Acyl CoA of varying chain lengths (C18, C24, C26) (Avanti Polar lipids, Alabaster, Ala.) as a substrate mixture. A 100 μl reaction was carried out using reaction buffer (20 mM Hepes, pH 7.4, 25 mM KCl, 2 mM MgCl₂, 0.5 mM DTT, 0.1% (w/v) fatty acid-free BSA) along with 10 μM NBD sphingosine and 50 μM fatty acyl CoA. 150 μg of microsomal protein was added per reaction, as measured in a Bradford assay (effective protein amount empirically determined). The reactions were then incubated at 35° C. for 90 minutes. The reactions were then stopped with 2:1 chloroform:methanol, followed by gentle vortexing. The lipids were then extracted and dried in a speed vacuum (SPD 2010) followed by resuspension in 100% methanol. The reaction was analyzed by thin layer chromatography, using chloroform/methanol/water (8:1:0.1, v/v/v) as the solvent mixture.

Virulence Studies and Histology Analysis in a Murine Mouse Model of Cryptococcosis

3-4 weeks old female CBA/JCrHsd (Harlan Laboratories, Indianapolis, Ind., USA) mice were used for all experiments. Mice were anesthetized with 60 μl xylazine/ketamine mixture containing 95 mg ketamine and 5 mg xylazine per kilogram of body weight prior to infection. Cn strains WT H99, 4cer1, Δcer2, Δcer3 and Δcer1+CER1 were grown overnight in YPD broth at 30° C. The next day, cells were pelleted, washed twice and resuspended in PBS at a concentration of 3.5×10⁷ cells/ml. For survival studies, ten CBA/JCrHsd mice per strain were infected with 7×10⁵ cells for each strain in a volume of 20 μl through nasal inhalation. For tissue burden analysis, 9 mice per strain were used. Lung, brain, kidney, liver and spleen were excised and homogenized in 10 ml PBS using stomacher 80 (Seward, UK) for 2 min at high speed. Serial dilutions were plated in duplicate on YPD agar plates and incubated for 48-72 hours at 30° C. for assessment of CFU per organ. For histopathology analysis, 3 mice per experimental group were used. Mice organs were fixed in 3.7% formaldehyde in paraffin and stained with haematoxylin and eosin and mucicarmine. Staining was performed in part by McClain Labs (Smithtown, N.Y.), as well as by Research Histology Core at Stony Brook University.

Extraction and Mass Spectrometry Analysis of Yeast Sphingolipids

For extraction of lipids, cells of wildtype, mutant and reconstituted strains were grown overnight in YPD at 30° C. The next day these cells were washed and transferred to DMEM (pH 4.0 or 7.4) and grown in shaking condition at 37° C.+5% CO₂ for about 16 hours. These cells were washed and counted for lipid extraction. Briefly, 108 cells for each replicate were pelleted in a glass tube in which mandala extraction buffer was added and extraction was performed as described in (Mandala, Thornton et al. 1995). Further extraction was performed according to the methods of Bligh and Dyer (Bligh and Dyer 1959). After measuring the dry weights, the samples were subject to base hydrolysis (Clarke and Dawson 1981). The extracts were dried in a centrifuge under vacuum (SPD 2010, ThermoFisher Scientific, Waltham, Mass.). All internal standards were added prior to lipid extraction. The following internal standards from Avanti Polar Lipids (Alabaster, Ala.) were used: Sphingosine (d17:1), D-erythro-sphingosine (C17 base), N-08:0 Phytosphingosine (N-octanoyl-4-hydroxysphinganine)(Saccharomyces cerevisiae), sphinganine (d17:0) D-erythro-sphinganine (C17 base), Sphingosine-1-Phosphate (d17:1) D-erythro-sphingosine-1-phosphate (C17 base) and C17 Ceramide (d18:1/17:0)N-heptadecanoyl-D-erythro-sphingosine. For the mass spectrometry analysis, the dried extracts were separated on a Thermo Accela HPLC system (San Jose, Calif.) after dissolving in 150 μL of ammonium formate (1 mM) with 0.2% formic acid in methanol. A Peeke Scientific Spectra C8 (Redwood City, Calif.) HPLC column (150×3 mm) into which 10 μl samples were injected. The buffers used for the runs were as follows: Buffer A (2 mM ammonium formate and 0.2% formic acid (FA)) and buffer B, ammonium formate (1 mM) with 0.2% FA in methanol. A gradient using buffer A and B was used, starting with 70% B with an increase to 90% over 5 minutes, followed by a ramp to 99% B over 9 minutes. The column was equilibrated with initial conditions for 8 minutes at a flow rate of 500 μL/min. The HPLC was coupled to the HESI source of a Thermo TSQ Quantum Ultra triple quadrupole mass spectrometer (San Jose, Calif.). The sphingolipid profile was performed using positive ion mode. With the high voltage set to 3.5 kV, vaporizer temperature at 400° C., sheath gas pressure at 60, auxiliary gas pressure at 15 and a capillary temperature of 300° C. The collision cell was operated at 1.5 mTorr of argon. For the duration of the run, transitions for each lipid species were monitored at 100 ms or 50 ms dwell time. 20 lipid standards for our profile from Avanti (Alabaster, Ala.) were used to develop calibration curves and these curves were then used for lipids species to be monitored. Processing of the samples was done using Thermo Xcalibur 2.2 Quan Browser software and exported to excel for reporting results.

In Vitro Growth Studies

From overnight YPD broth cultures of Cn WT, Δcer1 and Δcer1+CER1 were washed twice in phosphate buffered saline (PBS), resuspended and diluted into 10 ml DMEM (buffered with HEPES, pH 4.0 or pH 7.4) to a final density of 104 cells/ml and incubated in shaker incubator at 37° C. with 5% CO₂. Aliquots were taken at time points indicated and serial dilutions were plated on YPD agar for assessment of CFU. For cell wall stability, cells were spotted in serial dilutions on YPD plates with 0.05% SDS. For osmotic stress, cells were spotted on YPD containing 2 mM H₂O₂.

Transmission Electron Microscopy

Cn strains were grown overnight in YPD at 30° C. with shaking. The next day, these cells were washed, counted and transferred to DMEM (pH 4.0 or 7.4) and grown in shaking condition at 37° C.+5% CO₂ to mimic physiological conditions. After 24 hours of growth, these cells were washed with phosphate buffered saline, and fixed in 3% EM grade glutaraldehyde solution for 2 hours. For supplementation experiments, cells grown in physiological condition as mentioned earlier were supplemented with 50 μM ceramides mix (Matreya LLC, PA). For sample preparation, after glutaraldehyde fixation, cells were rinsed in 0.1M phosphate buffer pH 7.4 and dispersed and embedded in ultra-low gelling temperature agarose. Tubes containing these cells were then cooled and agarose samples were chopped into cubes of smaller size. Post fixation of these samples was done by rinsing with aqueous potassium permanganate, and then further rinsed and treated with sodium meta periodate. This was followed by another rinse, and ultimately dehydrated through a graded ethanol series. After dehydration samples were embedded in Spurr's resin and polymerized in a 60° C. oven. For sectioning, ultrathin sections of 80 nm were cut with a Leica EM UC7 ultramicrotome and placed on 300 mesh copper grids. Sections were then counterstained with uranyl acetate and lead citrate and viewed with a FEI TeCnail2 BioTwinG2 transmission electron microscope. Digital images were acquired with an AMT XR-60 CCD Digital Camera system.

Replicative Lifespan Studies

Replicative lifespan for WT and mutant Cn strains was measured by microdissection according to (Park, McVey et al. 2002, Bouklas, Jain et al. 2017) with minor adjustments. Briefly, Cells of Cn were plated and incubated at 37° C. The bud of these cells were followed by identifying the first bud and following its increase in size during the cell cycle. The daughter cells were separated from mother cell at the end of each division (1-2 hours) with the help of a 50 μm fiber optic needle (Cora Styles) on a tetrad dissection Axioscope A1 microscope (Zeiss) at 100× magnification. Replicative lifespan of each cell was determined as sum of the total buds until the mother cells fail to divide any further.

Glucose Dependent Medium Acidification to Measure Plasma Membrane H+-ATPase

Glucose-dependent medium acidification was monitored by a modification of a procedure described previously (Perlin, Brown et al. 1988, Soteropoulos, Vaz et al. 2000). Cultures of Cn strain WT, Δcer1, Δcer1+CER1, Δgcs1 were grown to mid-log Phase in YPD. The next day, these cells were transferred to DMEM at pH 4.0 and allowed to grow under shaking conditions for 24 hours. These cells were then harvested and washed using 100 mM KCl, pH 5.0. These pellets were then resuspended in 10 ml KCl, pH 5.0 and incubated under shaking condition at 30° C. These samples were then stored at 4° C. overnight prior to use. For the assay, cells were concentrated to a final A590 of approximately 2.0. 20 μl cells along with 155 μl of bromophenolblue (50 μg/ml) in 100 mM KCl, pH 5.0. 20 μl 20% (w/v) glucose was added to initiate the reaction. Medium acidification was monitored at 590 nm over a period of 5 hours (data point every 3 mins) in a microplate reader (SpectraMax M5).

Drug Design Experiments: Library Preparation

The compounds were first diluted to 1 mM each (1:10 dilution with 10% DMSO) with a physiological buffer (YNB medium buffered with HEPES at pH 7.4 containing 2% glucose) and subsequently diluted to 300 μM (1:3.3 dilution) with the same medium (3% DMSO). A 100-μl aliquot of this solution was placed into each well of a 96-well master plate and stored at −20° C. until use. Each well in the screening master plates contained 10 compounds at 1 μM each. The ceramide synthase reaction was performed in a 96-well plate format using fungal Cer1 (Cn Cer1) or human ceramide synthase (Hu-Cer1, -Cer2, -Cer3, -Cer4, -Cer5, or -Cer6) enzyme, containing NBD-Sphingosine (NBD-Sph) (Avanti Polar Lipids) and 18:0 Coenzyme A (Stearoyl Coenzyme A, Avanti Polar Lipids) as substrates using the following concentrations: 10 μM NBD-Sph and 50 μM fatty acid CoA. The reaction mixture was prepared in HEPES buffer (20 mM HEPES, pH 7.2, 25 mM KCl, 250 mM sucrose, and 2 mM MgCl₂). Library compounds were added and plate incubated for 1 hour at 37 C. The lipid product (NBD-ceramide) was separated using solid phase extraction (SPE) column chromatography using Strata® C18-E, 96 well plates (Phenomenex). Reaction product was measured using a plate reader.

Enzyme Preparations

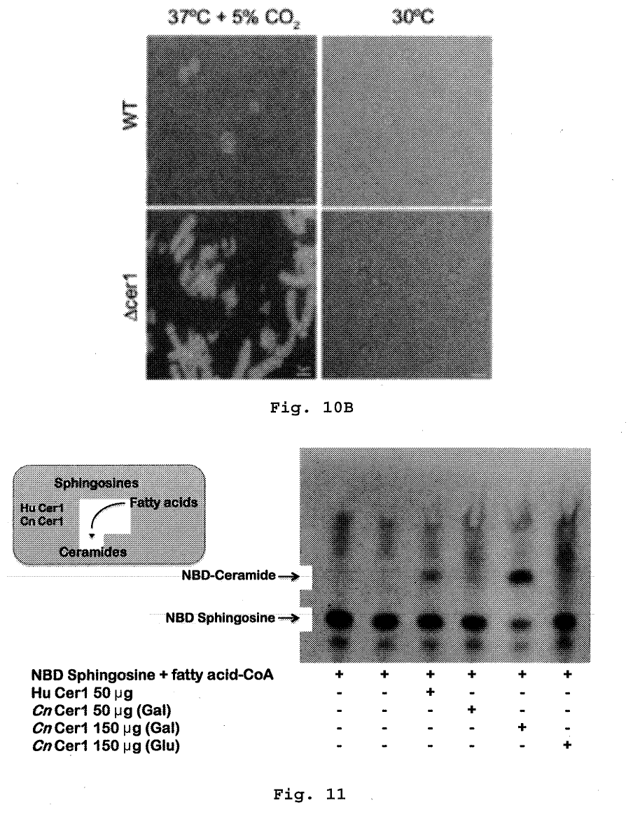

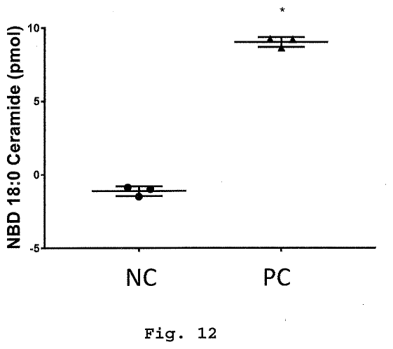

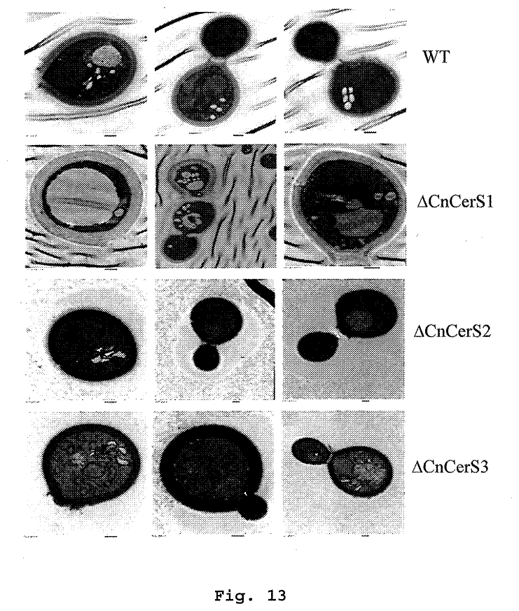

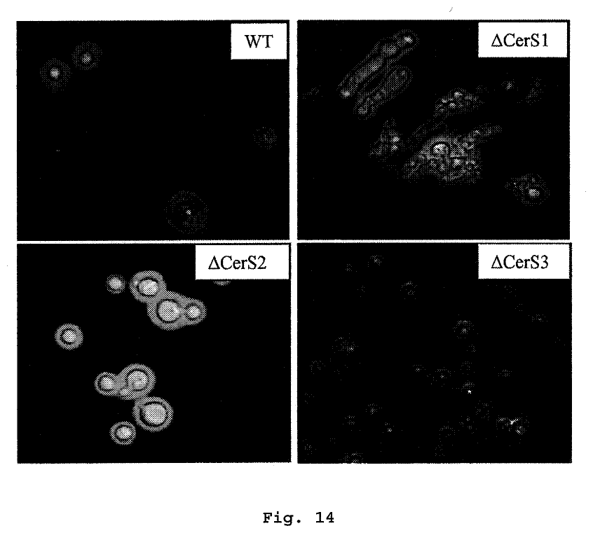

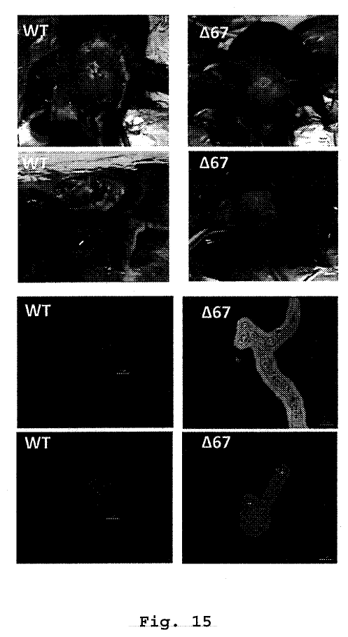

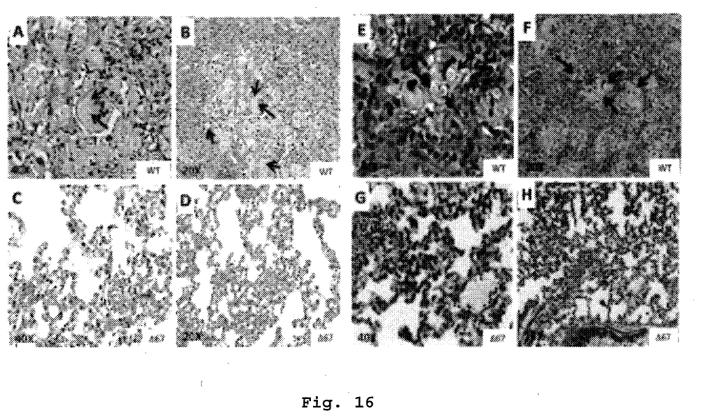

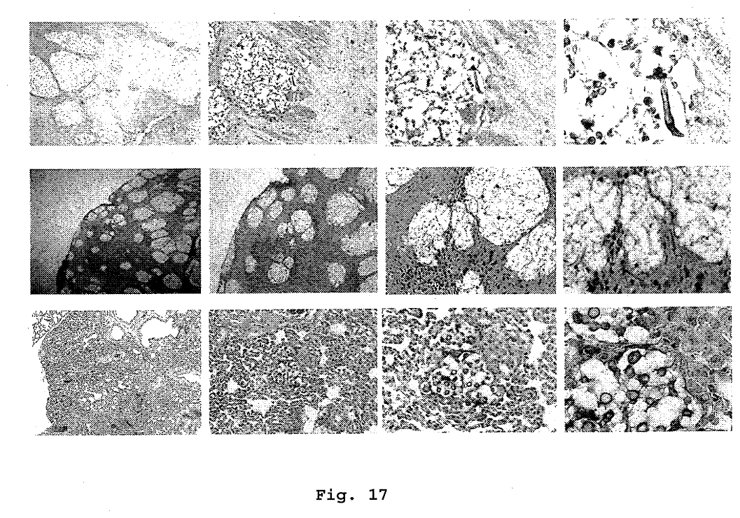

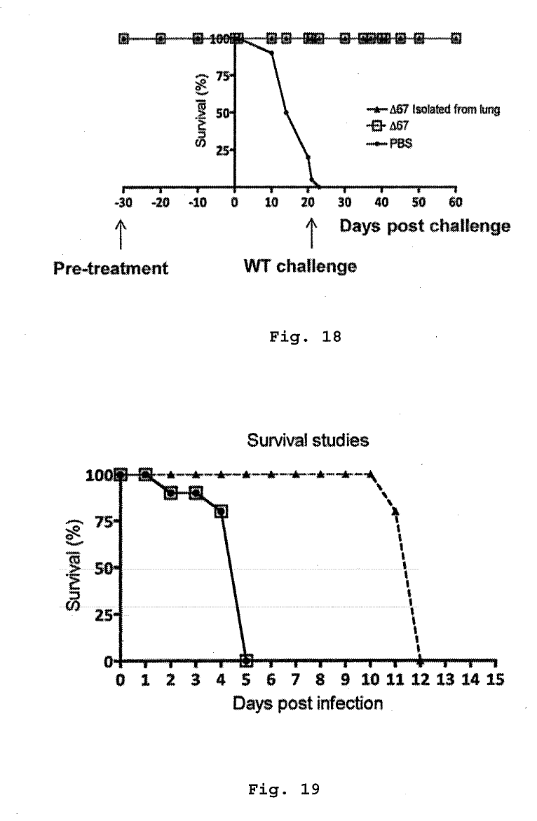

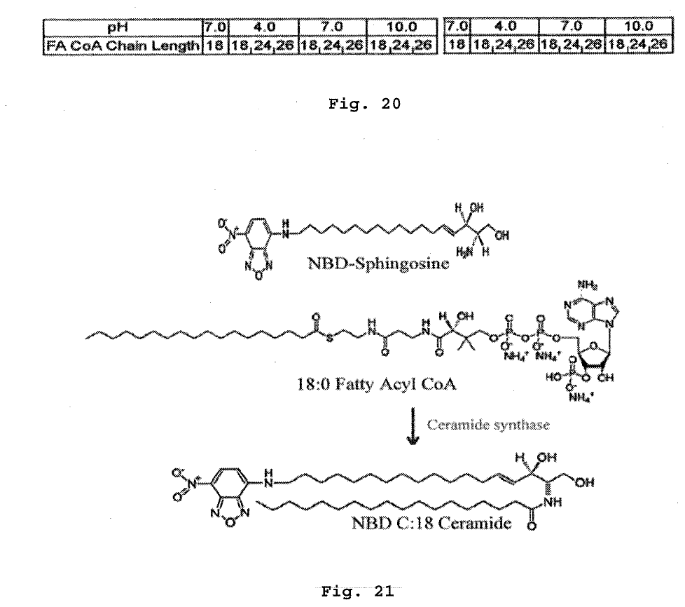

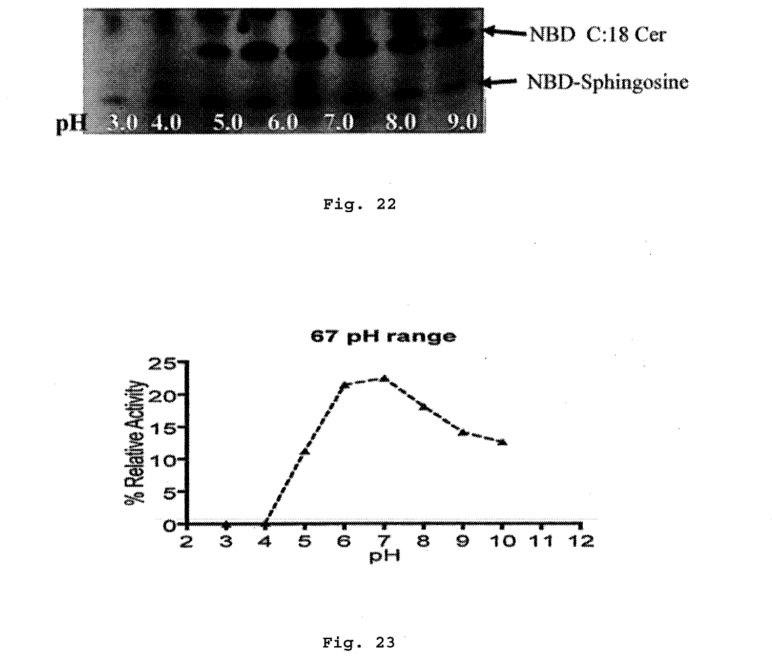

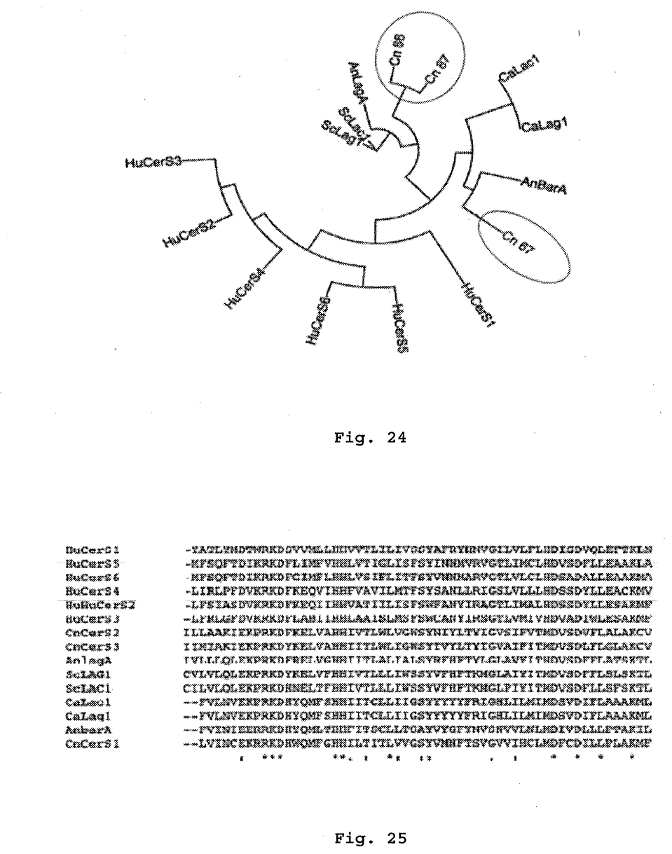

The Cn Cer1 enzyme is expressed using the pYES-GAL1 galactose inducible system in Saccharomyces cerevisiae whereas the mammalian ceramide synthases (Cer1-6) are induced with tetracycline using the Tet-On System in HCT-116 cells under the control of Geneticin and Blasticidin. Cn Cer1 or Hu Cer1-6 enzymes were extracted using the tag system and enriched in microsomal preparation. Before using the enzymes in the plate screening, each preparation was tested for ceramide activity using the TLC assay illustrated in FIG. 11, to make sure the ceramide synthase activity works as expected. Appropriate negative controls (e.g. Cn Cer1 on glucose or Hu Cer1 in absence of tetracycline) were included in each plate during the screening.

Antifungal and Cytotoxicity Assays

The ceramide synthase assay was performed using NBD-Sphingosine (NBD-Sph) (Avanti polar lipids) and 18:0 Coenzyme A (Stearoyl Coenzyme A, Avanti polar lipids) as substrates using the following concentrations: 10 μM NBD-Sph and 50 μM fatty acid CoA. Enzyme(s)—ceramide synthases—were prepared using microsome purification of the yeasts or mammalian expression system. For yeast, the S. cerevisiae strain BY4741 expressing C. neoformans ceramide synthase 1 using the pYES GAL1 expression system was grown in 200 ml of liquid YNB 2% galactose medium for 16 hours. The same strain grown in the same medium but containing 2% glucose was used as a negative control because the Cn Cer1 gene is under the control of the galactose promoter (GAL1). Cells were then harvested by centrifugation, resuspended in 2 ml of Lysis Buffer (20 mM HEPES/KOH, pH 7.4, 25 mM KCl, 2 mM MgCl₂, 250 mM sorbitol) and 50 μl of proteinase inhibitor mixture (Sigma-Aldrich)/g of cells, and broken by bead-bashing at 4° C. for 1.5 h. Cell debris were removed by centrifuging at 1000 g at 4° C. for 10 min. The supernatant was loaded on a 60% (w/w) sucrose cushion and centrifuged at 100,000 g for 1 h. The microsomes were collected from the interphase, snap frozen in liquid nitrogen, and stored at −80° C. until use.

For the expression of the mammalian ceramide synthase(s) (e.g. Hu Cer1, Cer2, Cer3, Cer4, Cer5 and Cer6), Cer was induced with tetracycline using the Tet-On System in HCT-116 cells under the control of Geneticin and Blasticidin. Cells were grown in McCoy's 5A medium (Life Technologies) supplemented with 10% Tet-approved fetal bovine serum (Clontech), 150 μg/mL Geneticin (Life Technologies), and 10 μg/mL Blasticidin (InvivoGen) at 37° C. and 5% CO₂. Hu Cer1 expression was induced in HCT-116 cells with 0.25 μg/mL of tetracycline for 48 hours. Cells were washed twice with cold phosphate-buffered saline (PBS), harvested by scraping in cold PBS, re-suspended in lysis buffer (20 mM HEPES pH 7.4, 2 mM KCl, 2 mM MgCl₂ 250 mM sucrose, 10 μl protease inhibitor cocktail (Sigma-Aldrich)/1 mL of cells), and lysed via 10 passages through a 28-gauge insulin syringe. Intact cells and nuclei were removed via centrifugation at 1,000×g at 4° C. for 10 min, the mitochondrial enriched fraction was removed via centrifugation at 10,000×g at 4° C. for 10 min. The resulting supernatant was centrifuged at 100,000×g at 4° C. for 1 h to pellet the microsomes. Microsomes were resuspended in HEPES buffer and protein concentration was measured using the Bradford method (Bio-Rad). Each reaction does contain: i) 10 μM NBD-Sph; ii) 50 μM fatty acid CoA: iii) ˜150 μg Cn Cer1 protein in a final volume of 100 μl. Control reactions: i) 10 μM NBD-Sph+50 μM fatty acid CoA only; ii) 10 μM NBD-Sph+50 μM fatty acid CoA+Hu Cer1 or Cn Cer1 microsomes (50 μg protein—galactose); and iii) 10 μM NBD-Sph+50 μM fatty acid+150 μg Cn Cer1 protein expressed in 2% glucose.

Reaction tubes were incubated at 35-37° C. with gentle shaking for 20-120 min. Reactions were stopped with 250 μl chloroform/methanol (2:1). Vortexed thoroughly and centrifuged for 8 minutes at 3000 rpm at room temperature. The lower organic phase was extracted twice and dried in a speed Vac. Pellet was resuspended in 100% methanol. The fluorescent products were resolved by spotting 10 μl on TLC plates (Sigma Aldrich) in chloroform/methanol/water (8:1:0.1). As illustrated in FIG. 11, the separation efficiency of NED-ceramide band from NBD-sphingosine band was very high with no overlap.

The high throughput assays were adapted from the Avanti Ceramide synthase assay kit (https://avantilipids.com/product/640011/). The reactions were first downsized to 20 μl in a 96-well plate format using human ceramide synthases (Cer1, Cer1, Cer3, Cer4, Cer5, or Cer6) along with Cn Cer1. Lipid product (NBD-ceramide) was separated using solid phase extraction (SPE) column chromatography using Strata® C18-E, 96 well plates (Phenomenex). The reaction product was measured using a plate reader, as in FIG. 12. The Z′ score of the assay is 0.80194, which is considered excellent (FIG. 12).

This approach offers the advantage of a short assay time, small amount of biological material (microsomal enzyme preparation) and, most importantly, it eliminates the TLC because the NBD sphinganine will be separated from the newly formed NBD-ceramide, which is detected by fluorescent spectrometry. Importantly, the approach allows for the prompt elimination of any compound(s) targeting Hu Cer (Cer1 through Cer6), as they will be included in the assay along with the Cn Cer1. For initial hit-finding two DIVERSet Screening Libraries from ChemBridge are used: DIVERSet-EXP and the DIVERSet-CL. Combined, these libraries provide a broad pharmacophore space survey while maintaining structural diversity with 100,000 compounds. Of interest, the DIVERSet-EXP is selected from ChemBridge express-pick collection stock of more than 460,000 handcrafted compounds while the DIVERSet-CL is selected from the ChemBridge core library stock of more than 620,000 parallel-synthesized compounds based on novel scaffolds.

### Example 1. Three Genes in C. neoformans Encode Specific Acyl-CoA Dependent Ceramide Synthases

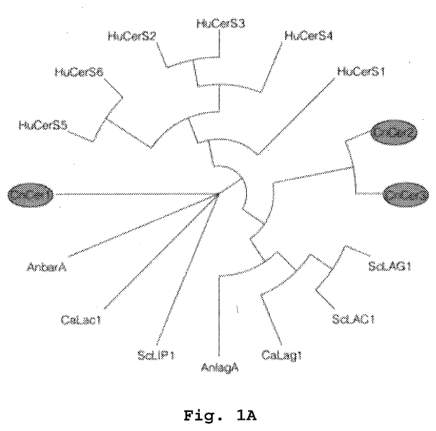

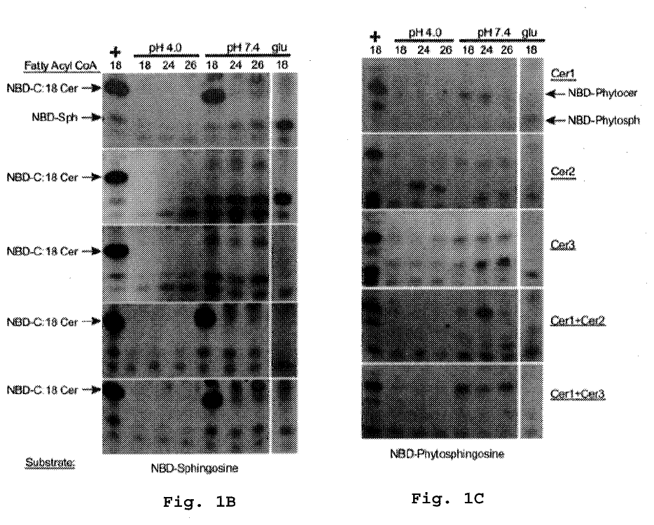

Based on evidence of ceramide synthases in other fungi, a bioinformatic search was performed for putative ceramide synthases present in Cn serotype A H99 (WT). The analysis revealed the presence of three putative ceramide synthases in Cn. The genes CNAG_06717, CNAG_02086, and CNAG_02087 had significant homology to other ceramide synthase genes from A. nidulas, C. albicans, S. cerevisiae as well as H. sapiens (FIG. 1A). They are referred to in this study as Cer1, Cer2, and Cer3, respectively. A phylogenetic analysis shows that Cer2 and Cer3 exhibit high similarity to each other, as well as to ceramide synthases of S. cerevisiae (ScLac1 and ScLag1) and A. nidulans (AnLagA). The Cer1 amino acid sequence is greatly diverged from Cer2 and Cer3, and has partial similarity to ceramide synthases of C. albicans (CaLag1), and A. nidulans (AnBarA). Reports on these genes show a distinct specificity for synthesis of ceramides used for glucosylceramide (GlcCer) synthesis (FIG. 1A) (Li et al., 2006, Rittenour et al., 2011, Cheon et al., 2012). An alignment of the amino acid sequences of these genes revealed conservation of residues that are reportedly important for enzymatic activity (FIG. 6D) (Kageyama-Yahara and Riezman, 2006).

### Example 2. Ceramide Synthase Cer1 Activity In Vitro Shows Preference for C18 Fatty Acyl CoA

To characterize the activity of each enzyme, strains were generated overexpressing each Cn ceramide synthase in S. cerevisiae using either a 6×His or 3×HA tag fused protein. These plasmids were transformed in combination into the S. cerevisiae system to generate strains overexpressing both Cer1-Cer2 or Cer1-Cer3. After induction, the proteins were purified by extraction of microsomes (Ternes et al., 2011). As a negative control, these strains were grown without induction (2% glucose) and were used to control for any S. cerevisiae enzyme activity (FIG. 5A, FIG. 5B).

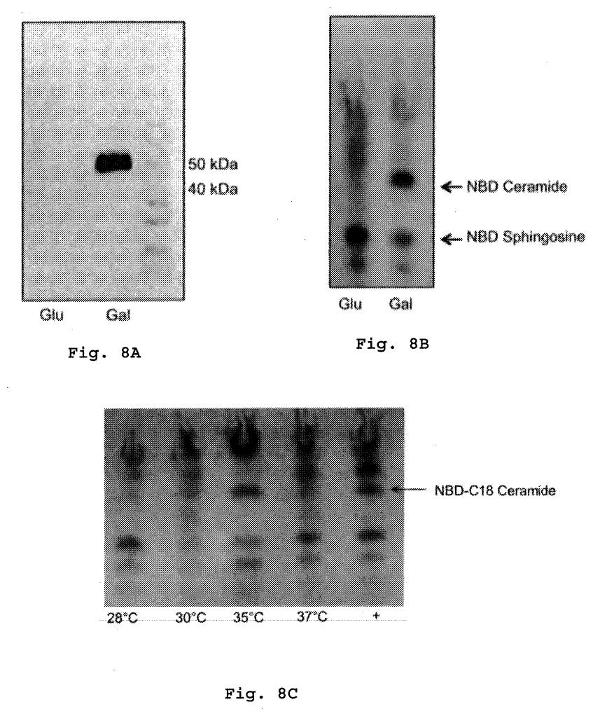

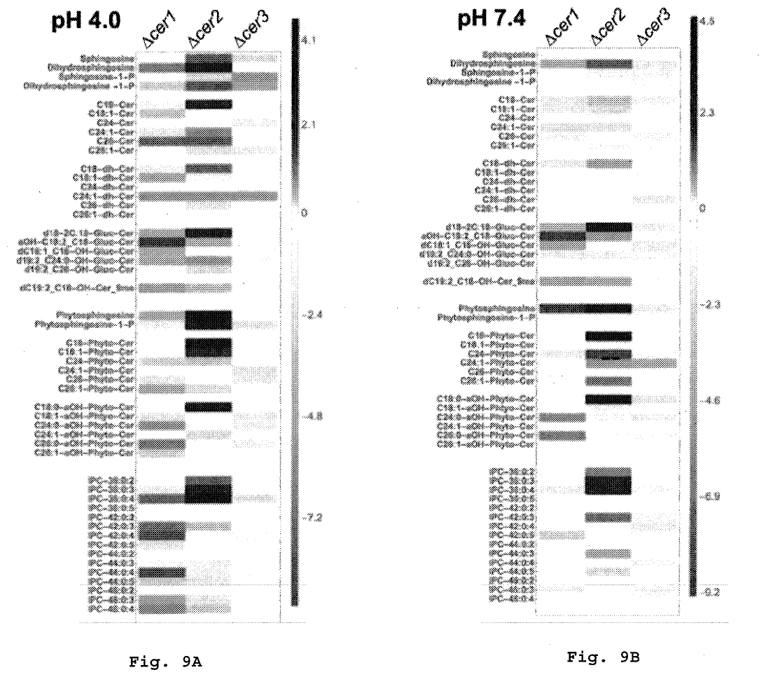

To characterize the properties of the putative ceramide synthases, an in vitro assay for fungal ceramide synthase was developed. Using a fluorescent labeled substrate and fatty acyl CoA, the formation of NBD-ceramide using microsomal Cn ceramide synthase enzyme by thin layer chromatography was investigated. It was observed that the activity of Cer1 is dependent on pH (FIG. 1B, FIG. 1C) and temperature (FIG. 8C). The activity of Cer1 was optimal at pH 7.0 and 35° C.

Each enzyme's specificities were then systematically investigated. Combinations of fatty acyl CoAs, with either sphingosine or phytosphingosine at pH 4.0 or 7.0. Cer1 showed a clear preference for C18 fatty acyl CoA and sphingosine as a substrate (FIG. 1B). When phytosphingosine was used a substrate, the production of C18 and C24 phytoceramide was observed in slightly lower amounts (FIG. 1B). When Cer1 and Cer2 are co-expressed, C24 phytoceramide was the major product. However, when Cer1 and Cer3 are co-expressed, the enzyme activity shifts to C18 and C26 fatty acyl CoA products (FIG. 1C). These results suggest the production of ceramide isoforms is regulated by the stoichiometry of the three ceramide synthase proteins of Cn in the presence of different substrate chain lengths. Uninduced cells (2% glucose) show little to no enzyme activity under these conditions (FIG. 1B, FIG. 1C).

### Example 3. Ceramide Synthases are Important for the Virulence of C. neoformans

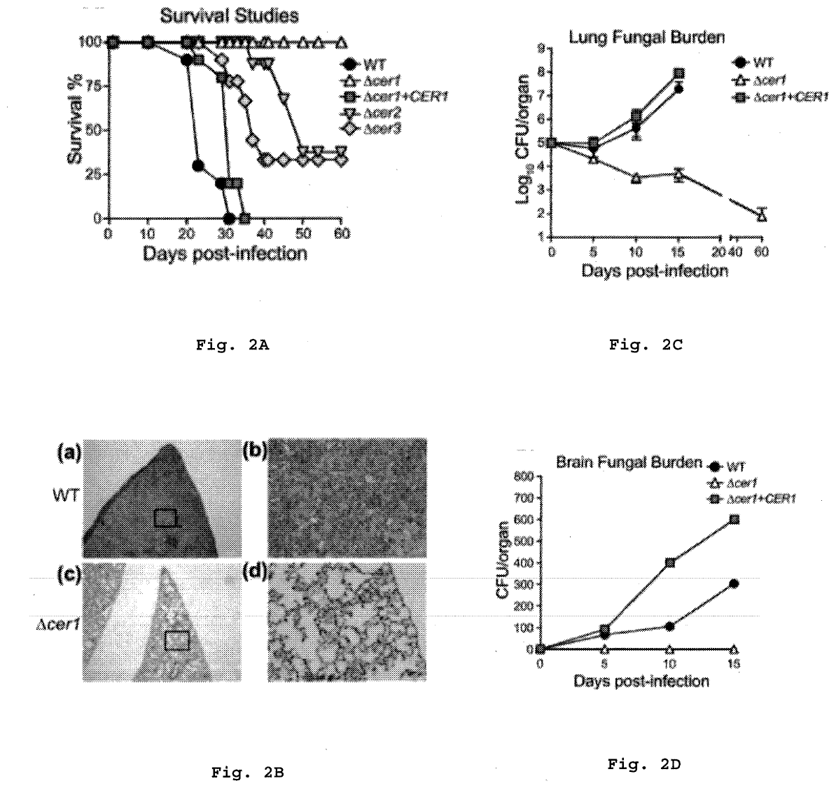

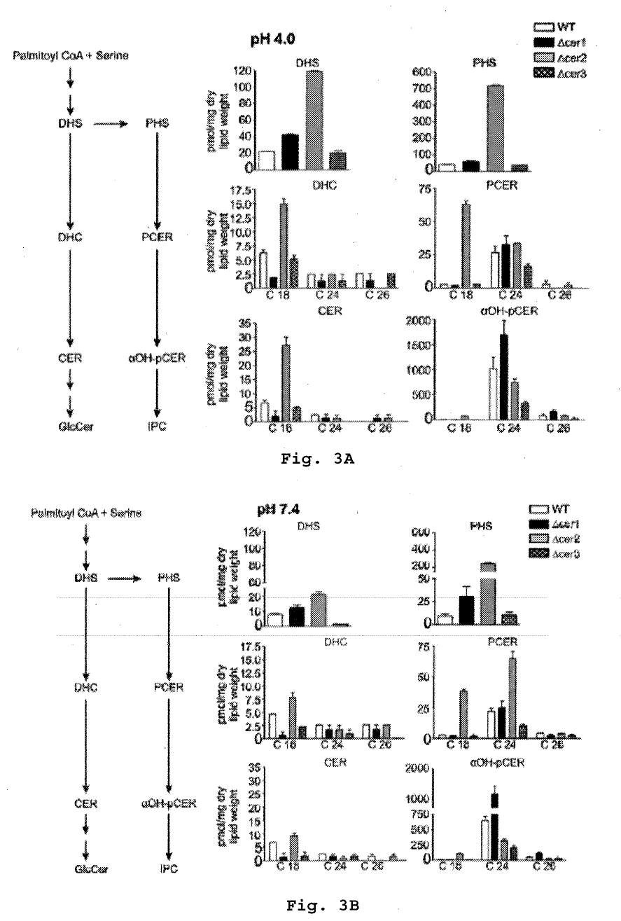

To determine the effect of each ceramide synthase on the virulence of Cn, ceramide synthase deletion strains were created in the WT Cn background (FIGS. 6A, 6B, 6C). Each cell line, containing a deletion cassette or a reintroduced ceramide synthase gene, was tested on CBA/JCrHsd immunocompetent mice. Mice were infected with a normally lethal dose of fungal cells (7×10⁵ cells) intranasally to establish cryptococcosis, and were monitored for survival. It was discovered that the average survival of mice infected with WT Cn was 25±6 days, while all mice infected with Δcer1 survived (60 days of observation) (FIG. 2A). The average survival of mice infected with the reconstructed gene strain, Δcer1+CER1, experienced mortality similar to the WT control, with an average survival of 26±7 days (FIG. 2A). Mice infected with Δcer2 and Δcer3 showed a survival pattern distinct from WT (FIG. 2A), with each deletion causing ˜70% mortality.

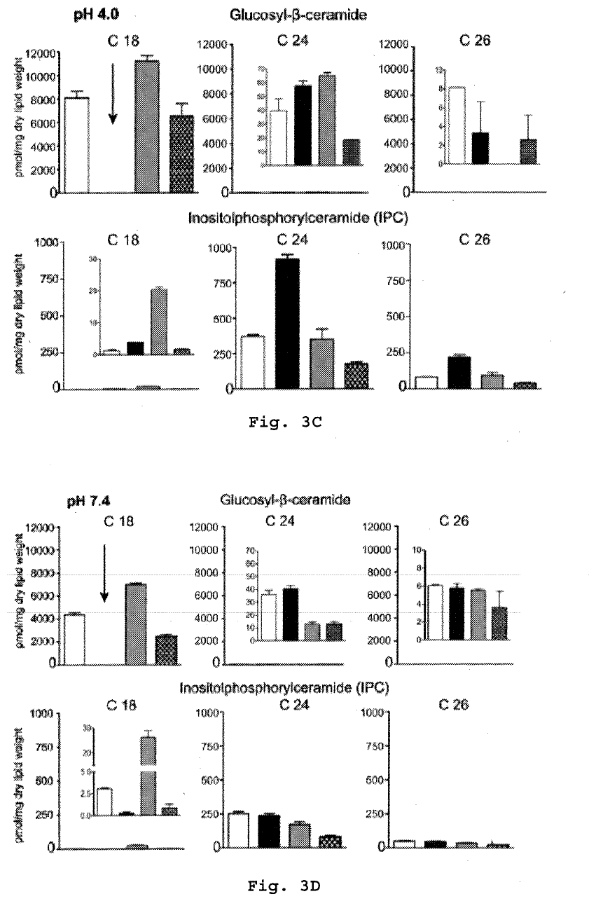

Tissue burden was assessed throughout the course of the experiment by removal of lungs and brain at days 0, 5, 10, 15 post infection. The number of Δcer1 cells in the lung decreases starting at day 5 and reduces to ˜3,500 cfu/lung at day 15, showing a decreased in lung CFU by 250-fold. At day 60, fungal cells were recovered in the lung (˜500 cfu) but only in 2 out of 10 mice, suggesting that most mice cleared the infection (FIG. 2C). In contrast to WT Cn infection, Δcer1 infected mice show no obvious signs of discomfort throughout the course of the experiment and are visually indistinguishable from uninfected mice. The Δcer1 cells were never observed to progress to the brain of infected mice (FIGS. 2D, 8A). In contrast, a significant number of cells were found in the lungs and brain of mice infected with WT and Δcer1+CER1 strains (FIGS. 3C and 3D). In both these control experiments, the number of cells in the brain increases over time, demonstrating a normal dissemination and subsequent onset of cryptococcosis. These observations are also confirmed by histopathology of the brain and lung, where no damage was observed to the lungs and brains of mice infected with Δcer1 (FIG. 2B). Mice infected with WT and Δcer1+CER1 showed significant lung and brain tissue damage (FIGS. 2B, 8A, 8B, 8C).

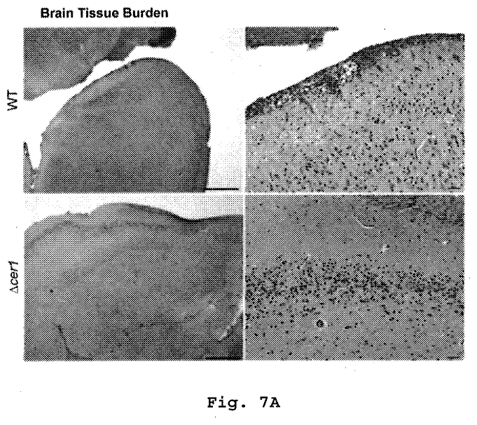

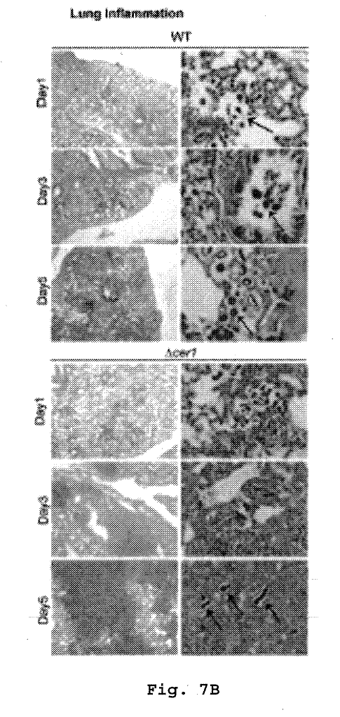

To understand the inflammatory response to Cn infection in the lungs, histopathology was performed at days 1, 3, 5 and day 60 (only Δcer1 mice survive to day 60). Lungs of Δcer1 infected mice show a high degree of immune cell infiltrate to the lung and alveolar spaces. As the experiment develops, progressive clearing of the immune cell infiltrate was observed (FIGS. 2B, 8B). Cells of Δcer1 can be seen in several areas of the lung until day 5. These cells appear to have difficulty completing replication and have a pseudohypal morphology. Lungs infected with WT show a persistently high level of immune cell infiltrate throughout the time course, enlarged Cn capsules, and fungal pneumonia, which is not observed for Δcer1. (FIG. 7B). The total burden of WT Cn cells increases throughout the time course (FIGS. 2A, 2B, 2C, 2D).

### Example 4. Identification of Lipid Changes on Ceramide Synthase Deletion

The sphingolipid pathway in Cn can be separated into 2 major branches: substrates that lead to the generation of glucose containing sphingolipids like glucosylceramide (GlcCer) or those that lead to inositol containing sphingolipids like inositol phosphorylceramide (IPC). To assess the specific roles of each gene in lipid metabolism, each knockout strain was analyzed with lipidomic focused mass spectrometry. The analysis was performed on cells grown in in vitro conditions (5% 002, 37° C., DMEM) mimicking host-like growth conditions.

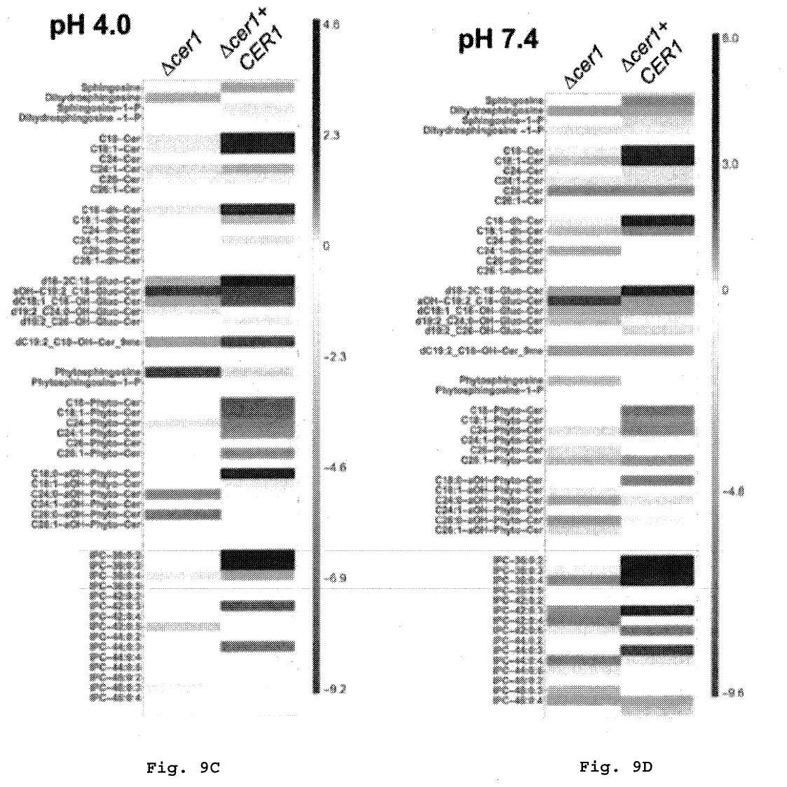

Since fungal cells produce a diverse array of ceramide species, mass spectrometry detection protocols were developed for an array of the most abundant ceramide lipids. When exposed to an acidic environment, WT Cn shows accumulation of dihydrosphingosine as well as higher total biomass of GlcCer species (FIGS. 3A, 3B, 3C, 3D, Table 2). We also observe higher abundance of phytosphingosine and certain long chain α-OH phytoceramides (C24, C26). This increase is propagated downstream in the pathway by increased levels of 42:0:4, 42:0:5 and 44:0:4 chain length IPCs (FIGS. 3A, 3B, 3C, 3D, Table 2). In contrast, at alkaline pH, sphingosine-1-phosphate, dihydrosphingosine-1-phosphate, and phytosphingosine-1-phosphate are twice as abundant as those in acidic pH (FIGS. 9A, 9B, 9C, 9D). Therefore, the data show that Cn not only changes the metabolism of several lipid species when it is exposed to different host conditions, but more importantly, this change depends on specific ceramide isoforms to generate sufficient amounts of the resulting complex sphingolipids. The lipid profile of Δcer1 is significantly perturbed from WT, as compared to the milder phenotypes of Δcer2 and Δcer3. We observed that Cer1 is the major ceramide synthase responsible for utilizing C18 fatty acyl CoA to generate C18 ceramides (FIG. 1B, 3A, 3B, 3C, 3D). As C18 ceramides are required to synthesize aOH-C19:2/C18 GlcCer, the most abundant GlcCer in either condition, the strain Δcer1 is lacking the vast majority of its normal glucose containing complex sphingolipids (FIG. 3C). C24 and C26 lipids are not significantly depleted in the Δcer1 strain, but also represent a tiny fraction of total GlcCer abundance in the WT strain (FIG. 3C). This observation agrees with the in vitro findings of chain length specificity for Cer1 (FIG. 1B). In the Δcer1 strain, depletion of IPCs generated from nonhydroxylated and α-hydroxylated C18 phytoceramides contrasts with the accumulation of long chain IPCs. Notably, Δcer1 and Δcer3 show a significant depletion of C26 phytoceramides in acidic conditions (FIG. 3A).

TABLE 2
Lipid species composition of SL biosynthetic pathway mutants.
Lipid abundance in WT, Δgcs1, GAL7::IPC1, Δcer1, Δcer2, and Δcer3
strains measured by LC-MS. Strains were grown in YNB, 2% glucose
before extracting lipids (see methods). C18 ceramide species include:
α-OH-Δ4-ceramide, α-OH-Δ4-Δ8 ceramide, and α-OH-Δ4-Δ8, 9Me
ceramide. All concentrations are represented as pmol/mg dry lipid weight.

| | | | GAL7:: | | | |
|---|---|---|---|---|---|---|
| Lipid species | Cn WT | Δgcs1 | IPC1 | Δcer1 | Δcer2 | Δcer3 |
| C18 Ceramide | 680.95 | 6037.44 | 1082.24 | 9.82 | 443.3 | 674.97 |
| species | | | | | | |
| α-OH-Δ4-Δ8, 9Me- | 3105.48 | 0.00 | 12823.82 | 4.96 | 1685.93 | 2821.22 |
| GlcCer | | | | | | |
| Phyto-Sph | 0.16 | 0.04 | 0.38 | 0.34 | 3.34 | 0.50 |
| Phyto-Sph-1P | 0.17 | 0.21 | 0.22 | 0.18 | 0.05 | 0.33 |
| PhytoC6 | 0.02 | 0.65 | 0.96 | 0.16 | 0.12 | 0.18 |
| PhytoC14-Cer | 0.18 | 0.25 | 0.85 | 0.69 | 4.16 | 1.20 |
| PhytoC16-Cer | 43.19 | 238.22 | 45.56 | 13.66 | 209.03 | 48.89 |
| PhytoC18:1-Cer | 178.33 | 190.04 | 120.55 | 9.51 | 98.66 | 188.16 |
| PhytoC18-Cer | 754.08 | 3245.00 | 121.75 | 17.37 | 569.30 | 541.11 |
| PhytoC20:1-Cer | 5.54 | 1.34 | 4.02 | 4.33 | 124.60 | 4.36 |
| PhytoC20-Cer | 8.84 | 11.69 | 2.95 | 5.37 | 94.65 | 5.25 |
| PhytoC22:1-Cer | 0.98 | 2.73 | 0.88 | 0.48 | 11.49 | 0.71 |
| PhytoC22-Cer | 44.25 | 10.79 | 17.04 | 21.34 | 67.46 | 12.64 |
| PhytoC24:1-Cer | 2.28 | 1.92 | 4.27 | 5.15 | 2.34 | 1.47 |
| PhytoC24-Cer | 583.66 | 136.33 | 260.58 | 667.00 | 742.17 | 350.59 |
| PhytoC26:1-Cer | 59.04 | 12.34 | 33.44 | 45.23 | 54.57 | 38.60 |
| PhytoC26-Cer | 86.78 | 34.83 | 41.82 | 119.93 | 284.50 | 57.28 |
| PhytoC28:1-Cer | 9.02 | 4.08 | 4.37 | 7.78 | 24.26 | 10.00 |
| PhytoC28-Cer | 4.78 | 5.32 | 6.03 | 3.58 | 19.37 | 3.93 |
| αOH-PhytoC14-Cer | 302.47 | 8.04 | 25.19 | 4.44 | 11.47 | 6.39 |
| αOH-PhytoC16-Cer | 9.61 | 0.00 | 0.00 | 0.00 | 0.00 | 0.00 |
| αOH-PhytoC18:1-Cer | 9.16 | 0.00 | 1.93 | 0.08 | 1.41 | 0.94 |
| αOH-PhytoC18-Cer | 111.05 | 98.39 | 185.51 | 65.06 | 623.64 | 90.02 |
| αOH-PhytoC20:1-Cer | 15.00 | 0.00 | 0.16 | 0.00 | 0.72 | 0.12 |
| αOH-PhytoC20-Cer | 29.65 | 30.15 | 31.78 | 20.74 | 42.00 | 19.00 |
| aOH-PhytoC22:1-Cer | 1.45 | 0.00 | 0.00 | 0.05 | 0.00 | 0.00 |
| αOH-PhytoC22-Cer | 67.94 | 91.14 | 65.80 | 62.26 | 92.91 | 42.93 |
| aOH-PhytoC24:1-Cer | 0.00 | 0.71 | 0.00 | 0.00 | 2.99 | 0.53 |
| αOH-PhytoC24-Cer | 2456.35 | 2217.29 | 4060.07 | 2796.37 | 1192.94 | 1391.92 |
| αOH-PhytoC26:1-Cer | 0.00 | 0.19 | 0.00 | 1.66 | 1.34 | 0.34 |
| aOH-PhytoC26-Cer | 125.07 | 115.01 | 266.64 | 348.00 | 165.48 | 90.21 |
| αOH-PhytoC28:1-Cer | 0.00 | 0.09 | 0.00 | 0.06 | 0.00 | 0.00 |
| αOH-PhytoC28-Cer | 0.00 | 0.03 | 0.00 | 0.02 | 0.00 | 0.00 |

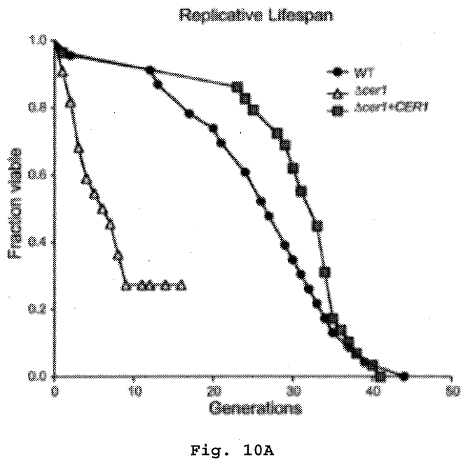

These data indicate that upon loss of Cer1, Cer1 and Cer3 can partially compensate for production of certain sphingolipid isoforms, but are insufficient for production of the major C18 ceramide isoforms. Broadly speaking, of the lipid isoforms showing significant change, these changes were most pronounced under acidic conditions (FIGS. 3A, 3B, 3C, 3D, 10A, 10B). The levels of the major IPC, IPC 42:0:4, remain relatively unchanged in the deletion strains. Δcer2 shows little to no depletion of lipids, but a marked increase in dihydrosphingosine, C18 dihydroceramide, and total short chain GlcCers. Additionally, Δcer2 shows a clear abundance of most C18 lipids in the IPC pathway: phytosphingosine, phytoceramides, αOH phytoceramides and C36 IPCs all were significantly more abundant as compared to WT. Meanwhile, Δcer3 shows a decrease in lipids along the GlcCer branch of the pathway as well as a decrease in C24 and C26 lipids in the IPC pathway under alkaline conditions. Together, these data suggest that Cer1 is critical for the biosynthesis of C18 ceramides, where Δcer2 and Δcer3 show more subtle lipid phenotypes, and appear to fine tune the abundance of less common lipids (FIGS. 9A, 9B, 9C, 9D).

### Example 4. Effect of Ceramide Synthase on the In Vitro Growth of C. neoformans

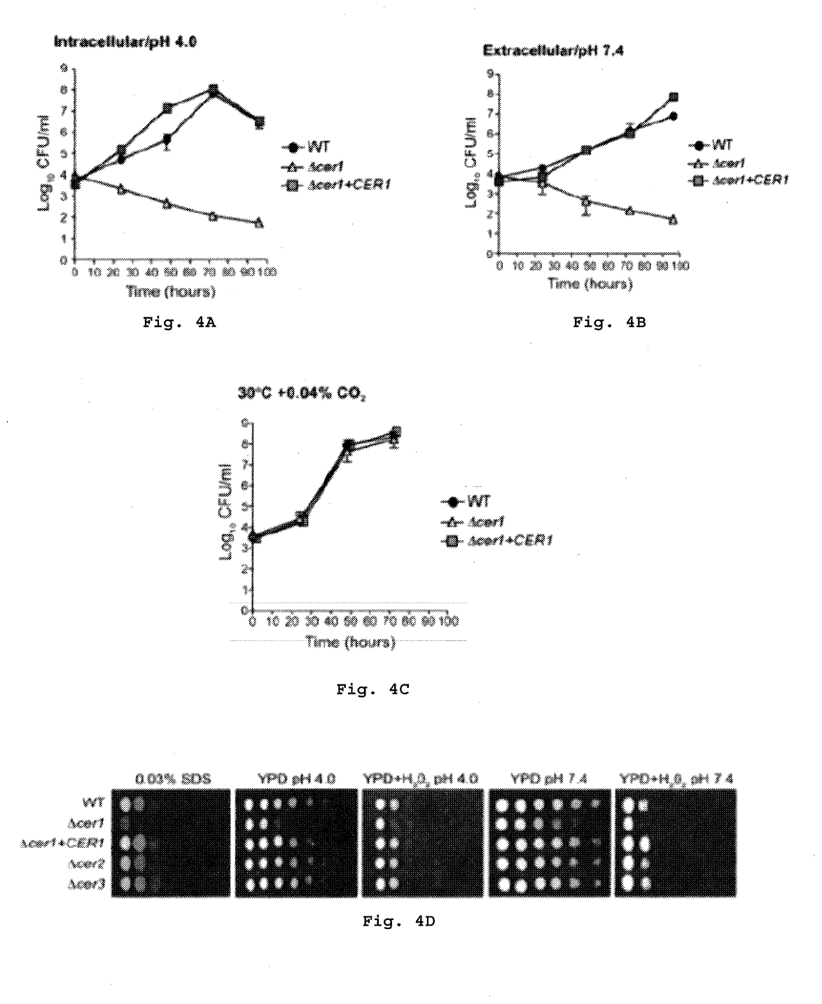

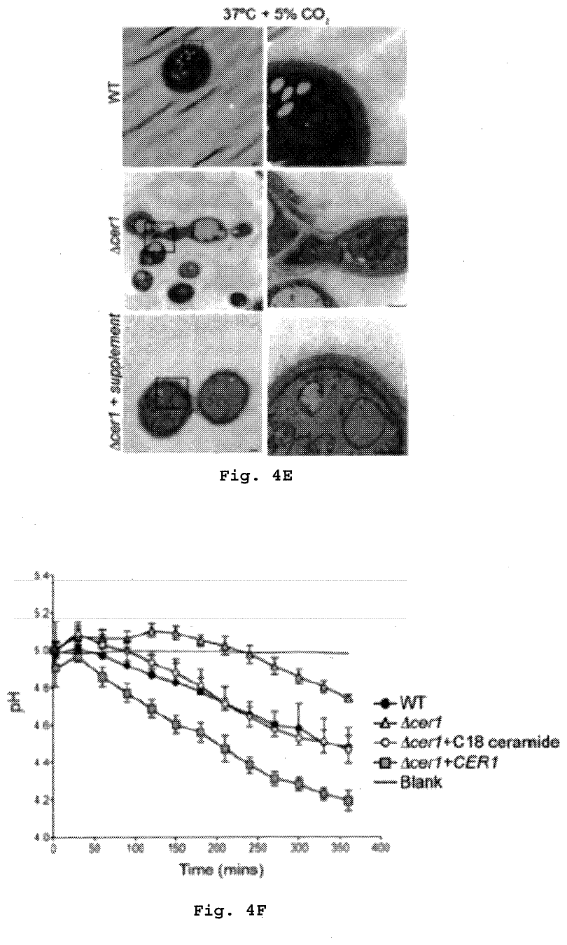

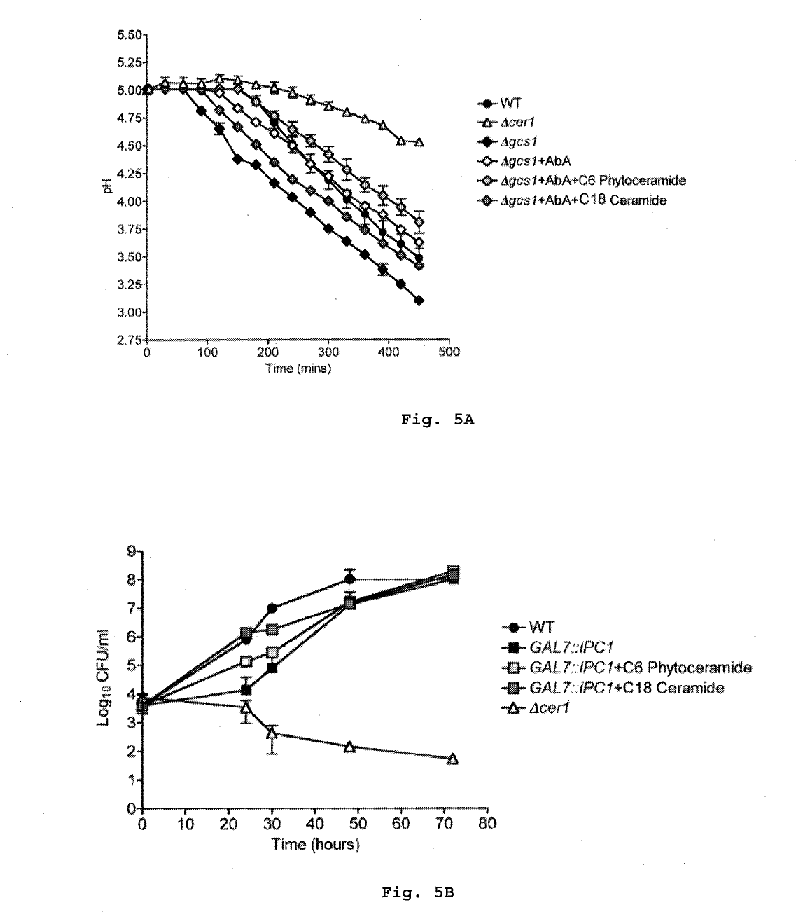

To evaluate the effect of gene deletion in In vitro cell culture growth, growth assays of Cn WT, Δcer1 and Δcer1+CER1 strains were performed. Growth was analyzed in conditions mimicking host intracellular and extracellular conditions, using DMEM at neutral/alkaline or acidic conditions at 37° C. and 5% CO₂. Δcer1 cells showed a distinct lack of growth at both conditions mimicking host environments (FIGS. 4A, 4B). Δcer1 viability began to decrease at 24 hours in acidic as well as alkaline conditions. Loss of viability was not observed at 30° C. and atmospheric level of CO₂ (FIG. 4C). These results indicate the deletion of Δcer1 is important for the survival and proliferation of Cn in host intracellular and extracellular conditions.

To determine the effect of cell wall stress on the deletion strains, a spot assay was performed by exposing WT, Δcer1, and Δcer1+CER1 to 0.03% SDS. It was observed that Δcer1 was more sensitive to cell wall stress as compared to WT and Δcer1+CER1 (FIG. 4D). The resilience of these strains to oxidative stress was tested by exposing dilutions of these cells to hydrogen peroxide at pH 4 and 7.4 (FIG. 4D). Δcer1 was hypersensitive to oxidative stress at both pH values. The hypersensitivity of Δcer1 to low pH and other stresses are significant considering that the spot assay was done using rich medium (YPD), as it could not be performed in minimum media for the growing defect phenotype of the mutant.

### Example 5. Phenotype Analysis of Ceramide Synthase Mutant Strains

To assess the physiological effects of the deletion of ceramide synthase genes, the phenotypes of these cells were analyzed under several conditions with a focus on alteration of virulence factors. The deletion of Cer1, but not Cer1 or Cer3, generated defects in cell morphology. Capsule visualization by India ink staining revealed that cells of Δcer1 had cell division defects leading to development of elongated cells with a smaller capsule (FIG. 10B). When Δcer1 was grown in rich media (YPD) the cells show normal morphology, with a few cells showing multiple enlarged buds that remained attached. However, upon transfer to host-like growth conditions (5% CO₂, 37° C., DMEM), Δcer1 cells show gross morphological defects and an inability to complete cytokinesis. A lifespan study of these cells showed that the average replicative lifespan (RLS) of Δcer1 was only 6.5 generations while that of WT and Δcer1+CER1 was 27 and 30 generations, respectively (FIG. 10A). For Δcer1, after 6.5±2 generations, the cells formed elongated pseudohyphal-like structures where new buds were impossible to separate from the mother cell. Despite the inability to separate, the cells continued to elongate for several hours.

Transmission Electron Microscopy images were obtained to further observe these changes in cellular structure. Δcer1 cells show detachment of the cell wall from the plasma membrane in many cells. The elongated “hyphal-like” structures observed in histological and India ink staining were found to be daughter cells that were unable to complete cytokinesis. Interestingly, it was also observed that the cell wall structure of Δcer1 looked very different from that of WT. While the WT cells show well defined, distinct layers resulting in a compact cell wall, the cell wall of Δcer1 appeared less compact, with no clear separation between cell wall layers (FIG. 4E). The fibrilar structures forming the polysaccharide capsule were smaller and less distinct than that of WT (FIG. 4E). It is hypothesized that the layers of the cell wall of Δcer1 are inhibitory to cytokinesis and cell separation under two conditions: when the cells are exposed to host-like conditions, or when cells are grown in the absence of rich, ceramide lipid containing media. To confirm the role of ceramides in the formation of such a drastic cellular defect, the cells of Δcer1 were supplemented with a cocktail of natural ceramides (Matreya LLC). Upon supplementation, it was observed that many of the previously observed cellular defects were recovered (FIG. 4E). This cocktail predominantly contains a mixture of C18 and C24 hydroxylated and nonhydroxylated ceramides. Together, these observations suggest that ceramides synthesized by Cer1 are critical for proper cytokinesis in stressful host conditions, where increased cell wall thickness introduces a hindrance for proper daughter cell budding.

### Example 6. C18 Ceramides Generated by Cer1 are Important for Acidic Tolerance of C. neoformans

The efficiency of the plasma membrane proton pump, Pma1, was checked upon deletion of a ceramide synthase in Cn. Pma1 is a crucial mediator of Cn virulence, as the proton pump regulates Cn cytosolic pH. This is a particularly important function for Cn growth inside acidic macrophage lysosomes. A colorimetric pH indicating dye was used to measure glucosedependent proton efflux over a time course (Soteropoulos et al., 2000). The results showed Δcer1 cells acidify medium at a much slower rate than WT and Δcer1+CER1 strains (FIG. 4F). An increase in Pma1 efficiency is shown in Δcer1 cells upon supplementation with C18 ceramide (FIG. 4F).

To further confirm to the role of ceramides in the observed phenotypes, Pma1 activity of Δgcs1 strain was tested, as well as the Δgcs1 strain treated with an IPC1 inhibitory drug, Aureobaisdin A (AbA). It was observed that Δgcs1 has high Pma1 activity, which is reduced upon treatment with AbA (FIG. 5A). Interestingly, supplementation with C18 ceramides, but not phytoceramides, increases Pma1 activity of AbA treated Δgcs1 cells (FIG. 5A). To ensure the Pma1 activity data was not attributable to higher or lower rates of cell death, cell viability was measured at the end of each experiment, and no differences were found between strains. To further clarify the role of intermediate ceramides, a genetically down-regulated IPC1 strain (GAL7::IPC1) was used, which has been reported to show reduced growth in acidic conditions. The growth defect of this strain was compensated by supplementation C18 ceramide, and to a smaller extent by C6 phytoceramide (FIG. 5B, Table 3). These data indicate the intermediate ceramide isoforms, not the terminal IPC and GlcCer isoforms, play a major role in regulation of Pma1 and normal cell growth.

TABLE 3
C18 ceramide abundance of GAL7::IPC1 at 6 hours, 12 hours, 24 hours
and 48 hours post glucose inoculation. C18 ceramide changes of
WT and GAL7::IPC1 upon glucose transfer. When Ipc1 is genetically
downregulated, a transient decrease is observed in C18 ceramide
levels because cells do not tolerate high levels of ceramides.
As the cells start to adapt, growth is restored likely due to
a parallel increase in C18 ceramides. These C18 ceramides then
lead to an increase in GlcCer which is highly abundant in GAL7::IPC1
at 48 hours post glucose inoculation (Table 2).

| | | GAL7::IPC1 | | GAL7::IPC1 |
|---|---|---|---|---|
| Time | WT pH 4.0 | pH 4.0 | WT pH 7.0 | pH 7.0 |
| 6 hours | 23.93 | 10.90 | 18.64 | 6.49 |
| 12 hours | 31.80 | 3.152 | 30.46 | 0.043 |
| 24 hours | 11.46 | 37.534 | 18.41 | 6.90 |
| 48 hours | 20.53 | 34.64 | 25.473 | 8.59 |

### Example 7. Assessing Biochemical Enzymatic Activity of Cn Cer1 Enzyme

An assay was set up to directly assess the biochemical enzymatic activity of the Cn Cer1 enzyme. To do so, the Cn Cer1 cDNA was tagged with a V5 and HIS tags and expressed in the model yeast Saccharomyces cerevisiae (Sc). Upon microsomal preparation and partial affinity purification, the Cn Cer1 was assayed for activity using an assay adapted from the one used for human ceramide synthases. It was discovered that the protein is active when expressed in Sc and its activity is comparable to the human Cer1 (Hu Cer1) using Thin Layer Chromatography (TLC) (FIG. 11). Thus, using this assay, compounds were screened that would target the fungal Cer1 but not the mammalian Cer enzymes. This was achieved by adapting the ceramide synthase assay to a 96-well plate format using the Ceramide Synthase Assay Kit available from Avanti Polar Lipids.

The Z′ factor of the assay was then obtained to make sure it could differentiate positive versus negative hits (FIG. 12).

### Example 8. High Throughout Assay Compound Screening—Compounds 1-10

An initial screening yielded compounds 1-10 (Table 3). The compounds were diluted to 1 mM (1:10 dilution with 10% DMSO) with a physiological buffer (YNB medium buffered with HEPES at pH 7.4 containing 2% glucose) and subsequently diluted to 300 μM (1:3.3 dilution) with the same medium (3% DMSO). A 100-μl aliquot of this solution was placed into a well of a 96-well master plate. The ceramide synthase reaction was performed in a 96-well plate format using fungal Cer1 (Cn Cer1) containing NBD-Sphingosine (NBD-Sph) and 18:0 Coenzyme A as substrates using the following concentrations: 10 μM NBD-Sph and 50 μM fatty acid CoA. The reaction mixture was prepared in HEPES buffer (20 mM HEPES, pH 7.2, 25 mM KCl, 250 mM sucrose, and 2 mM MgCl₂). The library compounds were added and plate incubated for 1 hour at 37° C. The lipid product (NBD-ceramide) was separated using solid phase extraction (SPE) column chromatography using Strata® C18-E, 96 well plates and the reaction product was measured using a plate reader. Compounds 1-10 were found to inhibit Cn Cer1 synthase activity at 1 micromolar concentration by 94.3% with a Z′factor of 0.531175.

### Example 9. High Throughout Assay Compound Screening—Compound 11-20

An initial screening yielded compounds 11-20 (Table 3). The compounds were diluted to 1 mM (1:10 dilution with 10% DMSO) with a physiological buffer (YNB medium buffered with HEPES at pH 7.4 containing 2% glucose) and subsequently diluted to 300 μM (1:3.3 dilution) with the same medium (3% DMSO). A 100-μl aliquot of this solution was placed into a well of a 96-well master plate. The ceramide synthase reaction was performed in a 96-well plate format using fungal Cer1 (Cn Cer1) containing NBD-Sphingosine (NBD-Sph) and 18:0 Coenzyme A as substrates using the following concentrations: 10 μM NBD-Sph and 50 μM fatty acid CoA. The reaction mixture was prepared in HEPES buffer (20 mM HEPES, pH 7.2, 25 mM KCl, 250 mM sucrose, and 2 mM MgCl₂). The library compounds were added and plate incubated for 1 hour at 37° C. The lipid product (NBD-ceramide) was separated using solid phase extraction (SPE) column chromatography using Strata® C18-E, 96 well plates and the reaction product was measured using a plate reader. Compounds 1-10 were found to inhibit Cn Cer1 synthase activity at 1 micromolar concentration by 87.7% with a Z′factor of 0.623352.

### Example 10. High Throughout Assay Compound Screening—Compounds 21-30

An initial screening yielded compounds 21-30 (Table 3). The compounds were diluted to 1 mM (1:10 dilution with 10% DMSO) with a physiological buffer (YNB medium buffered with HEPES at pH 7.4 containing 2% glucose) and subsequently diluted to 300 μM (1:3.3 dilution) with the same medium (3% DMSO). A 100-μl aliquot of this solution was placed into a well of a 96-well master plate. The ceramide synthase reaction was performed in a 96-well plate format using fungal Cer1 (Cn Cer1) containing NBD-Sphingosine (NBD-Sph) and 18:0 Coenzyme A as substrates using the following concentrations: 10 μM NBD-Sph and 50 μM fatty acid CoA. The reaction mixture was prepared in HEPES buffer (20 mM HEPES, pH 7.2, 25 mM KCl, 250 mM sucrose, and 2 mM MgCl₂). The library compounds were added and plate incubated for 1 hour at 37° C. The lipid product (NBD-ceramide) was separated using solid phase extraction (SPE) column chromatography using Strata® C18-E, 96 well plates and the reaction product was measured using a plate reader. Compounds 1-10 were found to inhibit Cn Cer1 synthase activity at 1 micromolar concentration by 86.09% with a Z′factor of 0.531175.

### Example 11. High Throughout Assay Compound Screening—Compounds 31-40

An initial screening yielded compounds 31-40 (Table 3). The compounds were diluted to 1 mM (1:10 dilution with 10% DMSO) with a physiological buffer (YNB medium buffered with HEPES at pH 7.4 containing 2% glucose) and subsequently diluted to 300 μM (1:3.3 dilution) with the same medium (3% DMSO). A 100-μl aliquot of this solution was placed into a well of a 96-well master plate. The ceramide synthase reaction was performed in a 96-well plate format using fungal Cer1 (Cn Cer1) containing NBD-Sphingosine (NBD-Sph) and 18:0 Coenzyme A as substrates using the following concentrations: 10 μM NBD-Sph and 50 μM fatty acid CoA. The reaction mixture was prepared in HEPES buffer (20 mM HEPES, pH 7.2, 25 mM KCl, 250 mM sucrose, and 2 mM MgCl₂). The library compounds were added and plate incubated for 1 hour at 37° C. The lipid product (NBD-ceramide) was separated using solid phase extraction (SPE) column chromatography using Strata® C18-E, 96 well plates and the reaction product was measured using a plate reader. Compounds 1-10 were found to inhibit Cn Cer1 synthase activity at 1 micromolar concentration by 85.7% with a Z′factor of 0.623352.

### Example 12. High Throughout Assay Compound Screening—Compounds 41-50

An initial screening yielded compounds 21-30 (Table 3). The compounds were diluted to 1 mM (1:10 dilution with 10% DMSO) with a physiological buffer (YNB medium buffered with HEPES at pH 7.4 containing 2% glucose) and subsequently diluted to 300 μM (1:3.3 dilution) with the same medium (3% DMSO). A 100-μl aliquot of this solution was placed into a well of a 96-well master plate. The ceramide synthase reaction was performed in a 96-well plate format using fungal Cer1 (Cn Cer1) containing NBD-Sphingosine (NBD-Sph) and 18:0 Coenzyme A as substrates using the following concentrations: 10 μM NBD-Sph and 50 μM fatty acid CoA. The reaction mixture was prepared in HEPES buffer (20 mM HEPES, pH 7.2, 25 mM KCl, 250 mM sucrose, and 2 mM MgCl₂). The library compounds were added and plate incubated for 1 hour at 37° C. The lipid product (NBD-ceramide) was separated using solid phase extraction (SPE) column chromatography using Strata® C18-E, 96 well plates and the reaction product was measured using a plate reader. Compounds 1-10 were found to inhibit Cn Cer1 synthase activity at 1 micromolar concentration by 84.9% with a Z′factor of 0.623352.

TABLE 4
Screened compounds obtained from high throughput assay; inhibition
of Cn Cer1 synthse activity

| | ChemBridge | | % reduction |
|---|---|---|---|
| Compound | ID # | Chemical Structure | in activity |
| 1 | 19364456 | 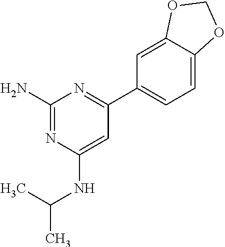 [SMILES: CC(C)Nc1cc(-c2ccc3c(c2)OCO3)nc(N)n1] | 94.43169782 |
| 2 | 41542111 | 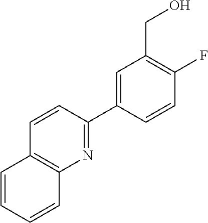 [SMILES: OCc1cc(-c2ccc3ccccc3n2)ccc1F] | 94.43169782 |
| 3 | 53160590 | 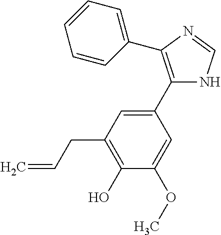 [SMILES: C=CCc1cc(-c2[nH]cnc2-c2ccccc2)cc(OC)c1O] | 94.43169782 |
| 4 | 24395225 | 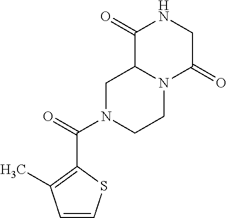 [SMILES: Cc1ccsc1C(=O)N1CCN2C(=O)CNC(=O)C2C1] | 94.43169782 |
| 5 | 25751303 | 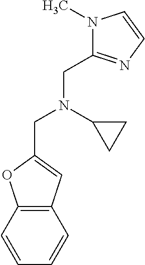 [SMILES: Cn1ccnc1CN(Cc1cc2ccccc2o1)C1CC1] | 94.43169782 |
| 6 | 63238324 | 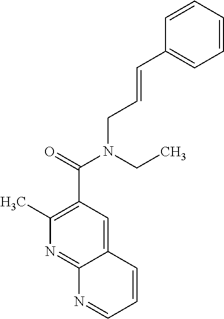 [SMILES: CCN(C/C=C/c1ccccc1)C(=O)c1cc2cccnc2nc1C] | 94.43169782 |
| 7 | 59580690 | 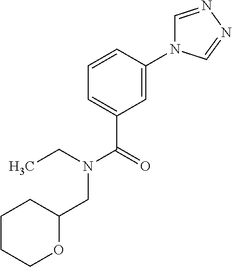 [SMILES: CCN(CC1CCCCO1)C(=O)c1cccc(-n2cnnc2)c1] | 94.43169782 |
| 8 | 81830236 | 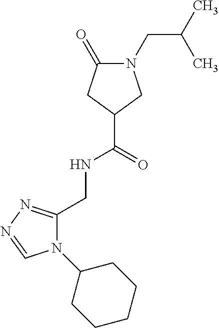 [SMILES: CC(C)CN1CC(C(=O)NCc2nncn2C2CCCCC2)CC1=O] | 94.43169782 |
| 9 | 79305044 | 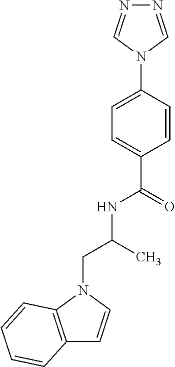 [SMILES: CC(Cn1ccc2ccccc21)NC(=O)c1ccc(-n2cnnc2)cc1] | 94.43169782 |
| 10 | 11115550 | 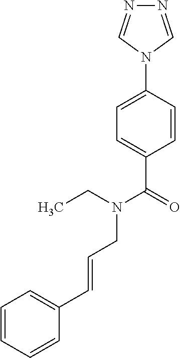 [SMILES: CCN(C/C=C/c1ccccc1)C(=O)c1ccc(-n2cnnc2)cc1] | 94.43169782 |
| 11 | 35339737 | 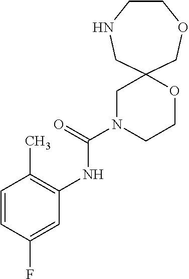 [SMILES: Cc1ccc(F)cc1NC(=O)N1CCOC2(CNCCOC2)C1] | 87.67241822 |
| 12 | 35775621 | 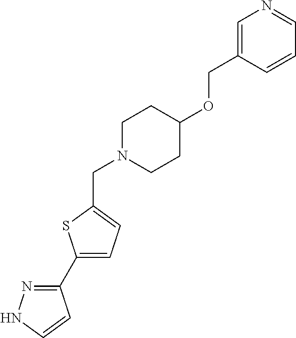 [SMILES: c1cncc(COC2CCN(Cc3ccc(-c4cc[nH]n4)s3)CC2)c1] | 87.67241822 |
| 13 | 99306308 | 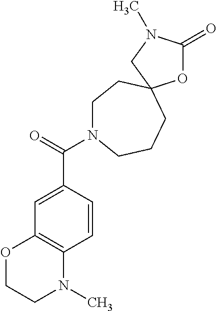 [SMILES: CN1CC2(CCCN(C(=O)c3ccc4c(c3)OCCN4C)CC2)OC1=O] | 87.67241822 |
| 14 | 47718645 | 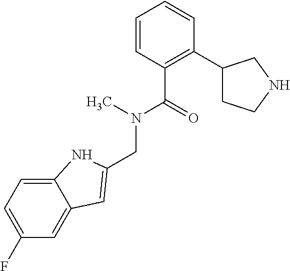 [SMILES: CN(Cc1cc2cc(F)ccc2[nH]1)C(=O)c1ccccc1C1CCNC1] | 87.67241822 |
| 15 | 25890190 | 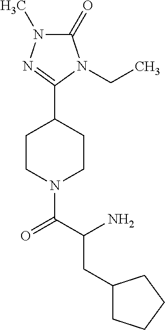 [SMILES: CCn1c(C2CCN(C(=O)C(N)CC3CCCC3)CC2)nn(C)c1=O] | 87.67241822 |
| 16 | 12410439 | 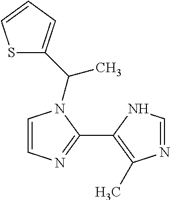 [SMILES: Cc1nc[nH]c1-c1nccn1C(C)c1cccs1] | 87.67241822 |
| 17 | 50766837 | 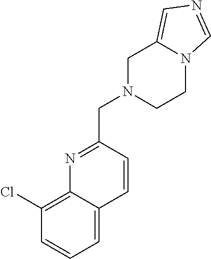 [SMILES: Clc1cccc2ccc(CN3CCn4cncc4C3)nc12] | 87.67241822 |
| 18 | 25614755 | 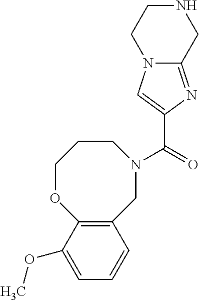 [SMILES: COc1cccc2c1OCCCN(C(=O)c1cn3c(n1)CNCC3)C2] | 87.67241822 |
| 19 | 92164704 | 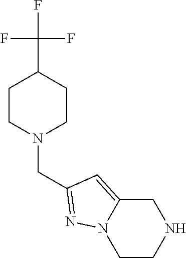 [SMILES: FC(F)(F)C1CCN(Cc2cc3n(n2)CCNC3)CC1] | 87.67241822 |
| 20 | 55093716 | 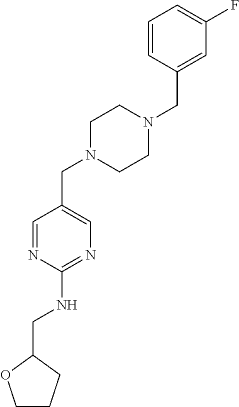 [SMILES: Fc1cccc(CN2CCN(Cc3cnc(NCC4CCCO4)nc3)CC2)c1] | 87.67241822 |
| 21 | 21920728 | 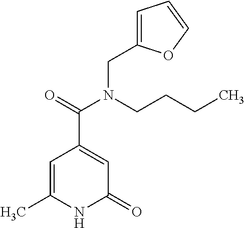 [SMILES: CCCCN(Cc1ccco1)C(=O)c1cc(C)[nH]c(=O)c1] | 86.09789425 |
| 22 | 25343277 | 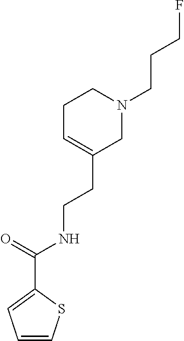 [SMILES: O=C(NCCC1=CCCN(CCCF)C1)c1cccs1] | 86.09789425 |
| 23 | 56009300 | 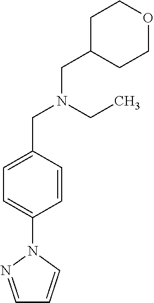 [SMILES: CCN(Cc1ccc(-n2cccn2)cc1)CC1CCOCC1] | 86.09789425 |
| 24 | 10656689 | 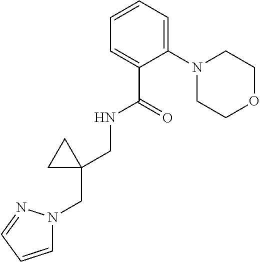 [SMILES: O=C(NCC1(Cn2cccn2)CC1)c1ccccc1N1CCOCC1] | 86.09789425 |
| 25 | 85599533 | 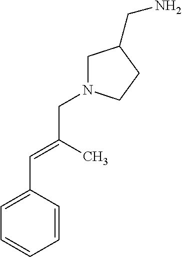 [SMILES: C/C(=C\c1ccccc1)CN1CCC(CN)C1] | 86.09789425 |
| 26 | 87690891 | 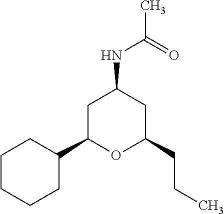 [SMILES: CCC[C@@H]1C[C@H](NC(C)=O)C[C@H](C2CCCCC2)O1] | 86.09789425 |
| 27 | 37567033 | 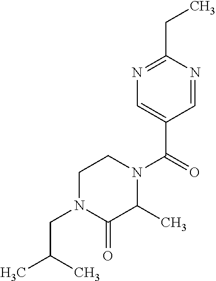 [SMILES: CCc1ncc(C(=O)N2CCN(CC(C)C)C(=O)C2C)cn1] | 86.09789425 |
| 28 | 85236661 | 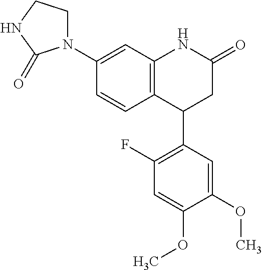 [SMILES: COc1cc(F)c(C2CC(=O)Nc3cc(N4CCNC4=O)ccc32)cc1OC] | 86.09789425 |
| 29 | 66896553 | 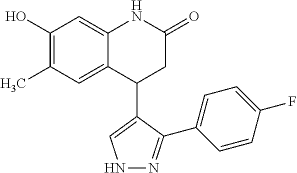 [SMILES: Cc1cc2c(cc1O)NC(=O)CC2c1c[nH]nc1-c1ccc(F)cc1] | 86.09789425 |
| 30 | 43797164 | 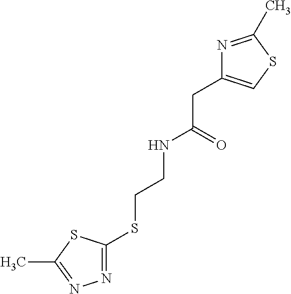 [SMILES: Cc1nc(CC(=O)NCCSc2nnc(C)s2)cs1] | 86.09789425 |
| 31 | 83646679 | 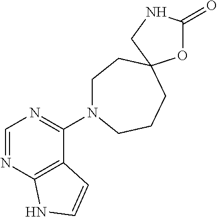 [SMILES: O=C1NCC2(CCCN(c3ncnc4[nH]ccc34)CC2)O1] | 85.72918984 |
| 32 | 16814203 | 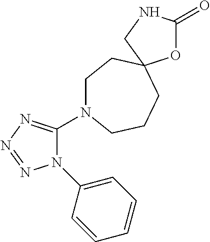 [SMILES: O=C1NCC2(CCCN(c3nnnn3-c3ccccc3)CC2)O1] | 85.72918984 |
| 33 | 93171679 | 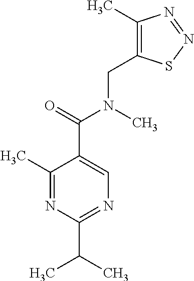 [SMILES: Cc1nc(C(C)C)ncc1C(=O)N(C)Cc1snnc1C] | 85.72918984 |
| 34 | 29695096 | 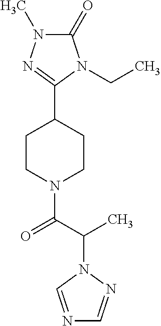 [SMILES: CCn1c(C2CCN(C(=O)C(C)n3cncn3)CC2)nn(C)c1=O] | 85.72918984 |
| 35 | 25132320 | 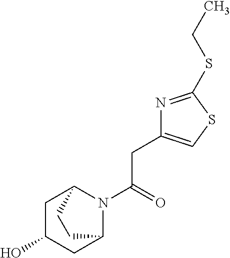 [SMILES: CCSc1nc(CC(=O)N2[C@@H]3CC[C@H]2C[C@@H](O)C3)cs1] | 85.72918984 |
| 36 | 21144928 | 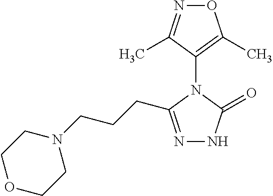 [SMILES: Cc1noc(C)c1-n1c(CCCN2CCOCC2)n[nH]c1=O] | 85.72918984 |
| 37 | 60452825 | 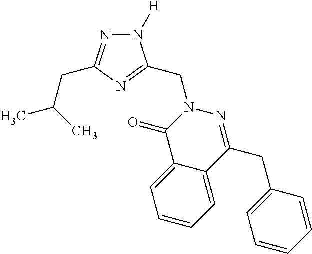 [SMILES: CC(C)Cc1n[nH]c(Cn2nc(Cc3ccccc3)c3ccccc3c2=O)n1] | 85.72918984 |
| 38 | 33176385 | 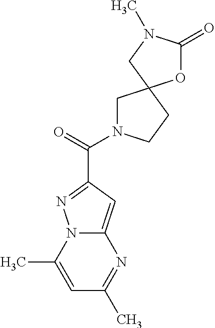 [SMILES: Cc1cc(C)n2nc(C(=O)N3CCC4(CN(C)C(=O)O4)C3)cc2n1] | 85.72918984 |
| 39 | 79482532 | 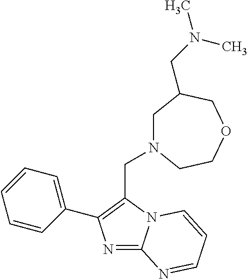 [SMILES: CN(C)CC1COCCN(Cc2c(-c3ccccc3)nc3ncccn23)C1] | 85.72918984 |
| 40 | 41792359 | 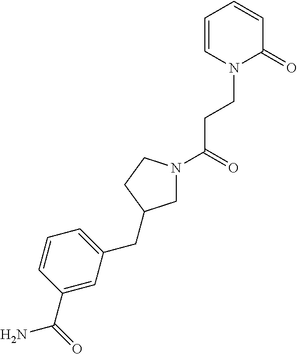 [SMILES: NC(=O)c1cccc(CC2CCN(C(=O)CCn3ccccc3=O)C2)c1] | 85.72918984 |
| 41 | 96925858 | 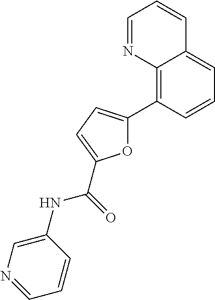 [SMILES: O=C(Nc1cccnc1)c1ccc(-c2cccc3cccnc23)o1] | 84.91436012 |
| 42 | 41066993 | 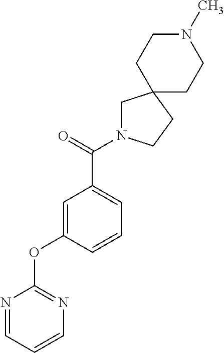 [SMILES: CN1CCC2(CC1)CCN(C(=O)c1cccc(Oc3ncccn3)c1)C2] | 84.91436012 |
| 43 | 26270082 | 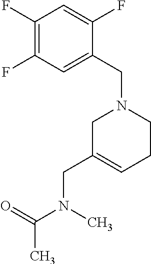 [SMILES: CC(=O)N(C)CC1=CCCN(Cc2cc(F)c(F)cc2F)C1] | 84.91436012 |
| 44 | 48873705 | 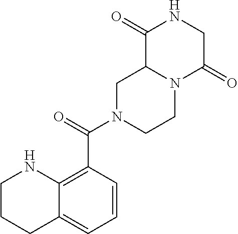 [SMILES: O=C1NCC(=O)N2CCN(C(=O)c3cccc4c3NCCC4)CC12] | 84.91436012 |
| 45 | 31004445 | 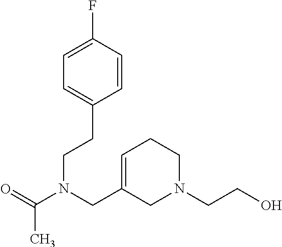 [SMILES: CC(=O)N(CCc1ccc(F)cc1)CC1=CCCN(CCO)C1] | 84.91436012 |
| 46 | 57204986 | 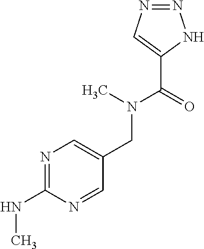 [SMILES: CNc1ncc(CN(C)C(=O)c2cnn[nH]2)cn1] | 84.91436012 |
| 47 | 55298305 | 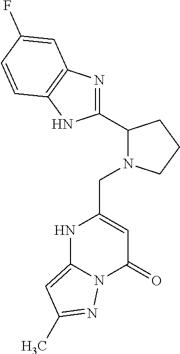 [SMILES: Cc1cc2[nH]c(CN3CCCC3c3nc4cc(F)ccc4[nH]3)cc(=O)n2n1] | 84.91436012 |
| 48 | 92518520 | 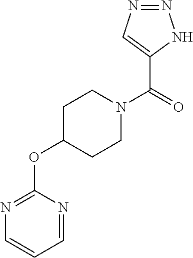 [SMILES: O=C(c1cnn[nH]1)N1CCC(Oc2ncccn2)CC1] | 84.91436012 |
| 49 | 26693087 | 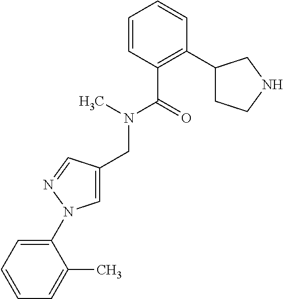 [SMILES: Cc1ccccc1-n1cc(CN(C)C(=O)c2ccccc2C2CCNC2)cn1] | 84.91436012 |
| 50 | 88891834 | 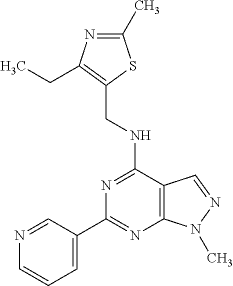 [SMILES: CCc1nc(C)sc1CNc1nc(-c2cccnc2)nc2c1cnn2C] | 84.91436012 |

### Example 13. Administration of the Compound

An amount of the compound of the present invention is administered to a subject afflicted with a fungal infection. The amount of the compound is effective to treat the subject.

An amount of the compound of the present invention is administered to a subject afflicted with a fungal infection. The amount of the compound is effective to treat the subject by inhibiting fungal Cer1 activity in the fungus without substantially inhibiting human ceramide synthase activity in the subject.

An amount of the compound of the present invention in combination with an anti-fungal agent are administered to a subject afflicted with a fungal infection. The amount of the compound and the agent are effective to treat the subject. In some embodiments, the anti-fungal agent is amphotericin B, fluconazole, itraconazole, voriconazole, and other azoles, caspofungin and other echinocandins or terbinafine.

Compounds 1-50 are inhibitors of fungal Cer1. Additional inhibitors within the scope of this invention also inhibit fungal Cer1. The compounds of the present are advantageous in that they do not inhibit human ceramide synthase activity. Australifungin is a known Cer1 inhibitor. However, Australifungin targets human Cer1.

### Discussion

An understanding of the mechanisms driving Cn pathogenicity is an increasingly important area of research due to the growing number of cases of cryptococcosis worldwide. A deeper insight into how this pathogen efficiently maintains itself within immunocompromised hosts will bring scientists closer to finding a way to control its growth and dissemination. Dynamic adjustment of the sphingolipid profile in Cn cells has been reported to play a significant role in Cn's ability to grow in either highly acidified phagocytic environment or slightly alkaline blood, alveolar, and brain tissue environments. The present study shows how ceramide synthase Cer1 is a major factor in the pathogenicity of Cn. It was observed that deletion of each ceramide synthase shows a survival curve that was significantly divergent from WT infection, with Δcer1 being completely avirulent with 80% of mice totally clearing the infection within 60 days. Lack of in vitro growth in host intracellular and extracellular conditions suggest Δcer1 lacks the ability to efficiently thrive in the host upon infection. In stark contrast to previous studies on Δgcs1 and GAL7::IPC1, cells of Δcer1 quickly begin to die when grown in minimal medium conditions regardless of either pH. The results show that Δcer1 has a marked reduction in the activity of Pma1 thereby suggesting a role, possibly through an indirect mechanism, of C18 ceramides in the ability of Cn to maintain its survival within the highly acidic phagolysosome. Increased Pma1 activity in the Δgcs1 mutant suggests two possible models of ceramide species mediated regulation of Pma1: it is possible the lack of GlcCer species in Δgcs1 relieves an inhibitory pressure on Pma1 activity, causing the observed increase. Alternatively, a buildup of intermediate ceramide compounds or IPCs in response to a lack of Goal (Table 2) could have a positive effect on Pma1 activity, which could also result in the observed increase. Pma1 activity in the presence of AbA and Δgcs1 indicates intermediate ceramide compounds play a major role in the enzyme's activity.

Δcer1 cells have critical defects in cytokinesis in the presence of host-like environmental stresses. The cell wall is improperly anchored to the plasma membrane in many cells, consequently the membrane and cell wall structure may be inhibiting daughter cell separation. It remains unclear if the altered morphology of the cell wall or the lack of cell wall adherence to the plasma membrane is the major contributor to this phenotype. The presence of irregular amounts of long chain ceramides and complex sphingolipids could be affecting the rigidity of the cell wall and/or plasma membrane. It was observed that each knockout strain has distinct alterations of ceramides and downstream lipids as shown by lipidomic analysis, indicating the genes are not functionally redundant, and each likely plays a specific role in maintaining the optimum lipid profile for Cn membranes. Furthermore, the inability of Δcer1 cells to survive in acidic conditions, despite abundant amounts of IPC-42:0:4, strongly suggests an important role of C18 ceramides for this phenotype.

Because of the striking phenotype of the mutant Δcer1, it is believed that targeting this enzymatic activity will be effective in killing fungal cells, as Ca, Af and many other fungi also possess the Cer1 homolog. Further, since Cn Cer1 is conserved in many fungi and different than human CerS enzymes, the discovery and characterization of Cn CerS1 has opened multiple gates for anti-fungal drug discovery.

In the instant invention, methods of inhibiting fungal ceramide synthase using compounds that target Cn CerS1 to treat Cryptococcus neoformans (Cn) infection are disclosed. A solid phase 96 well plate was used for screening drug libraries to identify compounds that inhibit fungal Cer1 (i.e., the fungal ceramide synthase enzyme delta-6717), but not the mammalian Cer enzymes. As previously disclosed, Cryptococcus neoformans delta-6717 cannot grow in conditions mimicking mammalian environments and is therefore not pathogenic in a mouse model of cryptococcal meningitis. The delta-6717 mutant does not produce certain ceramide species that are part of the synthesis of glucosylceramide, and thus, delta-6717 does not produce glucosylceramide. The fungal ceramide synthase enzymes do not include human ceramide synthases CerS1/CerS4, CerS2, CerS3, CerS5, or CerS6.

Preliminary data using primary in-vitro assay for informed drug design/medicinal chemistry have identified several compounds capable of inhibiting Cn Cer 1 synthase activity. The studies presented here are novel and provide new insights regarding the antifungal therapeutic efficacy of this chemotype and mechanism.

In summary, this study is a novel insight into the critical importance of ceramides and ceramide synthases in cryptococcal pathogenicity. Cer1 predominately utilizes C18 fatty acid chain substrates in order to synthesize specific ceramides and complex sphingolipids that are of crucial importance towards Cn pathogenicity. The results can be extrapolated to several other pathogenic fungi, and therefore provide hope for developing new antifungal agents to help immunocompromised individuals susceptible to fungal infections.

### REFERENCES

Aguilera-Romero, A., Gehin, c. & Riezman, H. 2014. Sphingolipid homeostasis in the web of metabolic routes. Biochim Biophys Acta, 1841, 647-56.
Alvarez, M. & Casadevall, A. 2006. Phagosome extrusion and host-cell survival after Cryptococcus neoformans phagocytosis by macrophages. Curr Biol, 16, 2161-5.
Bligh, E. G. & Dyer, W. J. 1959. A rapid method of total lipid extraction and purification. Can J Biochem Physiol, 37, 911-7.
Cheon, S. A., Bal, J., Song, Y., Hwang, H. M., Kim, A. R., Kang, W. K., Kang, H. A., Hannibal-Bach, H. K., Knudsen, J., Ejsing, C. S. & Kim, J. Y. 2012. Distinct roles of two ceramide synthases, CaLag1p and CaLac1p, in the morphogenesis of Candida albicans. Molecular microbiology, 83, 728-45.
Clarke, N. G. & Dawson, R. M. 1981. Alkaline O leads to N-transacylation. A new method for the quantitative deacylation of phospholipids. Biochem J, 195, 301-6.
Dang Y., Yang, Q., Xue, Z. & Liu, Y. 2011. RNA Interference in Fungi: Pathways, Functions, and Applications. Eukaryotic Cell, 10(9), 1148-1155.
Del Poeta, M., Nimrichter, L., Rodrigues, M. L. & Luberto, C. 2015. Correction: synthesis and biological properties of fungal glucosylceramide. PLoS Pathog, 11, e1004886.
Feldmesser, M., Kress, Y., Novikoff, P. & Casadevall, A. 2000. Cryptococcus neoformans is a facultative intracellular pathogen in murine pulmonary infection. Infect Immun, 68, 4225-37.
Huson, D. H., Richter, D. C., Rausch, C., Dezulian, T., Franz, M. & Rupp, R. 2007. Dendroscope: An interactive viewer for large phylogenetic trees. BMC Bioinformatics, 8, 460.
Huson, D. H. & Scornavacca, C. 2012. Dendroscope 3: an interactive tool for rooted phylogenetic trees and networks. Syst Biol, 61, 1061-7.
Kageyama-Yahara, N. & Riezman, H. 2006. Transmembrane topology of ceramide synthase in yeast. Biochem J, 398, 585-93.
Kim, H. J., Qiao, Q., Toop, H. D., Morris, J. C. & Don, A. S. 2012. A fluorescent assay for ceramide synthase activity. J Lipid Res, 53, 1701-7.
Lewis R. E., Kontoyiannis D. P. 2001. Rationale for combination antifungal therapy. Pharmacotherapy 21:149S.
Li, S., Du, L., Yuen, G. & Harris, S. D. 2006. Distinct ceramide synthases regulate polarized growth in the filamentous fungus Aspergillus nidulans. Mol Biol Cell, 17, 1218-27.
Luberto, C., Toffaletti, D. L., Wills, E. A., Tucker, S. C., Casadevall, A., Perfect, J. R., Hannun, Y. A. & Del Poeta, M. 2001. Roles for inositol-phosphoryl ceramide synthase 1 (IPC1) in pathogenesis of C. neoformans. Genes Dev, 15, 201-12.
Munshi, M. A. et al. 2018. The Role of Ceramide Synthases in the Pathogenicity of Cryptococcus neoformans. Cell Reports, 22, 6, 1392-1400.
Perlin, D. S., Brown, C. L. & Haber, J. E. 1988. Membrane potential defect in hygromycin Bresistant pma1 mutants of Saccharomyces cerevisiae. J Biol Chem, 263, 18118-22.
Rajasingham, R., Smith, R. M., Park, B. J., Jarvis, J. N., Govender, N. P., Chiller, T. M., Denning, D. W., Loyse, A. & Boulware, D. R. 2017. Global burden of disease of HIV associated cryptococcal meningitis: an updated analysis. Lancet Infect Dis, 17, 873-881.
Rittenour, W. R., Chen, M., Cahoon, E. B. & Harris, S. D. 2011. Control of glucosylceramide production and morphogenesis by the Bar1 ceramide synthase in Fusarium graminearum. PLoS One, 6, e19385.
Rittershaus, P. C., Kechichian, T. B., Allegood, J. C., Merrill, A. H., Jr., Hennig, M., Luberto, C. & Del Poeta, M. 2006. Glucosylceramide synthase is an essential regulator of pathogenicity of Cryptococcus neoformans. J Clin Invest, 116, 1651-9.
Schmidt, H. A., Strimmer, K., Vingron, M. & Von Haeseler, A. 2002. TREE-PUZZLE: maximum likelihood phylogenetic analysis using quartets and parallel computing. Bioinformatics, 18, 502-4.
Schmidt, H. A. & Von Haeseler, A. 2007. Maximum-likelihood analysis using TREE-PUZZLE. Curr Protoc Bioinformatics, Chapter 6, Unit 6 6.
Singh, A. & Del Poeta, M. 2011. Lipid signalling in pathogenic fungi. Cell Microbiol, 13, 177-85.
Soteropoulos, P., Vaz, T., Santangelo, R., Paderu, P., Huang, D. Y., Tamas, M. J. & Perlin, D. S. 2000. Molecular characterization of the plasma membrane H(+)-ATPase, an antifungal target in Cryptococcus neoformans. Antimicrob Agents Chemother, 44, 2349-55.
Ternes, P., Wobbe, T., Schwarz, M., Albrecht, S., Feussner, K., Riezman, I., Cregg, J. M., Heinz, E., Riezman, H., Feussner, I. & Warnecke, D. 2011. Two pathways of sphingolipid biosynthesis are separated in the yeast Pichia pastoris. J Biol Chem, 286, 11401-14.
Tidhar, R., Sims, K., Rosenfeld-Gur, E., Shaw, W. & Futerman, A. H. 2015. A rapid ceramide synthase activity using NBD-sphinganine and solid phase extraction. J Lipid Res, 56, 193-9.

What is claimed is:

1. A method of inhibiting the growth of a fungus comprising contacting the fungus with an effective amount of an inhibitor so as to thereby inhibit the growth of the fungus,
wherein the inhibitor inhibits ceramide synthase 1 (Cer1) in the fungal cells of the fungus.

2. A method of treating a subject afflicted with a fungal infection comprising administering to the subject an effective amount of an inhibitor so as to treat the subject afflicted with the fungal infection,
wherein the inhibitor inhibits ceramide synthase 1 (Cer1) in the fungal cells of the fungus.

3. The method of claim 1 or 2, wherein the inhibitor inhibits Cer1 activity or inhibits Cer1 expression.

4. The method of any one of claims 1-3, wherein the inhibitor inhibits Cer1 without substantially inhibiting a human ceramide synthase.

5. A method of inhibiting fungal ceramide synthase 1 (Cer1) activity comprising contacting the Cer1 with an effective amount of an inhibitor.

6. The method of claim 5, wherein the Cer1 is in a fungal cell.

7. The method of any one of claim 1-4 or 6, wherein the inhibitor inhibits fungal synthesis of ceramides and/or glucosylceramides.

8. The method of any one of claim 1-4 or 6-7, wherein the fungus is Cryptococcus neoformans, Blastomyces dermatitidis, Cryptococcus gattii, Candida albicans, Candida auris, Candida krusei, Candida glabrata, Candida parapsilosis, Candida guilliermondii, Coccidioides immitis, Aspergillus fumigatus, Pichia kudriavzevii, Rhizopus oryzae, Rhizopus spp., Histoplasma capsulatum, Coccidioides spp., Paecilomyces variotii, Pneumocystis murina, Pneumocystis jiroveci, Scedosporium spp., Sporotrix spp. Aspergillus spp., a dimorphic fungi or a mucorales fungi.

9. The method of any one of claims 1-8, wherein the inhibitor is a small molecule, a synthetic small molecule, a peptide, a protein, an anti-sense oligonucleotide or an RNA molecule.

10. The method of any one of claims 1-8, wherein the inhibitor comprises a CRISPR nuclease.

11. The method of any one of claims 1-8, wherein the inhibitor comprises a CRISPR nuclease; and a gRNA or sgRNA.

12. The method of any one of claims 1-8, wherein the inhibitor comprises a CRISPR nuclease; an RNA guide molecule; and a tracrRNA.

13. A method for inhibiting expression of a fungal ceramide synthase 1 (Cer1) in a fungal cell, the method comprising delivering to the fungal cell an RNA molecule, thereby inhibiting expression of the Cer1.

14. The method of claim 13, wherein the RNA molecule is siRNA, shRNA, dsRNA, gRNA or sgRNA molecule.

15. The method of claim 13 or 14, wherein the RNA molecule comprises a sequence that is complementary to a sequence in the target fungal Cer1 gene.

16. The method of claim 8, wherein the inhibitor is a small molecule.

17. The method of claim 8, wherein the inhibitor is a synthetic small molecule.

18. The method of claim 17, wherein the synthetic small molecule has the structure: 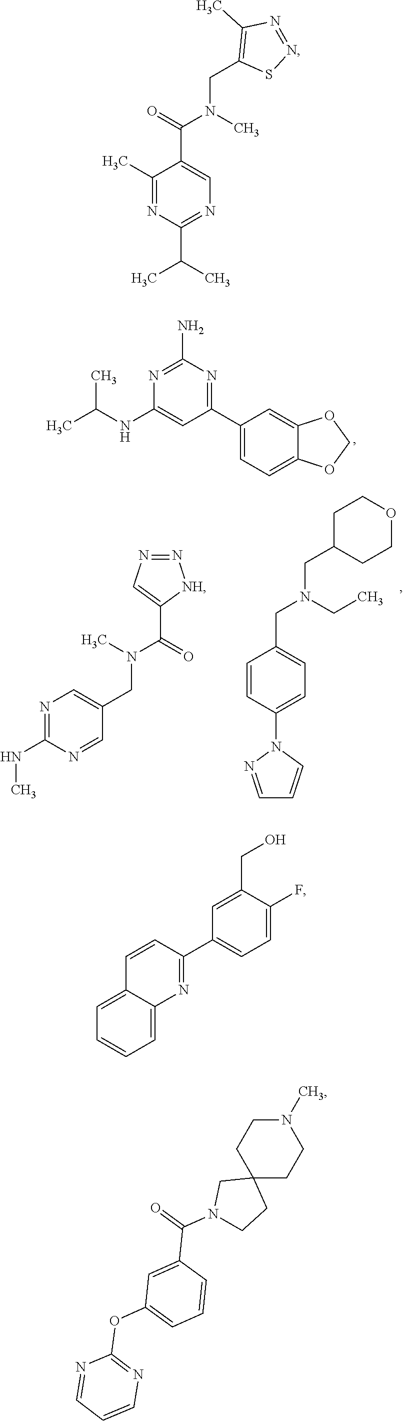 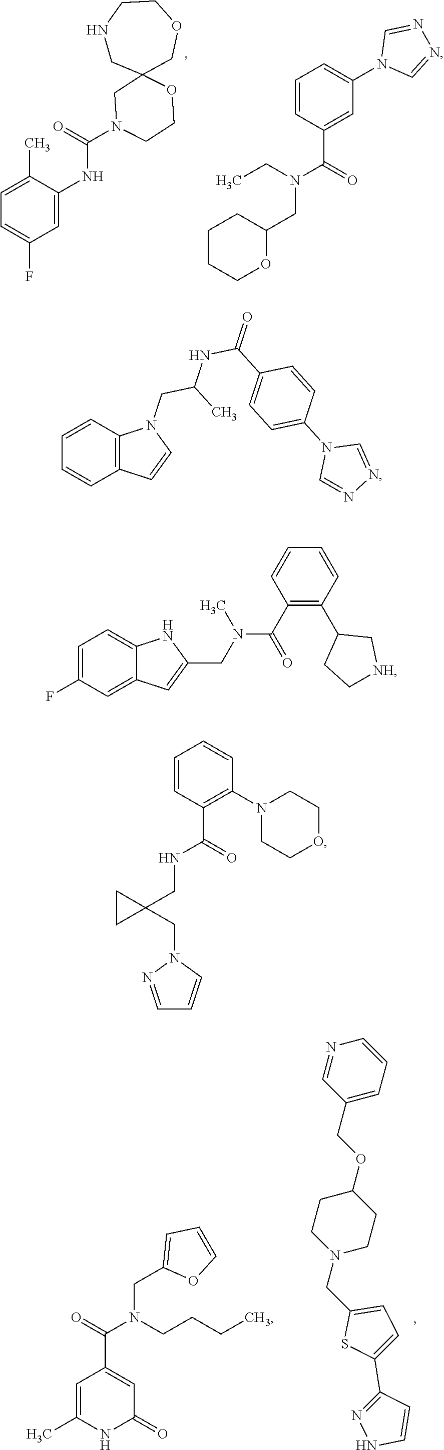 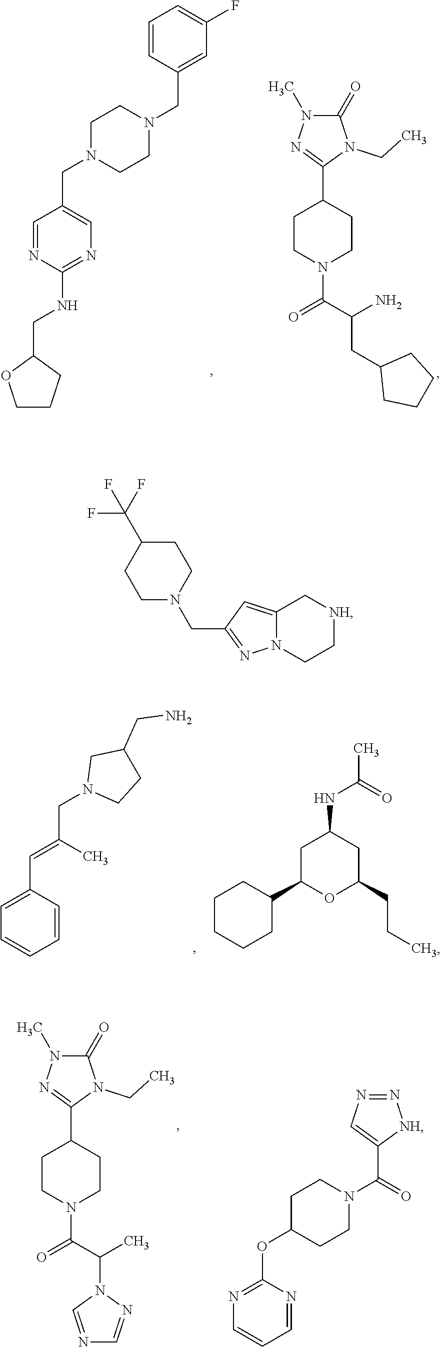 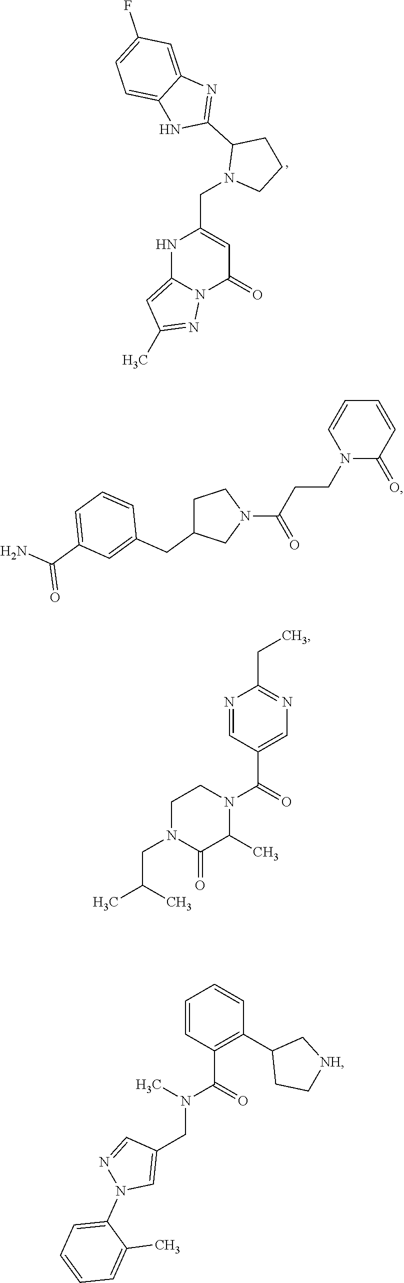 [SMILES: Cc1cc2[nH]c(CN3CCCC3c3nc4cc(F)ccc4[nH]3)cc(=O)n2n1 ; NC(=O)c1cccc(CC2CCN(C(=O)CCn3ccccc3=O)C2)c1 ; CCc1ncc(C(=O)N2CCN(CC(C)C)C(=O)C2C)cn1 ; Cc1ccccc1-n1cc(CN(C)C(=O)c2ccccc2C2CCNC2)cn1] 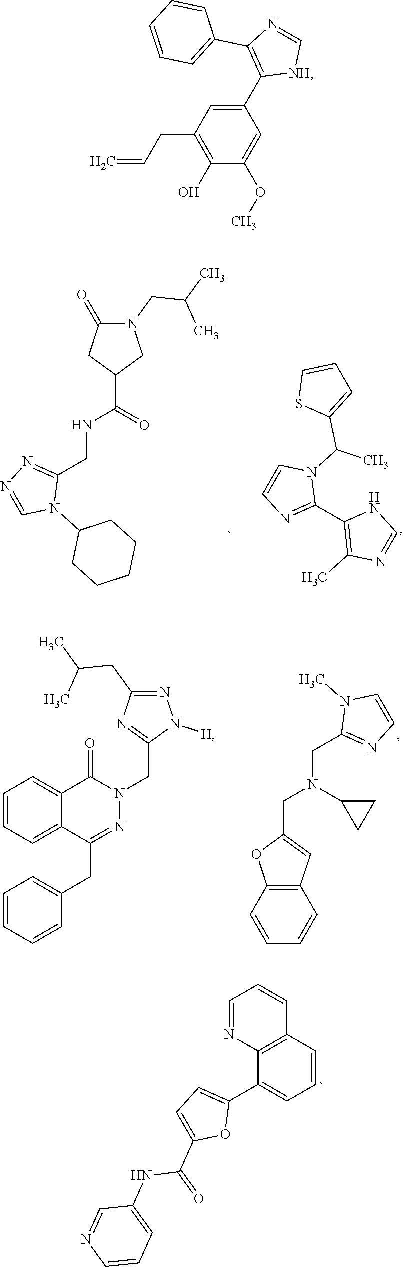 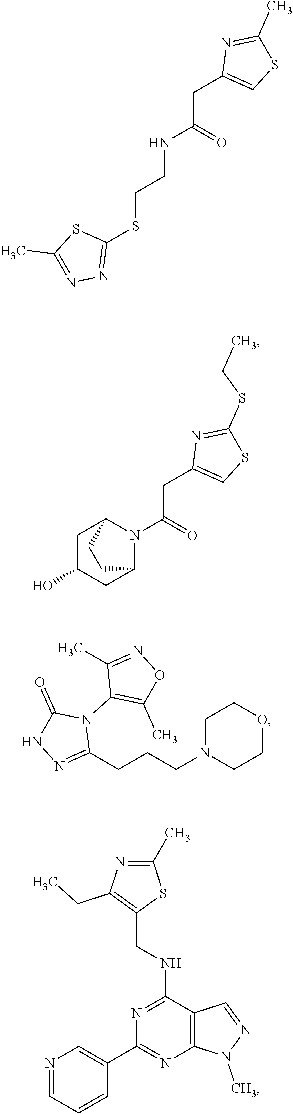 [SMILES: Cc1nc(CC(=O)NCCSc2nnc(C)s2)cs1 ; CCSc1nc(CC(=O)N2[C@@H]3CC[C@H]2C[C@@H](O)C3)cs1 ; Cc1noc(C)c1-n1c(CCCN2CCOCC2)n[nH]c1=O ; CCc1nc(C)sc1CNc1nc(-c2cccnc2)nc2c1cnn2C] 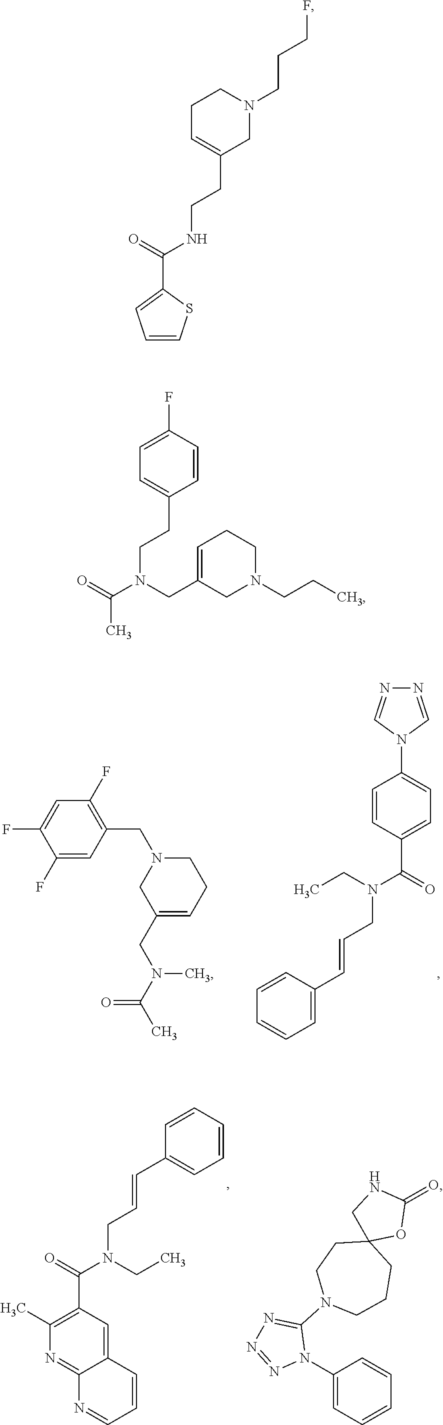 [SMILES: O=C(NCCC1=CCCN(CCCF)C1)c1cccs1 ; CCCN1CCC=C(CN(CCc2ccc(F)cc2)C(C)=O)C1 ; CC(=O)N(C)CC1=CCCN(Cc2cc(F)c(F)cc2F)C1 ; CCN(C/C=C/c1ccccc1)C(=O)c1ccc(-n2cnnc2)cc1 ; CCN(C/C=C/c1ccccc1)C(=O)c1cc2cccnc2nc1C ; O=C1NCC2(CCCN(c3nnnn3-c3ccccc3)CC2)O1] 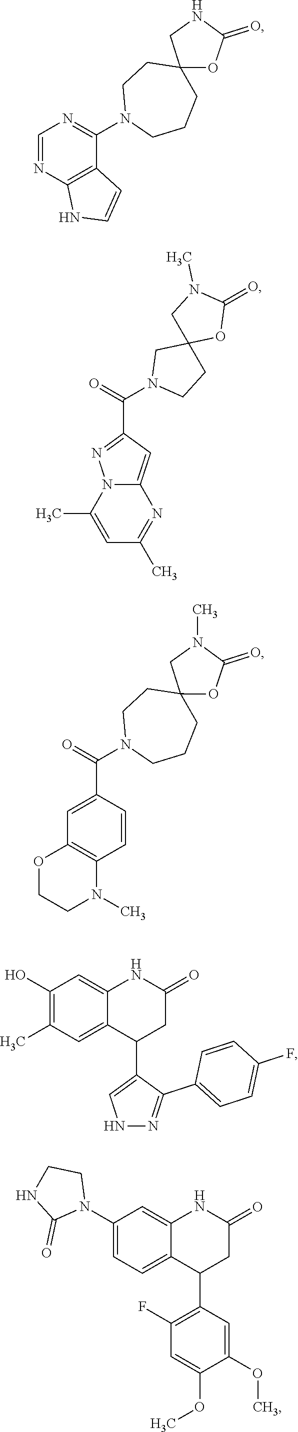 [SMILES: O=C1NCC2(CCCN(c3ncnc4[nH]ccc34)CC2)O1 ; Cc1cc(C)n2nc(C(=O)N3CCC4(CN(C)C(=O)O4)C3)cc2n1 ; CN1CC2(CCCN(C(=O)c3ccc4c(c3)OCCN4C)CC2)OC1=O ; Cc1cc2c(cc1O)NC(=O)CC2c1c[nH]nc1-c1ccc(F)cc1 ; COc1cc(F)c(C2CC(=O)Nc3cc(N4CCNC4=O)ccc32)cc1OC] 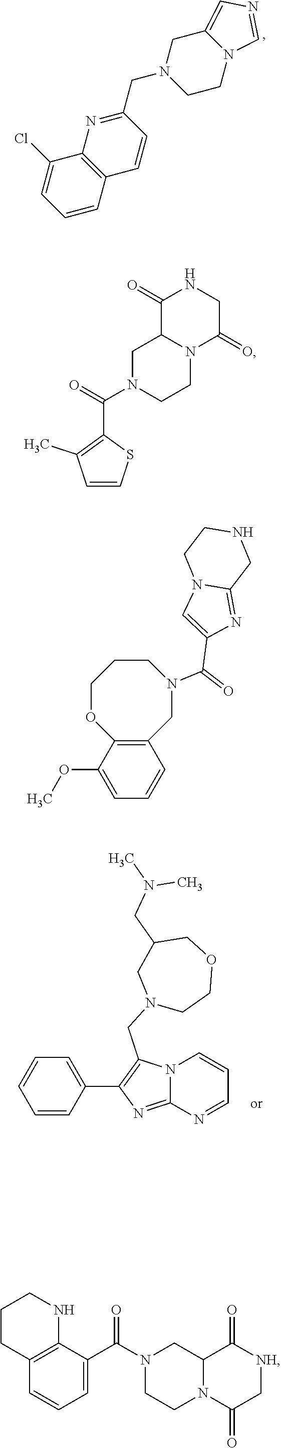 [SMILES: Clc1cccc2ccc(CN3CCn4cncc4C3)nc12 ; Cc1ccsc1C(=O)N1CCN2C(=O)CNC(=O)C2C1 ; COc1cccc2c1OCCCN(C(=O)c1cn3c(n1)CNCC3)C2 ; CN(C)CC1COCCN(Cc2c(-c3ccccc3)nc3ncccn23)C1 ; O=C1NCC(=O)N2CCN(C(=O)c3cccc4c3NCCC4)CC12]
or a pharmaceutically acceptable salt thereof.

19. A method for inhibiting expression of a fungal ceramide synthase 1 (Cer1) in a fungal cell, the method comprising delivering to the fungal cell:
a CRISPR nuclease;
an RNA guide molecule;
and a tracrRNA,
wherein RNA molecule comprises a sequence that is complementary to a sequence in the target fungal Cer1 gene.

20. A method for inhibiting expression of a fungal ceramide synthase 1 (Cer1) in a fungal cell, the method comprising delivering to the fungal cell a CRISR nuclease that targets a sequence of the Cer1 gene, thereby inhibiting expression of the fungal ceramide synthase 1 (Cer1).

21. A method of identifying an agent that inhibits the growth of a fungus comprising:
(ii) determining whether the agent inhibits fungal ceramide synthase 1 (Cer1),
wherein the presence of fungal ceramide synthase 1 (Cer1) inhibitory activity identifies the agent which inhibits the growth of the fungus.

22. The method of claim 21, further comprising:
(ii) determining whether the agent inhibits a human ceramide synthase,
wherein the presence of fungal ceramide synthase 1 (Cer1) inhibitory activity and the absence of substantial human ceramide synthase inhibitory activity identifies the agent which inhibits the growth of the fungus in the human subject.

23. A method of identifying an antagonist of fungal ceramide synthase 1 (Cer1) comprising:
(iii) contacting a fungal cell which expresses the Cer1 with an agent, and
(iv) determining whether said agent inhibits the Cer1,
wherein an agent that inhibits the Cer1 is an antagonist of the Cer1.

24. An inhibitor of fungal ceramide synthase 1 (Cer1) activity.

25. The inhibitor of claim 24, wherein the inhibitor is a small molecule or a synthetic small molecule.

26. The inhibitor of claim 24, wherein the inhibitor is a peptide or protein.

27. The inhibitor of any one of claims 24-26, wherein the inhibitor acts directly on fungal ceramide synthase 1.

28. The inhibitor of any one of claim 24-26, wherein the inhibitor acts downstream of fungal ceramide synthase 1.

29. The inhibitor of any one of claim 24-26, wherein the inhibitor acts upstream of fungal ceramide synthase 1.

30. The inhibitor of any one of claims 24-29, wherein the inhibitor targets a polypeptide or protein comprising or consisting of SEQ ID NO: 9.

31. The inhibitor of claim 24, wherein the inhibitor is an anti-sense oligonucleotide.

32. The inhibitor of claim 24, wherein the inhibitor is an RNA molecule.

33. The inhibitor of claim 24, wherein the inhibitor is an siRNA, shRNA, dsRNA, gRNA or sgRNA molecule

34. The inhibitor of claim 24 or 32-33, wherein the inhibitor comprises a CRISPR nuclease.

35. The inhibitor of claim 34, wherein the inhibitor comprises a CRISPR nuclease and a gRNA or sgRNA.

36. The inhibitor of claim 35, wherein the inhibitor comprises a CRISPR nuclease; an RNA guide molecule; and a tracrRNA.

37. The inhibitor of any one of claims 30-36, further comprising a gene knockout cassette.

38. The inhibitor of any one of claims 30-37, wherein the nucleotide sequence of the RNA, siRNA, shRNA, dsRNA, gRNA, or sgRNA molecule comprises or consists of a nucleotide sequence as set forth in SEQ ID NO: 1, a nucleotide sequence complementary to the nucleotide sequence as set forth in SEQ ID NO: 1, or a nucleotide sequence lacking one or more nucleotides from the 5′ end of SEQ ID NO: 1.

39. The inhibitor of 24, wherein the synthetic small molecule has the structure: 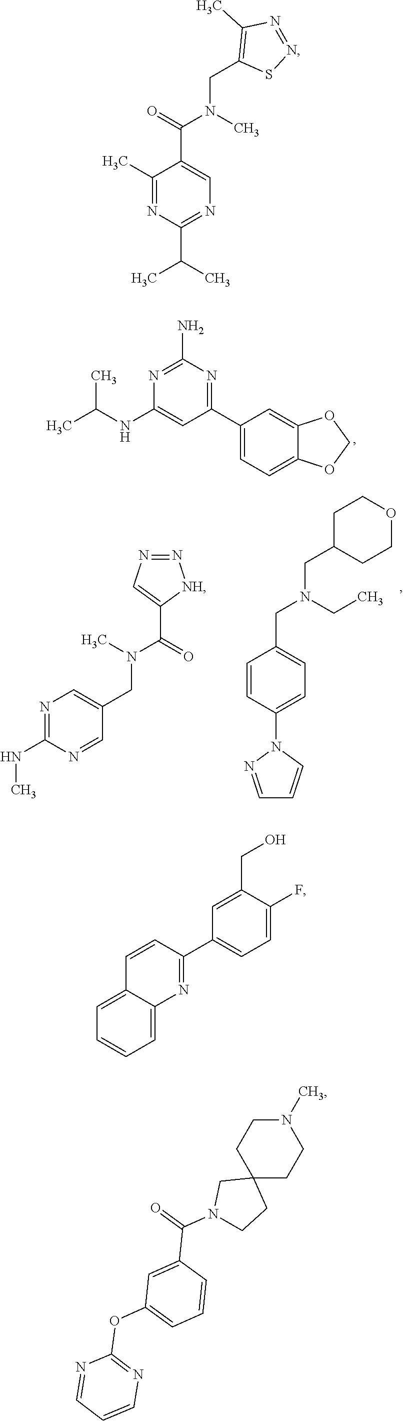 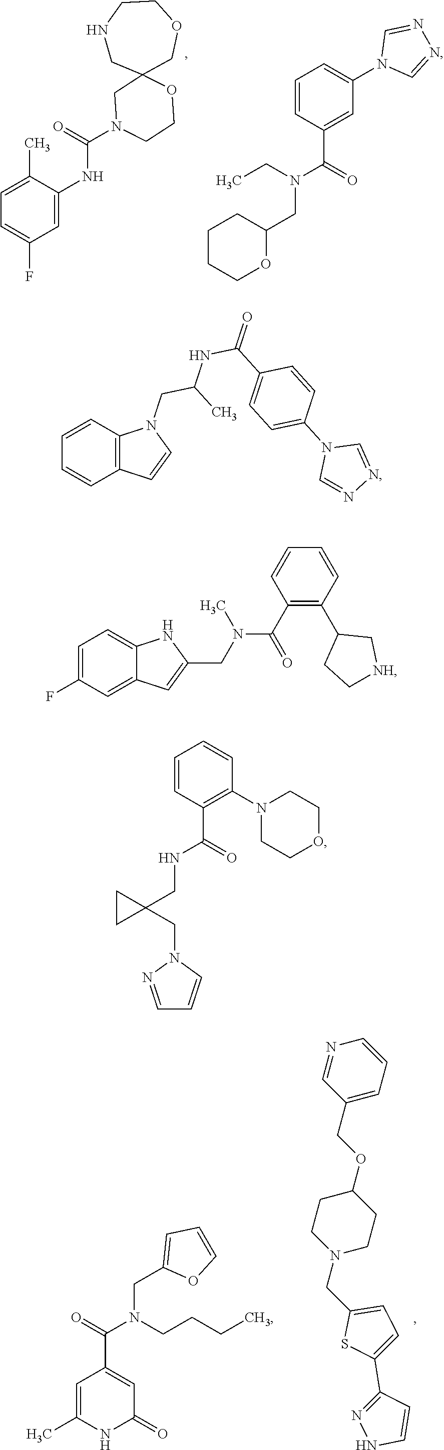 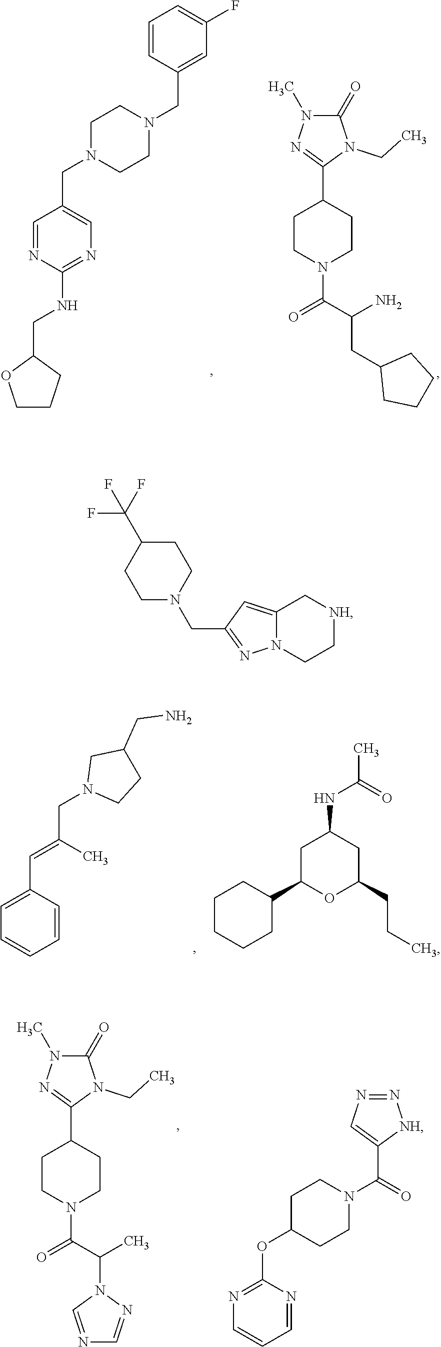 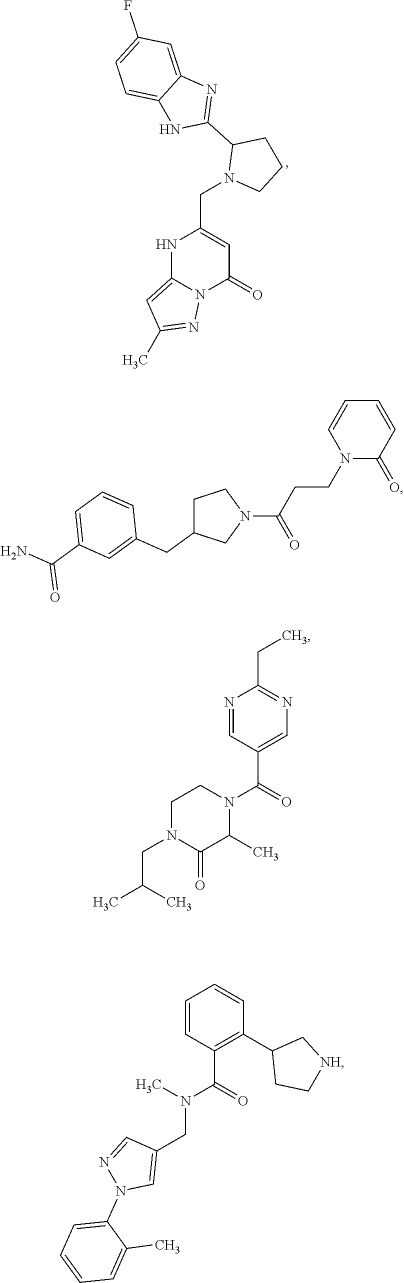 [SMILES: Cc1cc2[nH]c(CN3CCCC3c3nc4cc(F)ccc4[nH]3)cc(=O)n2n1 ; NC(=O)c1cccc(CC2CCN(C(=O)CCn3ccccc3=O)C2)c1 ; CCc1ncc(C(=O)N2CCN(CC(C)C)C(=O)C2C)cn1 ; Cc1ccccc1-n1cc(CN(C)C(=O)c2ccccc2C2CCNC2)cn1] 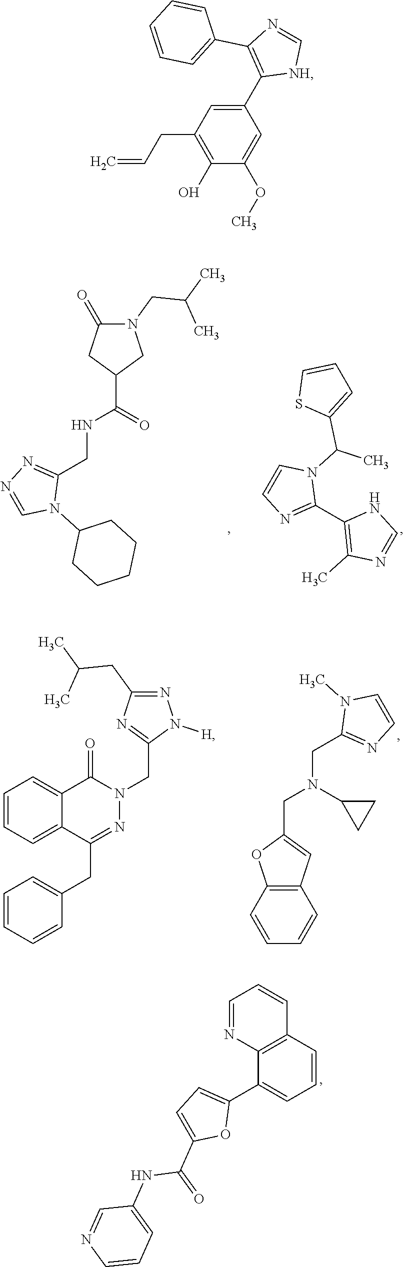 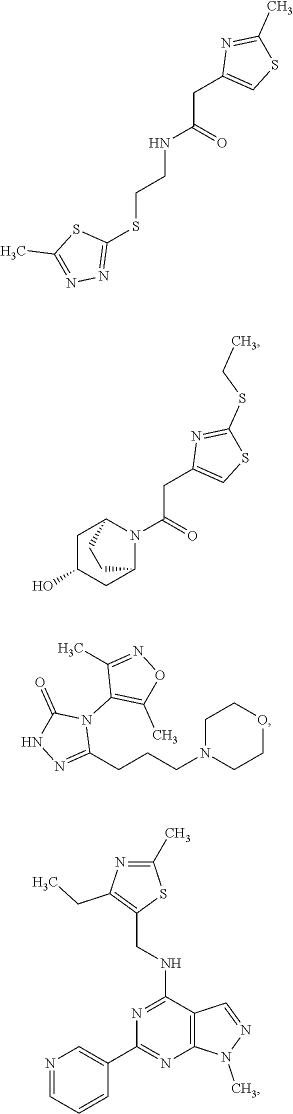 [SMILES: Cc1nc(CC(=O)NCCSc2nnc(C)s2)cs1 ; CCSc1nc(CC(=O)N2[C@@H]3CC[C@H]2C[C@@H](O)C3)cs1 ; Cc1noc(C)c1-n1c(CCCN2CCOCC2)n[nH]c1=O ; CCc1nc(C)sc1CNc1nc(-c2cccnc2)nc2c1cnn2C] 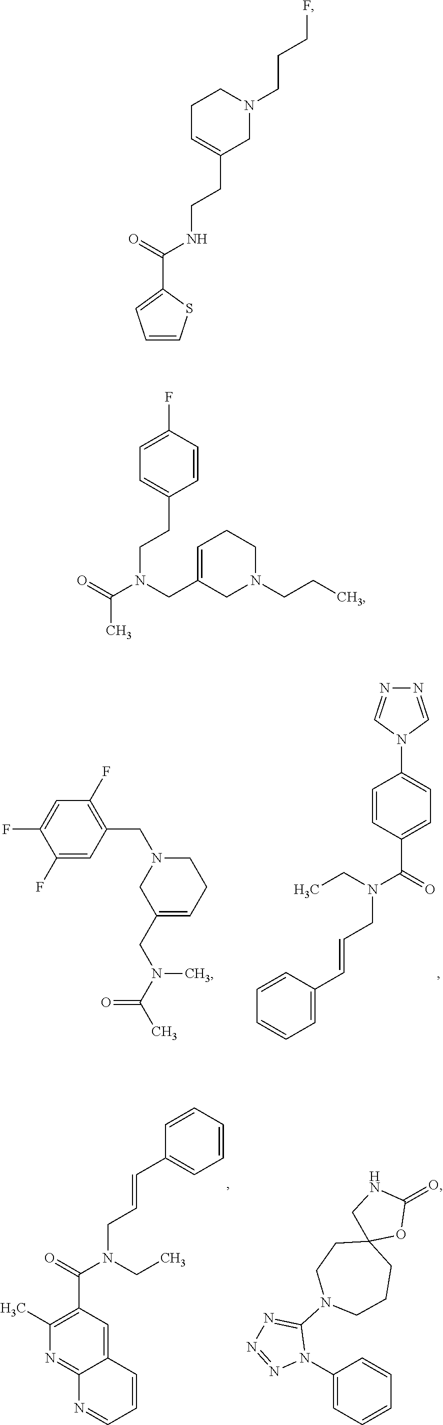 [SMILES: O=C(NCCC1=CCCN(CCCF)C1)c1cccs1 ; CCCN1CCC=C(CN(CCc2ccc(F)cc2)C(C)=O)C1 ; CC(=O)N(C)CC1=CCCN(Cc2cc(F)c(F)cc2F)C1 ; CCN(C/C=C/c1ccccc1)C(=O)c1ccc(-n2cnnc2)cc1 ; CCN(C/C=C/c1ccccc1)C(=O)c1cc2cccnc2nc1C ; O=C1NCC2(CCCN(c3nnnn3-c3ccccc3)CC2)O1] 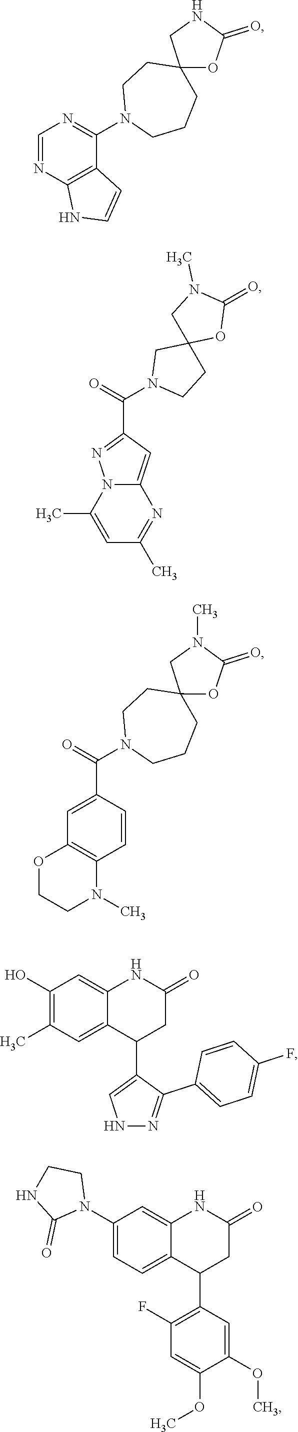 [SMILES: O=C1NCC2(CCCN(c3ncnc4[nH]ccc34)CC2)O1 ; Cc1cc(C)n2nc(C(=O)N3CCC4(CN(C)C(=O)O4)C3)cc2n1 ; CN1CC2(CCCN(C(=O)c3ccc4c(c3)OCCN4C)CC2)OC1=O ; Cc1cc2c(cc1O)NC(=O)CC2c1c[nH]nc1-c1ccc(F)cc1 ; COc1cc(F)c(C2CC(=O)Nc3cc(N4CCNC4=O)ccc32)cc1OC] 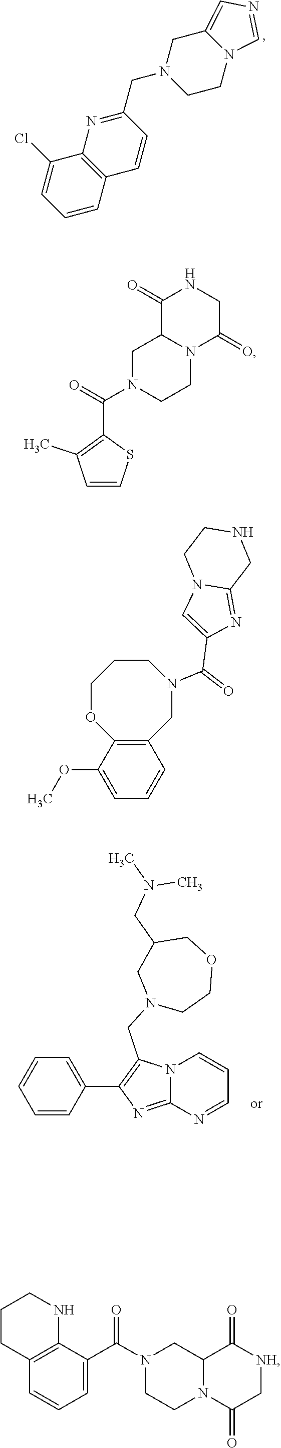 [SMILES: Clc1cccc2ccc(CN3CCn4cncc4C3)nc12 ; Cc1ccsc1C(=O)N1CCN2C(=O)CNC(=O)C2C1 ; COc1cccc2c1OCCCN(C(=O)c1cn3c(n1)CNCC3)C2 ; CN(C)CC1COCCN(Cc2c(-c3ccccc3)nc3ncccn23)C1 ; O=C1NCC(=O)N2CCN(C(=O)c3cccc4c3NCCC4)CC12]
or a salt thereof.

40. The inhibitor of any one of claims 24-39, wherein the inhibitor inhibits Cer1 activity or Cer1 expression.

41. A method of identifying an agent that inhibits the activity of fungal ceramide synthase 1 (Cer1) comprising:
(i) contacting the Cer1 with the agent and separately with the compound of claim 39 or salt thereof; and
(ii) comparing the Cer1 inhibitory activity of the agent with the Cer1 inhibitory activity of the compound to identify the agent with Cer1 inhibitory activity that is greater than that of the compound.


SEQUENCE LISTING (16 sequences)

SEQ ID NO: 1 - DNA, 1470 bp, Cryptococcus neoformans
gttacgtgaactgtccagtccacagccaagtgcagccttttccttccccgtcaaaaaccacccccccctccccccctgcacaggtaaccaaacacccacgagctggcttcgtggactgcactgttcccttccccccacgcgacatccgcacctgctgcagcagcgcactccgccagcatggcgtgcaacctcaccacctcccccctcccctccctcctccactcctatgtcccacagtcgctgcggccctttgtcaccctgtcgtatcccatcacagacccgctcgcccacccgtccgcgcagacactgtacgacaagggcccgcaggatgcgtgcttggtgctcttctgggcgctggcgttcaccctgctccgggaggcgttcatgaagggtgtcttctctccctttatgagaatatgtctgccaagcccgccacagatcaagggacaggagagagaatatgcaaaggccaggaagaagagggagcataccgtcacgaggtttgcagagcaaggctggagctggctgtattgctctatatattggacatttggcgtgattgtcctccgccagaatccctctccaacatctcccgaacagctctggggcacatacccggccgtccctctcccggcactcaccaagttttactatctctcccagctcggttggtggttccaccagctgctcgttatcaactgcgagaagcgaagaaaagatcattggcagatgtttgggcaccacatattgactattactttggttgtggggagctacgtcatgaactttacctcggtcggtgtcgttatccactgtcttatggacttttgcgacatcctcttaccactcgccaaaatgtttcgctacctctccctctccaccctctgcgacctcacattcgtcgtctttctcatctcatggttcatcactcgtgaagccggtctcttcctcgtcatccgcagcacctatgtggacgcgcccaaattcatcccctttgaatgggctcccgaacaaggccgattccttacttacagggtttacctcggctttgtcgccatgctctccatcctctggatcctcgccacggcgtggttttacatggcttgcaacgtcgcgatccgcgtggtacgaggtatgggcgctgaagactcgagaagtgatgatgagtccgaagaggatgcgttggagcaagtgccagagagtgtaggtgcttcaactgcgaccaggtctgaggtagaagaggatctgcgaaaacgaaaattattttctcaacgagggacttgatgatagattatttttgatatgaagggacgtttttcaaaaaaaaaaaaaaaggaaaaaaaaaatagacacaaaaaaagatcagtgtatacctctaaaatagattgaaaaagtatcctcaattttttaacgcaggattttttaacgccgggatgatcatcacatgcattagacgtcatatataaaaca

SEQ ID NO: 2 - DNA, 1404 bp, Aspergillus fumigatus
atggcaaaagacgaatccacgcgatcgcaatccgtttcggagcttaatacttgtgtgtccaccagtcaccgaactgctgccaaagacgcgacctttcgagaatggatggacgcgaatcaaattggtatgcgtcttttctccaacttgccttggagttggactcgatggctgaccgttgctctgtatgcgtatctaggagtctcattgactattcttggcacccttttggctgctcataacctctaccccaccctccggccatacacggcgccgttcttcgaattgtcgtactaccaaccttccgaaggggtctatgtccaaggatttgacgatgtgtactttgtcgtcagctccgccatcacgttcaccgccgtccgagcgattgccattgaatggatcttcagaccagccgccagatatgcgggattgaagcggaaggcttcaaatcgttttgccgagcaggcgtggatgtggatgtactatgcgttcttctggacgtttggaatgtatatctggacaaactcctattactggatggatttcaaggcaatttgggctcaatggcccgcgcggggtatctctgcaaacctgaagtggtatctgctggcacagctgtcattctggttccagcagatcctggttatcaacatggaagaaaggagaaaagatcactaccagatgttgactcaccacatcatcaccagcactctcttgacgtccgcctatatttacggtttttataacgtttcaaacgtggtcctgtgtctgatggacattgtcgacctgttgctgcctactgccaagattctcaagtacttcaagttcgagctttgctgcaacatcaccttcggcctgttcatggtgacctggttgattacacgtcacattttctacccactactctgctggtctatctacaaggatgtccctgatgccatggcctacggctgctactccggtagcaccgcggaaatgatttcaaccgatgggtacccgaaccgattccgctacctttttaaccccttcttggacatcaatggccccatctgcatgaatcgcactgtcaagtggattttccttggttttcttctggcgttgcaattgctctctctcatatggttcgtgatggttgtccgcgtggctgtaaatgtgctccgaaccggaaatgccgaagacactcgaagcgacggcgaagaggaagaggaagaagtcgaagtccgacctgtgggcaaggacgcgctcaacaacaacccagtcggtgtggacggggcgaatgccgactggcgtcgagcctcgagcggctcagctagcgtgcgacctcgcgcgcggggacgaatcccacttggtgaccagagcgatcgcaaggccctccttggcaggatcggatgcgacaagccgacgtag

SEQ ID NO: 3 - DNA, 1135 bp, Candida auris
atgaagctcacagcctcgcaaaggcaaaagctgaatcagcgtgtgcgtgctttgagcgatgaatcaaggaccgatgttgcaataatacgcaaaatgtttgttgcatttaaggaattgtcattcagacacacttggataaatccgttcattatcctttcggtgttctacatttcgtactttggtactactgaatcaaacccgattcatgaaaatctcaaaaaactcattaaaccatcgtaccatatcgttggaacagatcagtatggaaaaggtgtgaatgacttctattttgttgcattttacgctcttttttttacatttttgagagaatttatgatgtgcgtaattttgaggcctctcgcgggcatgttaggtgtcacaaggccgcacaaagttaatagatttatggagcaaagttacgcaatgttctattatggtttgtctggcccctatggactttatgttatgtccagaatgccactttggttttttgagactactcctttgtacgaaagttaccctcacaagacgcatgattggcttttcaaagtatactacctcggacaggcggccttttgggttcaacaatccgttgttctaattctccagcttgaaaaacctagaaaggacttccatgagcttattcttcaccacattatcacgattgcgctcatttggttgtcctacagatttcatttcacgtggatggggctcgaaatttatgttaccatggatgtctcggacttttttttagcaacttcgaaaacgttgaactatctcgattcgattctcacagggccatttttaataggctttgtatttatttggatctatctcagacactatgtgaatctaaaaatattatggtctgtactcactgagtttaaaaccgttggtgagtgggagttgaattgggaaacgcagcaatacaaatgttggatttctcagcccattgtctttgggctcatttttgcattgcaagtcttgaatgcttattggttattcctaatattgagaattttgtatagatacgttgttggcggcgttaaagcagacgaaagaagtgagtcagaagatgaggatgaagatgaacctcctcagaaggataaatctgtatcagaaggataaatctgta

SEQ ID NO: 4 - DNA, 1434 bp, Balstomyces dermatitidis
gccgcaaaaatccttaaatacctcggatacgaacgcgcctgcaccgtagggttcatagtcttcctcgtcacctgggtaatatcgcgccacatcatctataacctcctttggtggtctatctacgtcaacgtccccgacgttatgccctacggctgctactccgccacgacaacgaagatgatctcacccgctgccaacaccaccctggacaacggcgccgcatccatagacctaaacaactggaaccacctcctccacccattccaagacctgggcggccgcatctgcatgagcccgcgcatcaaatgggtgttcctctctttcttgcttttcctacagatcctcgcccttatatggttcacgatgattctgcgtgtggctgttaaggtgctgaagagcgggtcagcggaggactcacggagcgatgacgaaggggaggaggaagaagaagtgaattcggagactacgaggatgggtggaaatgggacaattgctggtgttcgtgatgggattaatggtaatgggaccgcgatcgtttcgggcagttctacttcatcgtcaaccgcgcagggtcatcatcctgtgcgaattcggacgggtcggggccgggttacgttgagtgatcagaatgaccggaaagcgttgttggggcgcattgggtgtgataaaccctcatgcctttggtaccattctaaatactggaataatttccgcgagatatggaccgactggccttccagagacatctccggcgtcttcaagtggtactgcctgacacagctggcgttctggttccaacagatcatcgtcatcaacattgaggagaggcggaaggattactatcagatgctggttcaccacatcgtcacaagcactcttttaggctctgcttatgtctatggcttctacaacgtcgccaatgtcgtcctctgtatcatggacatcgtcgatttcctgctgccggtaagacgcataggtatctgcatcaccatcctcacaatgatattcgccctgcacaatctctatccatctctaagaccgtacacctcccctttcctgcaattaccgcactaccagcctgagaagggtacatatgtccagggttgggatgatctttattttgccatgggaggcgttctggcattcactgccgtcagggcgatcgctgtcgagtggatcttccagccccttgcccgaagatacggtctgaagcataaggccgccgtccggttggcggaacaaggctggcttcttgtttactattttggtttctggacttacggagtggtaatggtgaagaccctctccagccagcccaattcagtttccgaagtcaatgcctgtgtttctcccatggaaggctgtcgggcggccaagatgaagccaaaagaggagaccaattttcgacaatgggtgctgcaaaatcagataggt

SEQ ID NO: 5 - DNA, 1395 bp, Histoplasma capsulatum
gccgcaaaaatcctcaaatacctcgggtatgaacgcgcctgcacggcgggcttcatagtcttccttgtcacttgggtaatctcccgccatatcgtctacaatctcctctggtggtctatctatatcaacgtcccagacgttatgccctacggttgctactctgcaactacaaccgagatgatttcacccgctgcaaacgccaccctagacggcgccgcatccatagacctaaacaactggagccacctcctccagccattccgagatctgggcggccgcatttgcatgagcccacgcgtcaaatgggttttcctctctttcctgttattcctgcagatcctcgcgattttatggttcacgatgattctacgtgtagctgtcaaggtgctgaagagtgggtcggcagaggattcgcggagtgatgatgaggaagaagatgaggaagaggtggattcgcaaaatttaaggacggaagggaatgctggtgttagggatggtggtcgtgggaatggcacggcgatcgtgtctgggagttctgtctcatcatccgcggggcaaggtcatcatcccgtgcgaatcaggaccggacgaggtcgagttaccttgagtgatcagaatgatcggaaagcgctgttgggacggattgggtgtgataaacccacgttcctttggtacaattctaagtactggtataatttccgcgagatatggaccgattggccctccagagatatctccggtatcttcaaatggtactgtctgacgcagctggctttctggttccaacagatccttgttatcaacattgaggagagacgcaaagattattgtcagatgctggtccatcacatagtcacaagcacgcttttagggtctgcttacgtctatggcttttataatgtcgccaacgtcgtcctctgcattatggacattgtcgattttctgctgccggtaagcaagataggtatctgcatcactaccctcacaatgatattcgcccttcacaacctctacccgtcgctgaggccgtacacctcccccttcctgcaattgccgcactaccagcctgagaagggtacatatgtccagggctgggatgatatctattttgttatggggggcgttctcgctttcaccgccgtccgagcgattgccatcgaatggatcttccagccccttgcccgcagatacggcttgaaacacaaagcatctgtccggctggcggagcaaggctggattctcgtttattattttggcttctgggcttacggagtgatgacgaaggtgaagccaacgccggagaccaattttcgacaatgggtgctgcagaaccagataggtacaatatacaaatgttcctcaatctttgtcccgcccaatatcccatcactaaaa

SEQ ID NO: 6 - DNA, 1666 bp, Coccidioides immitis
aactcttactcttcatatgctttgtttctgttcggtcggttctcagtacctttccggagtcacaagtagctcctttccgccgtctgaggaaattttctgagcaagcaacacaaaactcagagacaaattaatattctccttcctctcttccttctactcccccctctactcttgtaggcgcgatcttcctcgaattttcagtcctggatctcccgatacctcttcttgtcaacttcggcccacaagaacaaatcacgggctttcactcttgttgaggttgcgtggtgtcgggggcgccattgaaacagagtcgataatggcgaagccggcccgtagccattccaactctgttacagaggtcgaggcctgcgtttcgtcgccagaagggagagggtccatcaggaaagatgccagtcttcgagaacggctgctcagcaaccaaatcggaatctcccttaccattcttacgatgatcttcgcggttcacaacttatacccttcgcttagaccatacacgtctcctttcttaactttgccacattaccgatcgacaaaaggaatatacgtccagggatgggacgacttgtacttcatcatcggttccatggtcgcctttacagcaatccgagccatcgcaattgactggatccttatgcccatcgcacaacaacttgggctaaagttgaaggcatcgttgagattcgccgaacaaggctggctactggtgtattacattgtcttctggtcctatggtttgtatatttggatgaattcaaagtactggatggatttccgagaaatttggacggactggccgtcgagagagataccaggctatttcaagctgtattgcctgttgcagttgtcattttggttgcaacaaattttcgtcatcaacattgaggaaagaagaaaagaccattatcagatgttgacgcatcatatcgtcacaagcacccttctcggatcagcttatgtttacagcttctataacgtggccaacgttgttctatgtattatggatattgtagatttcctcctcccggctgcgaagatgctaaaatatatgggttacgagcgcatttgcaccattgcattcggggtcttcctagccacatggttcattgcccgccatgtgatttacatgatgttatggtggtccatttaccaaaatgttccagatgcaatgtcatttggatgttatctgggcgcaaccggccagaagcttattgacgtatctcccgattcctggggctcactcatatatccgtttcgggatatcgatggacctatttgcatgagtttccgcatcaaatgggcctttctcactctgctcctaatacttcagatgctctctcttatctggttcggcatgatccttcgtgtggcagtgcatgtgcttcggacaggctcatccgcggaggatacccgcagcgacgatgagggcgaagaatcaactgaggcagtccgccctgttcgccgaggcagccctagaagacaggaagattatgaagagaatggatggagtaaatctgttgcggtcaacggttccgcccagaatcatcacccggtgcgaatcagaacagctcgcggccgcgttacactcagcgatcagaacgaacgaaaggctttactcggcagaatcgggtgcgacaagccttcttaa

SEQ ID NO: 7 - DNA, 1080 bp, Pichia kudriavzevii
atgagacaaagatcggcattggaaaagcaacaaacaattgttgacaaggatgaaaaaaattcaaaatttatcccgaagggtaacaaacgtgaacaaaggcaagagcaaaagcgcctgtatgaaatacatcagatccaaaattcagaattagtcgataaagcaattataatttcatcactcattttttatttaatcttattcattgcatctcgatttgtgtctctcaaattcaagaaattttacaagttgtcatacaaatatgaagattctgattattatgacattggttttgacgatctttattttgggatattttggattatcaatttactatttttaaggagtttcctcattctattttgttttaatccatgcgcaaaactcttaggaatcaaaaagtttaaagccacccaaagatttattgaacaggcttggtcaatggtatactactcattttcttggggattcgggttctacctttactataattctgattattatttagactgctacaacatttatgccaattggccacatgacgtactctcagcacctatgaaattctactacttgcttcaatcagcgagctggttccaacaatttattgttctacatatagagtcaaggagaaaggaccattatcaaatgttagcccaccatatcatcacttgtattctaacaactgcttcttactcattatattttaccaagattggtcatgtgattcttttattaatggacattgtggatgtttttttgtcaactgccaaaatcttaaaatatgctggatttcaaacagtatgtgatttaatgtttctattttttatggtttcttggattatatttagacatggagtttataattacgttttatggtttacagcaacaagggctagagatataatgggaaataagtgctcaacatttttgcctagtgagacttataaagcatgttacacagatttacaggtggatatttttatgctactcctagttgcattacaagtgataatgtgtatctggatgtatatgatttttagagtcgctttccatgttatttcaggtggatccgcagatgat

SEQ ID NO: 8 - DNA, 1284 bp, Candida albicans
atgagttcaggatcaattgcaactacaacaatacccagtacaccaatgtctattgaatcaatagaagatcatcaatatcataattcttttttagccatggttgaaagaaatcaaataccgttatctagaaatctattaataattttatatttatcacatttattattacaaaataacaataccactattactccttatacatccaaatttatacacattcaaaattgtgttggtattgatgaattcactgggaaatcaatatatgatattgatattaatgatacatattttgtgattcattcattagtcattgttacatttcttcgttcattcttgatgaaatggtgttttgaaccatttgcatcgaaattttgtcatattcattcgaaaaaagccaaaactagatttgctgaacaaagttggtcatttgtttattattcaatttcatttatatttggcgtggtattatattgggatagtccttattataataatttagatcaagtttatatcaattggccaaatcattacatgtcatgggaatttaaaacttattatttagtaagtatgggattttggctacaacaaatttttgttttaaatgttgaaaaaccaagaaaagatcattatcaaatgtttagtcatcatatcattacttgtttattgattattggttcatattattattattatttccgtattggtcatttaatattgatgattatggattcagtagacatttttttggcagcagcaaaaatgttaaaatacgctgggtttagtaatgcatgtgatgccatgtttcttttatttttagtcagttggattgtattaagacatggagtatataattatatcttttatcatgcttggtataaatcagttgatttaatgaaaaatggacaatgtgttgaaggattgatgcaaaaaagatgttggactccagttgttattgatacatttttgggtttactcggaggattacaaattataacttgtatttggatgtatttgatattaaaagttgcttataaagttgttactggatcaggtgctgaagatgttcgaagtgatgaagatgacactgatattgagttggaagaagaagaaaaagaagaagaagaggaggaggaagtaggacaaccaatatttgtggaaaaaaaggaagaagaagttgtattagaaattgaagaagataaatactcttttgaaagagattcatttagttcatcatcagaatcaactttggacgagaaaaaggatattaggaaaagaaaagtataa

SEQ ID NO: 9 - protein, 368 aa, Cryptococcus neoformans
MACNLTTSPLPSLLHSYVPQSLRPFVTLSYPITDPLAHPSAQTLYDKGPQDACLVLFWALAFTLLREAFMKGVFSPFMRICLPSPPQIKGQEREYAKARKKREHTVTRFAEQGWSWLYCSIYWTFGVIVLRQNPSPTSPEQLWGTYPAVPLPALTKFYYLSQLGWWFHQLLVINCEKRRKDHWQMFGHHILTITLVVGSYVMNFTSVGVVIHCLMDFCDILLPLAKMFRYLSLSTLCDLTFVVFLISWFITREAGLFLVIRSTYVDAPKFIPFEWAPEQGRFLTYRVYLGFVAMLSILWILATAWFYMACNVAIRVVRGMGAEDSRSDDESEEDALEQVPESVGASTATRSEVEEDLRKRKLFSQRGT

SEQ ID NO: 10 - protein, 443 aa, Aspergillus fumigatus
MAKDESTRSQSVSELNTCVSTSHRTAAKDATFREWMDANQIGVSLTILGTLLAAHNLYPTLRPYTAPFFELSYYQPSEGVYVQGFDDVYFVVSSAITFTAVRAIAIEWIFRPAARYAGLKRKASNRFAEQAWMWMYYAFFWTFGMYIWTNSYYWMDFKAIWAQWPARGISANLKWYLLAQLSFWFQQILVINMEERRKDHYQMLTHHIITSTLLTSAYIYGFYNVSNVVLCLMDIVDLLLPTAKILKYFKFELCCNITFGLFMVTWLITRHIFYPLLCWSIYKDVPDAMAYGCYSGSTAEMISTDGYPNRFRYLFNPFLDINGPICMNRTVKWIFLGFLLALQLLSLIWFVMVVRVAVNVLRTGNAEDTRSDGEEEEEEVEVRPVGKDALNNNPVGVDGANADWRRASSGSASVRPRARGRIPLGDQSDRKALLGRIGCDKPT

SEQ ID NO: 11 - protein, 371 aa, Candida auris
MKLTASQRQKLNQRVRALSDESRTDVAIIRKMFVAFKELSFRHTWINPFIILSVFYISYFGTTESNPIHENLKKLIKPSYHIVGTDQYGKGVNDFYFVAFYALFFTFLREFMMCVILRPLAGMLGVTRPHKVNRFMEQSYAMFYYGLSGPYGLYVMSRMPLWFFETTPLYESYPHKTHWLFKVYYLGQAAFWVQQSVVLILQLEKPRKDFHELILHHIITIALIWLSYRFHFTWMGLEIYVTMDVSDFFLATSKTLNYLDSILTGPFLIGFVFIWIYLRHYVNLKILWSVLTEFKTVGEWELNWETQQYKCWISQPIVFGLIFALQVLNAYWLFLILRILYRYVVGGVKADERSESEDEDEDEPPQKDKSV

SEQ ID NO: 12 - protein, 472 aa, Balstomyces dermatitidis
MVKTLSSQPNSVSEVNACVSPMEGCRAAKMKPKEETNFRQWVLQNQIGICITILTMIFALHNLYPSLRPYTSPFLQLPHYQPEKGTYVQGWDDLYFAMGGVLAFTAVRAIAVEWIFQPLARRYGLKHKAAVRLAEQGWLLVYYFGFWTYGVCLWYHSKYWNNFREIWTDWPSRDISGVFKWYCLTQLAFWFQQIIVINIEERRKDYYQMLVHHIVTSTLLGSAYVYGFYNVANVVLCIMDIVDFLLPAAKILKYLGYERACTVGFIVFLVTWVISRHIIYNLLWWSIYVNVPDVMPYGCYSATTTKMISPAANTTLDNGAASIDLNNWNHLLHPFQDLGGRICMSPRIKWVFLSFLLFLQILALIWFTMILRVAVKVLKSGSAEDSRSDDEGEEEEEVNSETTRMGGNGTIAGVRDGINGNGTAIVSGSSTSSSTAQGHHPVRIRTGRGRVTLSDQNDRKALLGRIGCDKPS

SEQ ID NO: 13 - protein, 442 aa, Histoplasma capsulatum
MTKVKPTPETNFRQWVLQNQIGICITTLTMIFALHNLYPSLRPYTSPFLQLPHYQPEKGTYVQGWDDIYFVMGGVLAFTAVRAIAIEWIFQPLARRYGLKHKASVRLAEQGWILVYYFGFWAYGVFLWYNSKYWYNFREIWTDWPSRDISGIFKWYCLTQLAFWFQQILVINIEERRKDYCQMLVHHIVTSTLLGSAYVYGFYNVANVVLCIMDIVDFLLPAAKILKYLGYERACTAGFIVFLVTWVISRHIVYNLLWWSIYINVPDVMPYGCYSATTTEMISPAANATLDGAASIDLNNWSHLLQPFRDLGGRICMSPRVKWVFLSFLLFLQILAILWFTMILRVAVKVLKSGSAEDSRSDDEEEDEEEVDSQNLRTEGNAGVRDGGRGNGTAIVSGSSVSSSAGQGHHPVRIRTGRGRVTLSDQNDRKALLGRIGCDKPT

SEQ ID NO: 14 - protein, 449 aa, Coccidioides immitis
MAKPARSHSNSVTEVEACVSSPEGRGSIRKDASLRERLLSNQIGISLTILTMIFAVHNLYPSLRPYTSPFLTLPHYRSTKGIYVQGWDDLYFIIGSMVAFTAIRAIAIDWILMPIAQQLGLKLKASLRFAEQGWLLVYYIVFWSYGLYIWMNSKYWMDFREIWTDWPSREIPGYFKLYCLLQLSFWLQQIFVINIEERRKDHYQMLTHHIVTSTLLGSAYVYSFYNVANVVLCIMDIVDFLLPAAKMLKYMGYERICTIAFGVFLATWFIARHVIYMMLWWSIYQNVPDAMSFGCYLGATGQKLIDVSPDSWGSLIYPFRDIDGPICMSFRIKWAFLTLLLILQMLSLIWFGMILRVAVHVLRTGSSAEDTRSDDEGEESTEAVRPVRRGSPRRQEDYEENGWSKSVAVNGSAQNHHPVRIRTARGRVTLSDQNERKALLGRIGCDKPS

SEQ ID NO: 15 - protein, 367 aa, Pichia kudriavzevii
MRQRSALEKQQTIVDKDEKNSKFIPKGNKREQRQEQKRLYEIHQIQNSELVDKAIIISSLIFYLILFIASRFVSLKFKKFYKLSYKYEDSDYYDIGFDDLYFGIFWIINLLFLRSFLILFCFNPCAKLLGIKKFKATQRFIEQAWSMVYYSFSWGFGFYLYYNSDYYLDCYNIYANWPHDVLSAPMKFYYLLQSASWFQQFIVLHIESRRKDHYQMLAHHIITCILTTASYSLYFTKIGHVILLLMDIVDVFLSTAKILKYAGFQTVCDLMFLFFMVSWIIFRHGVYNYVLWFTATRARDIMGNKCSTFLPSETYKACYTDLQVDIFMLLLVALQVIMCIWMYMIFRVAFHVISGGSADDVRSDTDD

SEQ ID NO: 16 - protein, 427 aa, Candida albicans
MSSGSIATTTIPSTPMSIESIEDHQYHNSFLAMVERNQIPLSRNLLIILYLSHLLLQNNNTTITPYTSKFIHIQNCVGIDEFTGKSIYDIDINDTYFVIHSLVIVTFLRSFLMKWCFEPFASKFCHIHSKKAKTRFAEQSWSFVYYSISFIFGVVLYWDSPYYNNLDQVYINWPNHYMSWEFKTYYLVSMGFWLQQIFVLNVEKPRKDHYQMFSHHIITCLLIIGSYYYYYFRIGHLILMIMDSVDIFLAAAKMLKYAGFSNACDAMFLLFLVSWIVLRHGVYNYIFYHAWYKSVDLMKNGQCVEGLMQKRCWTPVVIDTFLGLLGGLQIITCIWMYLILKVAYKVVTGSGAEDVRSDEDDTDIELEEEEKEEEEEEEVGQPIFVEKKEEEVVLEIEEDKYSFERDSFSSSSESTLDEKKDIRKRKV